(19) United States
(12) Patent Application Publication
(10) Pub. No.: US 2006/0160175 A1
(43) Pub. Date: Jul. 20, 2006

(54) Compositions of orthogonal leucyl-trna and aminoacyl-trna synthetase pairs and uses thereof

(75) Inventors: J. Christopher Anderson, San Francisco, CA (US); Peter G. Schultz, La Jolla, CA (US)

Correspondence Address: QUINE INTELLECTUAL PROPERTY LAW GROUP, P.C., P O BOX 458, ALAMEDA, CA, 94501, US

(73) Assignee: The Scripps Research Institute

(21) Appl. No.: 10/563,655

(22) PCT Filed: Jul. 7, 2004
(86) PCT No.: PCT/US04/22061

§ 371 Date: Jan. 5, 2006

Related U.S. Application Data
(60) Provisional application No. 60/485,451, filed on Jul. 7, 2003.
(60) Provisional application No. 60/488,215, filed on Jul. 16, 2003.

(51) Int. Cl. C12P 21/06; C07H 21/04; C12P 19/34; C12N 9/22; C12N 15/74; C12N 1/21

(52) U.S. Cl. 435/69.1; 435/199; 435/252.33; 435/488; 536/23.2; 435/91.1

(57) ABSTRACT

Compositions and methods of producing components of protein biosynthetic machinery that include leucyl orthogonal tRNAs, leucyl orthogonal aminoacyl-tRNA synthetases, and orthogonal pairs of leucyl tRNAs/synthetases are provided. Methods for identifying these orthogonal pairs are also provided along with methods of producing proteins using these orthogonal pairs.

### CROSS-REFERENCE TO RELATED APPLICATIONS

This application claims priority to and benefit of Provisional Patent Application U.S. Ser. No. 60/485,451, filed Jul. 7, 2003; and to Provisional Patent Application U.S. Ser. No. 60/488,215, filed Jul. 16, 2003, the disclosures of which are incorporated herein by reference in their entirety for all purposes.

### STATEMENT AS TO RIGHTS TO INVENTIONS MADE UNDER FEDERALLY SPONSORED RESEARCH AND DEVELOPMENT

This invention was made with government support under Grant No. GM 62159 from the National Institutes of Health. The government may have certain rights to this invention.

### FIELD OF THE INVENTION

The invention pertains to the field of translation biochemistry. The invention relates to methods for producing and compositions of orthogonal leucyl tRNAs, orthogonal leucyl aminoacyl-tRNA synthetases and pairs thereof. The invention also relates to methods of producing proteins in cells using such pairs and related compositions.

### BACKGROUND OF THE INVENTION

The genetic code of every known organism, from bacteria to humans, encodes the same twenty common amino acids. Different combinations of the same twenty natural amino acids form proteins that carry out virtually all the complex processes of life, from photosynthesis to signal transduction and the immune response. In order to study and modify protein structure and function, scientists have attempted to manipulate both the genetic code and the amino acid sequence of protein. However, it has been difficult to remove the constraints imposed by the genetic code that limit proteins to twenty genetically encoded standard building blocks (with the rare exception of selenocysteine (see, e.g., A. Bock et al., (1991), Molecular Microbiology 5:515-20) and pyrrolysine (see, e.g., G. Srinivasan, et al., (2002), Science 296:1459-62).

Some progress has been made to remove these constraints, although this progress has been limited and the ability to rationally control protein structure and function is still in its infancy. For example, chemists have developed methods and strategies to synthesize and manipulate the structures of small molecules (see, e.g., E. J. Corey, & X.-M. Cheng, The Logic of Chemical Synthesis (Wiley-Interscience, New York, 1995)). Total synthesis (see, e.g., B. Merrifield, (1986), Science 232:341-7 (1986)), and semi-synthetic methodologies (see, e.g., D. Y. Jackson et al., (1994) Science 266:243-7; and, P. E. Dawson, & S. B. Kent, (2000), Annual Review of Biochemistry 69:923-60), have made it possible to synthesize peptides and small proteins, but these methodologies have limited utility with proteins over 10 kilo Daltons (kDa). Mutagenesis methods, though powerful, are restricted to a limited number of structural changes. In a number of cases, it has been possible to competitively incorporate close structural analogues of common amino acids throughout proteins. See, e.g., R. Furter, (1998), Protein Science 7:419-26; K. Kirshenbaum, et al., (2002), ChemBioChem 3:235-7; and, V. Doring et al., (2001), Science 292:501-4.

Early work demonstrated that the translational machinery of E. coli would accommodate amino acids similar in structure to the common twenty. See, Hortin, G., and Boime, I. (1983) Methods Enzymol. 96:777-784. This work was further extended by relaxing the specificity of endogenous E. coli synthetases so that they activate unnatural amino acids as well as their cognate natural amino acid. Moreover, it was shown that mutations in editing domains could also be used to extend the substrate scope of the endogenous synthetase. See, Doring, V., et al., (2001) Science 292:501-504. However, these strategies are limited to recoding the genetic code rather than expanding the genetic code and lead to varying degrees of substitution of one of the common twenty amino acids with an unnatural amino acid.

Later it was shown that unnatural amino acids could be site-specifically incorporated into proteins in vitro by the addition of chemically aminoacylated orthogonal amber suppressor tRNAs to an in vitro transcription/translation reaction. See, e.g., Noren, C. J., et al. (1989) Science 244:182-188; Bain, J. D., et al., (1989) J. Am. Chem. Soc. 111:8013-8014; Dougherty, D. A. (2000) Curr. Opin. Chem. Biol. 4, 645-652; Cornish, V. W., et al. (1995) Angew. Chem., Int. Ed. 34:621-633; J. A. Ellman, et al., (1992), Science 255:197-200; and, D. Mendel, et al., (1995), Annual Review of Biophysics and Biomolecular Structure 24:435-462. These studies show that the ribosome and translation factors are compatible with a large number of unnatural amino acids, even those with unusual structures. Unfortunately, the chemical aminoacylation of tRNAs is difficult, and the stoichiometric nature of this process severely limited the amount of protein that could be generated.

Unnatural amino acids have been microinjected into cells. For example, unnatural amino acids were introduced into the nicotinic acetylcholine receptor in Xenopus oocytes (e.g., M. W. Nowak, et al. (1998), In vivo incorporation of unnatural amino acids into ion channels in Xenopus oocyte expression system, Method Enzymol. 293:504-529) by microinjection of a chemically misacylated Tetrahymena thermophila tRNA (e.g., M. E. Saks, et al. (1996), An engineered Tetrahymena tRNAGln for in vivo incorporation of unnatural amino acids into proteins by nonsense suppression, J. Biol. Chem. 271:23169-23175), and the relevant mRNA. See, also, D. A. Dougherty (2000), Unnatural amino acids as probes of protein structure and function, Curr. Opin. Chem. Biol. 4:645-652. Unfortunately, this methodology is limited to proteins in cells that can be microinjected, and, because the relevant tRNA is chemically acylated in vitro, and cannot be re-acylated, the yields of protein are very low.

To overcome these limitations, new components, e.g., orthogonal tRNAs, orthogonal aminoacyl-tRNA synthetases and pairs thereof, were added to the protein biosynthetic machinery of the prokaryote Escherichia coli (E. coli) (see e.g., L. Wang, et al., (2001), Science 292:498-500), which allowed genetic encoding of unnatural amino acids in vivo. A number of new amino acids with novel chemical, physical or biological properties, including photoaffinity labels and photoisomerizable amino acids, photocrosslinking amino acids (see, e.g., Chin, J. W., et al. (2002) Proc. Natl. Acad. Sci. U.S. A. 99:11020-11024; and, Chin, J. W., et al., (2002) J. Am. Chem. Soc. 124:9026-9027), keto amino acids (see, e.g., Wang, L., et al., (2003) Proc. Natl. Acad. Sci. U.S.A. 100:56-61), heavy atom containing amino acids, and glycosylated amino acids have been incorporated efficiently and with high fidelity into proteins in E. coli in response to, e.g., the amber codon (TAG), using this methodology.

Several other orthogonal pairs have been reported. Glutaminyl (see, e.g., Liu, D. R., and Schultz, P. G. (1999) Proc. Natl. Acad. Sci. U.S.A. 96:4780-4785), aspartyl (see, e.g., Pastrnak, M., et al., (2000) Helv. Chim. Acta 83:2277-2286), and tyrosyl (see, e.g., Ohno, S., et al., (1998) J. Biochem. (Tokyo, Jpn.) 124:1065-1068; and, Kowal, A. K., et al., (2001) Proc. Natl. Acad. Sci. U.S.A. 98:2268-2273) systems derived from S. cerevisiae tRNAs and synthetases have been described for the potential incorporation of unnatural amino acids in E. coli. Systems derived from the E. coli glutaminyl (see, e.g., Kowal, A. K., et al., (2001) Proc. Natl. Acad. Sci. U.S.A. 98:2268-2273) and tyrosyl (see, e.g., Edwards, H., and Schimmel, P. (1990) Mol. Cell. Biol. 10:1633-1641) synthetase have been described for use in S. cerevisiae. The E. coli tyrosyl system has been used for the incorporation of 3-iodo-L-tyrosine in vivo, in mammalian cells. See, Sakamoto, K., et al., (2002) Nucleic Acids Res. 30:4692-4699. Typically, these systems have made use of the amber stop codon. To further expand the genetic code, there is a need to develop improved and/or additional components of the biosynthetic machinery, e.g., additional orthogonal tRNAs, orthogonal aminoacyl-tRNA synthetases, and/or unique codons. This invention fulfills these and other needs, as will be apparent upon review of the following disclosure.

### SUMMARY OF THE INVENTION

To expand the genetic code, the invention provides compositions of and methods for producing orthogonal leucyl-tRNAs, orthogonal leucyl aminoacyl-tRNA synthetases and pairs thereof. These translational components can be used to incorporate a selected amino acid in a specific position in a growing polypeptide chain (during nucleic acid translation) in response to a selector codon.

Compositions of the invention include a composition comprising an orthogonal leucyl-tRNA (leucyl-O-tRNA), where the leucyl O-tRNA comprises an anticodon loop comprising a CU(X)ₙ XXXAA sequence, and comprises at least about a 25% suppression activity in presence of a cognate synthetase in response to a selector codon as compared to a comparable control (e.g., in the absence of the selector codon). In one embodiment, the selector codon is an amber codon, and the leucyl O-tRNA comprises a stem region comprising matched base pairs and a conserved discriminator base at position 73. This position is indicated in FIG. 4, Panel A. In one aspect, the CU(X)ₙ XXXAA sequence comprises CUCUAAA sequence and n=0. In another aspect, the leucyl O-tRNA comprises a C:G base pair at position 3:70.

In one embodiment, the selector codon is a four-base codon and the leucyl O-tRNA comprises a first pair selected from U28:A42, G28:C42 and/or C28:G42, and a second pair selected from G:49:C65 or C49:G65, where the numbering corresponds to that indicated in FIG. 4, Panel A. In one aspect, the CU(X)ₙ XXXAA sequence comprises a CUUCCUAA sequence and n=1. In another aspect, the first pair is C28:G42 and the second pair is C49:G65. In one embodiment, the CU(X)ₙ XXXAA sequence comprises a CUUCAAA sequence and n=0, and the selector codon is an opal codon.

A composition comprising a leucyl O-tRNA can further include an orthogonal leucyl aminoacyl-tRNA synthetase (leucyl O-RS), where the leucyl O-RS preferentially aminoacylates the leucyl O-tRNA with a selected amino acid. In certain embodiments, a composition including a leucyl O-tRNA can further include a (e.g., in vitro or in vivo) translation system.

A composition of the invention also includes a cell (e.g., a non-eukaryotic cell (e.g., an E. coli cell), or a eukaryotic cell) comprising a translation system. The translation system includes an orthogonal leucyl-tRNA (leucyl-O-tRNA), where the leucyl-O-tRNA comprises at least about a 25% suppression activity in presence of a cognate synthetase in response to a selector codon as compared to a control lacking the selector codon; an orthogonal aminoacyl-leucyl-tRNA synthetase (leucyl-O-RS); and, a first selected amino acid. In these cells, the leucyl O-tRNA comprises an anticodon loop comprising a CU(X)ₙ XXXAA sequence and recognizes the first selector codon and the leucyl O-RS preferentially aminoacylates the leucyl O-tRNA with the first selected amino acid. In some embodiments, the cell translation system comprises a leucyl-O-tRNA and cognate synthetase, or a conservative variant thereof, where these components are at least 50% as effective at suppressing a selector codon as a leucyl O-tRNA of SEQ ID NO: 3, 6, 7 or 12, in combination with a cognate synthetase.

In certain embodiments, the cell can further include an additional different O-tRNA/O-RS pair and a second selected amino acid, where the O-tRNA recognizes a second selector codon and the O-RS preferentially aminoacylates the O-tRNA with the second selected amino acid. In one embodiment, the cell further comprises a nucleic acid that comprises a polynucleotide that encodes a polypeptide of interest, where the polynucleotide comprises/encodes a selector codon that is recognized by the leucyl O-tRNA.

In one embodiment, an E. coli cell includes an orthogonal leucyl-tRNA (leucyl-O-tRNA), where the leucyl-O-tRNA comprises at least about a 25% suppression activity in presence of a cognate synthetase in response to a selector codon as compared to a control lacking the selector codon; and an orthogonal leucyl aminoacyl-tRNA synthetase (leucyl-O-RS), where the O-RS preferentially aminoacylates the O-tRNA with a selected amino acid. The E. coli cell also includes the selected amino acid, and, a nucleic acid that comprises a polynucleotide that encodes a polypeptide of interest, where the polynucleotide comprises a selector codon that is recognized by the leucyl O-tRNA. In one example, the leucyl O-tRNA is derived from Halobacterium sp NRC-1 and the leucyl O-RS is derived from Methanobacterium thermoaautotropicum.

In certain embodiments of the invention, a leucyl O-tRNA of the invention comprises or is encoded by a polynucleotide sequence as set forth in any one of SEQ ID NO.: 3, 6, 7 or 12, or a complementary polynucleotide sequence thereof. In some embodiments, the leucyl-O-tRNA and cognate synthetase, or a conservative variant thereof, are at least 50% as effective at suppressing a selector codon as a leucyl O-tRNA of SEQ ID NO: 3, 6, 7 or 12, in combination with a cognate synthetase. In the case of tRNA molecules, thymine (t) is, of course, replaced by uracil (u). In certain embodiments, a leucyl O-RS comprises an amino acid sequence as set forth in any one of SEQ ID NO.: 15 or 16, or a conservative variation thereof. In one embodiment, the leucyl O-RS or a portion thereof is encoded by a polynucleotide sequence as set forth in any one of SEQ ID NO.: 13 or 14, a conservative variant of SEQ ID NO: 13 or 14, or a complementary polynucleotide sequence thereof.

The leucyl O-tRNA and/or the leucyl O-RS of the invention can be derived from any of a variety of organisms (e.g., both eukaryotic and non-eukaryotic organisms). For example, the leucyl O-tRNA is derived from an archael tRNA (e.g., from Halobacterium sp NRC-1) and/or the leucyl O-RS is derived from a non-eukaryotic organism (e.g., Methanobacterium thermoaautotropicum).

Polynucleotides are also a feature of the invention. A polynucleotide of the invention includes a polynucleotide comprising a nucleotide sequence as set forth in any one of SEQ ID NO.: 1-2, 4-7, 12, and/or is complementary to or that encodes a polynucleotide sequence of the above. A polynucleotide of the invention also includes a nucleic acid that hybridizes to a polynucleotide described above, under highly stringent conditions over substantially the entire length of the nucleic acid. A polynucleotide of the invention also includes a polynucleotide that is, e.g., at least 75%, at least 80%, at least 90%, at least 95%, at least 98% or more identical to that of a naturally occurring leucyl tRNA or a consensus sequence of multiple naturally occurring leucyl tRNAs, e.g., the leucyl tRNA of SEQ ID NO: 12, and comprises an anticodon loop comprising a CU(X)ₙ XXXAA sequence, an stem region lacking noncanonical base pairs and a conserved discriminator base at position 73. A polynucleotide of the invention also includes a polynucleotide that is, e.g., at least 75%, at least 80%, at least 90%, at least 95%, at least 98% or more identical to that of a naturally occurring leucyl tRNA and comprises an anticodon loop comprising a CUUCCUAA sequence, a first pair selected from T28:A42, G28:C42 and/or C28:G42, and a second pair selected from G:49:C65 or C49:G65, where the numbering corresponds to that indicated in FIG. 4, Panel A. Polynucleotides that are, e.g., at least 80%, at least 90%, at least 95%, at least 98% or more identical to any of the above and/or a polynucleotide comprising a conservative variation of any the above or in Table 3 are also polynucleotides of the invention.

Vectors comprising or encoding a polynucleotide of the invention are also a feature of the invention. For example, a vector optionally includes any of: a plasmid, a cosmid, a phage, a virus, an expression vector, and/or the like. A cell comprising a vector of the invention is also a feature of the invention.

Methods of producing an orthogonal tRNA (O-tRNA), e.g., a leucyl O-tRNA, are also a feature of the invention. An O-tRNA, e.g., a leucyl O-tRNA, produced by the method is also a feature of the invention. For example, a method includes mutating an anticodon loop on members of a pool of tRNAs (e.g., pool of leucyl tRNAs) to allow recognition of a selector codon, thereby providing a plurality of potential O-tRNAs; and analyzing secondary structure of at least one member of the plurality potential O-tRNA to identify non-canonical base pairs in the secondary structure, and, optionally, mutating the non-canonical base pairs (e.g., mutating the non-canonical base pairs to canonical base pairs). In one embodiment, the non-canonical base pairs are located in stem region of the secondary structure. A population of cells of a first species, where the cells individually comprise at least one member of the plurality of potential O-tRNAs are subjected to a negative selection, thereby eliminating cells that comprise a member of the plurality of potential O-tRNAs that is aminoacylated by an aminoacyl-tRNA synthetase (RS) that is endogenous to the cell, and providing a pool of tRNAs that are orthogonal to the cell of the first species. In certain embodiments, the selector codon includes an amber codon, an opal codon, a four base codon, etc. The method can further include adding an additional sequence (CCA) to a 3′ terminus of each of the pool of tRNAs and/or measuring suppression activity.

In one embodiment, the pool of tRNAs is obtained by aligning a plurality of tRNA sequences; determining a consensus sequence; and generating a library of mutant tRNAs using the consensus sequence, where the pool of tRNAs comprise the library of mutant tRNAs.

In certain embodiments, the subjecting step comprises a polynucleotide that encodes a negative selection marker. In one embodiment, the polynucleotide that encodes the negative selection marker comprises at least one selector codon. For example, a negative selection marker includes, but is not limited to, β-lactamase, β-galactosidase, and/or the like. In certain embodiments, the negative selection marker fluoresces or catalyzes a luminescent reaction in the presence of a suitable reactant. In another embodiment, a product of the negative selection marker is detected by fluorescence-activated cell sorting (FACS) or by luminescence. Optionally, the negative selection marker includes an affinity based screening marker. In certain embodiments, the subjecting step comprises growing the population of cells in the presence of a selective agent (e.g., an antibiotic, such as ampicillin).

In certain embodiments, the method further comprises subjecting to positive selection a second population of cells of the first species. The cells comprise a member of the pool of tRNAs that are orthogonal to the cell of the first species, a cognate aminoacyl-tRNA synthetase, and a positive selection marker. Cells are selected/screened for cells that comprise a member of the pool of tRNAs that is aminoacylated by the cognate aminoacyl-tRNA synthetase and that shows a desired response in the presence of the positive selection marker, thereby providing an O-tRNA.

Methods for identifying an orthogonal aminoacyl-tRNA synthetase (O-RS), e.g., a leucyl O-RS, for use with an O-tRNA, e.g., a leucyl O-tRNA, are also a feature of the invention. For example, a method includes subjecting to positive selection a population of cells of a first species, where the cells each comprise: 1) a member of a plurality of aminoacyl-tRNA synthetases (RSs), where the plurality of RSs comprise mutant RSs, RSs derived from a species other than the first species or both mutant RSs and RSs derived from a species other than the first species; 2) the orthogonal tRNA (O-tRNA) (e.g., from a species other than the first species, from at least a second species, etc.); and 3) a polynucleotide that encodes a positive selection marker and comprises at least one selector codon. In one embodiment, the plurality of RSs comprises leucyl RSs. In certain embodiments, the O-tRNA comprises a leucyl O-tRNA (e.g., where leucyl O-tRNA includes at least about a 25% suppression activity in presence of a cognate synthetase in response to a selector codon as compared to a control lacking the cognate synthetase).

Cells are selected or screened for those that show an enhancement in suppression efficiency compared to cells lacking or having a reduced amount of the member of the plurality of RSs. These selected/screened cells comprise an active RS that aminoacylates the O-tRNA. The level of aminoacylation (in vitro or in vivo) by the active RS of a first set of tRNAs from the first species is compared to the level of aminoacylation (in vitro or in vivo) by the active RS of a second set of tRNAs from a second species; where the level of aminoacylation is determined by a detectable substance (e.g., a labeled amino acid). The active RS that more efficiently aminoacylates the second set of tRNAs compared to the first set of tRNAs is selected, thereby providing the orthogonal aminoacyl-tRNA synthetase, e.g., leucyl O-RS, for use with the O-tRNA, e.g., the leucyl O-tRNA. An orthogonal aminoacyl-tRNA synthetase identified by the method is also a feature of the invention.

Methods of producing a protein in a cell with a selected amino acid at a specified position are also a feature of the invention. For example, a method includes growing, in an appropriate medium, a cell, where the cell comprises a nucleic acid that comprises at least one selector codon and encodes a protein; and, providing the selected amino acid. The cell further comprises: an orthogonal leucyl-tRNA (leucyl-O-tRNA) that functions in the cell and recognizes the selector codon; and, an orthogonal leucyl aminoacyl-tRNA synthetase (leucyl O-RS) that preferentially aminoacylates the leucyl-O-tRNA with the selected amino acid. Typically, the leucyl-O-tRNA comprises at least about a 25% suppression activity in presence of a cognate synthetase in response to a selector codon as compared to a control lacking the cognate synthetase. A protein produced by this method is also a feature of the invention.

### DEFINITIONS

Before describing the invention in detail, it is to be understood that this invention is not limited to particular biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells; reference to “bacteria” includes mixtures of bacteria, and the like.

Unless defined herein and below in the reminder of the specification, all technical and scientific terms used herein have the same meaning as commonly understood by one of ordinary skill in the art to which the invention pertains.

Orthogonal leucyl-tRNA: As used herein, an orthogonal leucyl-tRNA (leucyl-O-tRNA) is a tRNA that is orthogonal to a translation system of interest, where the tRNA is: (1) identical or substantially similar to a naturally occurring leucyl tRNA, (2) derived from a naturally occurring leucyl tRNA by natural or artificial mutagenesis (3) derived by any process that takes a sequence of a wild-type or mutant leucyl tRNA sequence of (1) or (2) into account, (4) homologous to a wild-type or mutant leucyl tRNA; (5) homologous to any example tRNA that is designated as a substrate for a leucyl tRNA synthetase in Table 3, or (6) a conservative variant of any example tRNA that is designated as a substrate for a leucyl tRNA synthetase in Table 3. The leucyl tRNA can exist charged with an amino acid, or in an uncharged state. It is also to be understood that a “leucyl-O-tRNA” optionally is charged (aminoacylated) by a cognate synthetase with an amino acid other than leucine. Indeed, it will be appreciated that a leucyl-O-tRNA of the invention is advantageously used to insert essentially any amino acid, whether natural or artificial, into a growing polypeptide, during translation, in response to a selector codon.

Orthogonal leucyl amino acid synthetase: As used herein, an orthogonal leucyl amino acid synthetase (leucyl O-RS) is an enzyme that preferentially aminoacylates the leucyl-O-tRNA with an amino acid in a translation system of interest. The amino acid that the leucyl O-RS loads onto the leucyl O-tRNA can be any amino acid, whether natural or artificial, and is not limited herein. The synthetase is optionally the same as or homologous to a naturally occurring leucyl amino acid synthetase, or the same as or homologous to a synthetase designated as a leucyl O-RS in Table 3. For example, the leucyl O-RS can be a conservative variant of a leucyl O-RS of Table 3, and/or can be at least 50%, 60%, 70%, 80%, 90%, 95%, 98%, 99% or more identical in sequence to a leucyl O-RS of Table 3.

Homologous: Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. For example, any naturally occurring nucleic acid can be modified by any available mutagenesis method to include one or more selector codon. When expressed, this mutagenized nucleic acid encodes a polypeptide comprising one or more selected amino acid, e.g. unnatural amino acid. The mutation process can, of course, additionally alter one or more standard codon, thereby changing one or more standard amino acid in the resulting mutant protein as well. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95%, or 99% or more, can also be used to establish homology. Methods for determining sequence similarity percentages (e.g., BLASTP and BLASTN using default parameters) are described herein and are generally available.

Orthogonal: As used herein, the term “orthogonal” refers to a molecule (e.g., an orthogonal tRNA (O-tRNA) and/or an orthogonal aminoacyl tRNA synthetase (O-RS)) that functions with endogenous components of a cell with reduced efficiency as compared to a corresponding molecule that is endogenous to the cell or translation system, or that fails to function with endogenous components of the cell. In the context of tRNAs and aminoacyl-tRNA synthetases, orthogonal refers to an inability or reduced efficiency, e.g., less than 20% efficiency, less than 10% efficiency, less than 5% efficiency, or less than 1% efficiency, of an orthogonal tRNA to function with an endogenous tRNA synthetase compared to the ability of an endogenous tRNA to function with the endogenous tRNA synthetase; or of an orthogonal aminoacyl-tRNA synthetase to function with an endogenous tRNA compared to the ability of an endogenous tRNA synthetase to function with the endogenous tRNA. The orthogonal molecule lacks a functionally normal endogenous complementary molecule in the cell. For example, an orthogonal tRNA in a cell is aminoacylated by any endogenous RS of the cell with reduced or even undetectable efficiency, when compared to aminoacylation of an endogenous tRNA by the endogenous RS. In another example, an orthogonal RS aminoacylates any endogenous tRNA in a cell of interest with reduced or even undetectable efficiency, as compared to aminoacylation of the endogenous tRNA by an endogenous RS. A second orthogonal molecule can be introduced into the cell that functions with the first orthogonal molecule. For example, an orthogonal tRNA/RS pair includes introduced complementary components that function together in the cell with an efficiency (e.g., 45% efficiency, 50% efficiency, 60% efficiency, 70% efficiency, 75% efficiency, 80% efficiency, 90% efficiency, 95% efficiency, or 99% or more efficiency) as compared to that of a control, e.g., a corresponding tRNA/RS endogenous pair, or an active orthogonal pair (e.g., a tyrosyl orthogonal tRNA/RS pair).

Cognate: The term “cognate” refers to components that function together, e.g., a leucyl tRNA and a leucyl aminoacyl-tRNA synthetase. The components can also be referred to as being complementary.

Preferentially aminoacylates: The term “preferentially aminoacylates” refers to an efficiency, e.g., 70% efficient, 75% efficient, 85% efficient, 90% efficient, 95% efficient, or 99% or more efficient, at which an O-RS aminoacylates an O-tRNA with a selected amino acid, e.g., an unnatural amino acid, as compared to the O-RS aminoacylating a naturally occurring tRNA or a starting material used to generate the O-tRNA.

Selector codon: The term “selector codon” refers to codons recognized by the O-tRNA in the translation process and not recognized by an endogenous tRNA. The O-tRNA anticodon loop recognizes the selector codon on the mRNA and incorporates its amino acid, e.g., a selected amino acid, such as an unnatural amino acid, at this site in the polypeptide. Selector codons can include, e.g., nonsense codons, such as, stop codons, e.g., amber, ochre, and opal codons; four or more base codons; rare codons; codons derived from natural or unnatural base pairs and/or the like.

Suppressor tRNA: A suppressor tRNA is a tRNA that alters the reading of a messenger RNA (mRNA) in a given translation system, e.g., by providing a mechanism for incorporating an amino acid into a polypeptide chain in response to a selector codon. For example, a suppressor tRNA can read through, e.g., a stop codon, a four base codon, or a rare codon.

Suppression activity: As used herein, the term “suppression activity” refers, in general, to the ability of a tRNA (e.g., a suppressor tRNA) to allow translational read-through of a codon (e.g. a selector codon that is an amber codon or a 4-or-more base codon) that would otherwise result in the termination of translation or mistranslation (e.g., frame-shifting). Suppression activity of a suppressor tRNA can be expressed as a percentage of translational read-through observed compared to a second suppressor tRNA, or as compared to a control system, e.g., a control system lacking an O-RS.

The present invention provides various means by which suppression activity can be quantitated. Percent suppression of a particular OtRNA and ORS against a selector codon (e.g., an amber codon) of interest refers to the percentage of activity of a given expressed test marker (e.g., LacZ), that includes a selector codon, in a nucleic acid encoding the expressed test marker, in a translation system of interest, where the translation system of interest includes an O-RS and an O-tRNA, as compared to a positive control construct, where the positive control lacks the O-tRNA, the O-RS and the selector codon. Thus, for example, if an active positive control marker construct that lacks a selector codon has an observed activity of X in a given translation system, in units relevant to the marker assay at issue, then percent suppression of a test construct comprising the selector codon is the percentage of X that the test marker construct displays under essentially the same environmental conditions as the positive control marker was expressed under, except that the test marker construct is expressed in a translation system that also includes the O-tRNA and the O-RS. Typically, the translation system expressing the test marker also includes an amino acid that is recognized by the O-RS and O-tRNA. Optionally, the percent suppression measurement can be refined by comparison of the test marker to a “background” or “negative” control marker construct, which includes the same selector codon as the test marker, but in a system that does not include the O-tRNA, O-RS and/or relevant amino acid recognized by the O-tRNA and/or O-RS. This negative control is useful in normalizing percent suppression measurements to account for background signal effects from the marker in the translation system of interest.

Suppression efficiency can be determined by any of a number of assays known in the art. For example, a β-galactosidase reporter assay can be used, e.g., a derivatized lacZ plasmid (where the construct has a selector codon in the lacZ nucleic acid sequence) is introduced into cells from an appropriate organism (e.g., an organism where the orthogonal components can be used) along with plasmid comprising an O-tRNA of the invention. A cognate synthetase can also be introduced (either as a polypeptide or a polynucleotide that encodes the cognate synthetase when expressed). The cells are grown in media to a desired density, e.g., to an OD₆₀₀ of about 0.5, and β-galactosidase assays are performed, e.g., using the BetaFluor™ β-Galactosidase Assay Kit (Novagen). Percent suppression can be calculated as the percentage of activity for a sample relative to a comparable control, e.g., the value observed from the derivatived lacZ construct, where the construct has a corresponding sense codon at desired position rather than a selector codon.

Translation system: The term “translation system” refers to the components that incorporate an amino acid into a growing polypeptide chain (protein). Components of a translation system can include, e.g., ribosomes, tRNAs, synthetases, mRNA and the like. The O-tRNA and/or O-RS of the invention can be added to or be a part of an in vitro or in vivo translation system, e.g., in a non-eukaryotic cell, e.g., a bacterium (such as E. coli), or in a eukaryotic cell, e.g., a yeast cell, a mammalian cell, a plant cell, an algae cell, a fungus cell, an insect cell, and/or the like.

Selected amino acid: The term “selected amino acid” refers to any desired naturally occurring amino acid or unnatural amino acid. As used herein, the term “unnatural amino acid” refers to any amino acid, modified amino acid, and/or amino acid analogue that is not one of the 20 common naturally occurring amino acids or seleno cysteine or pyrolysine.

Derived from: As used herein, the term “derived from” refers to a component that is isolated from or made using a specified molecule or organism, or information from the specified molecule or organism.

Positive selection or screening marker: As used herein, the term “positive selection or screening marker” refers to a marker that, when present, e.g., expressed, activated, or the like, results in identification of a cell with the positive selection marker from those without the positive selection marker.

Negative selection or screening marker: As used herein, the term “negative selection or screening marker” refers to a marker that, when present, e.g., expressed, activated or the like, allows identification of a cell that does not possess a specified property (e.g., as compared to a cell that does possess the property).

Reporter: As used herein, the term “reporter” refers to a component that can be used to identify and/or select target components of a system of interest. For example, a reporter can include a protein, e.g., an enzyme, that confers antibiotic resistance or sensitivity (e.g., β-lactamase, chloramphenicol acetyltransferase (CAT), and the like), a fluorescent screening marker (e.g., green fluorescent protein (e.g., (GFP), YFP, EGFP, RFP), a luminescent marker (e.g., a firefly luciferase protein), an affinity based screening marker, or positive or negative selectable marker genes such as lacZ, β-gal/lacZ (β-galactosidase), Adh (alcohol dehydrogenase), his3, ura3, leu2, lys2, or the like.

Eukaryote: As used herein, the term “eukaryote” refers to organisms belonging to the phylogenetic domain Eucarya, such as animals (e.g., mammals, insects, reptiles, birds, etc.), ciliates, plants (e.g., monocots, dicots, algae, etc.), fungi, yeasts, flagellates, microsporidia, protists, etc.

Non-eukaryote: As used herein, the term “non-eukaryote” refers to non-eukaryotic organisms. For example, a non-eukaryotic organism can belong to the Eubacteria (e.g., Escherichia coli, Thermus thermophilus, Bacillus stearothermophilus, etc.) phylogenetic domain, or the Archaea (e.g., Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domains.

Conservative variant: The term “conservative variant” in reference to a translation component such as an O-tRNA or O-RS refers to a translation component that has a substantailly similar activity as the component on which the conservative variant is similar to, e.g., an O-tRNA or O-RS, but has variations in the sequence as compared to the base component. For example, an O-RS will aminoacylate a complementary O-tRNA or a conservative variant O-tRNA with a selected amino acid, e.g., an unnatural amino acid, although the O-tRNA and the conservative variant O-tRNA do not have the same sequence. The conservative variant can have, e.g., one variation, two variations, three variations, four variations, or five or more variations in its sequence, as long as the conservative variant functionally interacts with a corresponding O-tRNA or O-RS in substantailly the same manner as the non-variant form.

Selection or screening agent: As used herein, the term “selection or screening agent” refers to an agent that, when present, allows for selection/screening of certain components from a population. For example, a selection or screening agent can be, but is not limited to, e.g., a nutrient, an antibiotic, a wavelength of light, an antibody, an expressed polynucleotide, or the like. The selection agent can be varied, e.g., by concentration, intensity, etc.

Encode: As used herein, the term “encode” refers to any process whereby the information in a polymeric macromolecule or sequence string is used to direct the production of a second molecule or sequence string that is different from the first molecule or sequence string. As used herein, the term is used broadly, and can have a variety of applications. In one aspect, the term “encode” describes the process of semi-conservative DNA replication, where one strand of a double-stranded DNA molecule is used as a template to encode a newly synthesized complementary sister strand by a DNA-dependent DNA polymerase.

In another aspect, the term “encode” refers to any process whereby the information in one molecule is used to direct the production of a second molecule that has a different chemical nature from the first molecule. For example, a DNA molecule can encode an RNA molecule (e.g., by the process of transcription incorporating a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

### BRIEF DESCRIPTION OF THE FIGURES

FIG. 1, Panels A, B and C schematically illustrate leucyl tRNAs and synthetases, and their phylogenetic relationships. Panel A provides a ClustalW analysis of aminoacyl-tRNA synthetases, where Archaeal tRNA synthetases are labeled using a dashed line, prokaryotic using a solid line, and eukaryotic sequences using a dotted line. This analysis reveals the halobacterial synthetase to be unusual in its homology to prokaryotic rather than archaeal and eukaryotic synthetases. Panel B provides a ClustalW analysis of Halobacterial tRNAs which all share high homology to other archaeal tRNAs. Dendrograms were generated using the program PhyloDraw. Panel C provides a sequence alignment of multiple sequences of the family of archaeal leucyl tRNAs examined as potential orthogonal suppressors. Sequences examined as potential amber suppressors by changing the anticodon (boxed) to CUA are shown in bold as is the consensus sequence. The highly conserved positions G37 and A73 are indicated with underlining.

FIG. 2 provides a histogram showing the identification of a leucyl orthogonal pair. The suppression efficiency of seven synthetases expressed with 5 orthogonal amber suppressor reporter constructs was measured using a β-lactamase amber suppression assay.

FIG. 3, Panels A and B provide graphs illustrating aminoacylation in vitro by archaeal leucyl-tRNA synthetases. Panel A illustrates charging of crude total halobacterial tRNA determined by aminoacylation assays with [³H] leucine by AfLRS (▪), MjLRS (●), MtLRS (▴), EcLRS (♦), and no synthetase (□). Panel B illustrates charging of crude total E. coli tRNA.

FIG. 4, Panels A and B illustrates the optimization of suppressor tRNAs. Panel A illustrates regions (shown in boxes) of the halobacterial orthogonal tRNA subjected to mutagenesis in an effort to improve the efficiency or selectivity of TAG and AGGA suppressor tRNAs. Panel B illustrates that active mutant TAG suppressors identified by positive selection conserve A73. Less cross-reactive mutants identified by a double-sieve selection strategy conserve a C3:G70 base pair. The most active and selective suppressor tRNA is shown with double boxes.

FIG. 5 illustrates a consensus-derived frameshift suppressor. A consensus sequence was obtained by multiple sequence alignment of all known archaeal leucyl tRNAs, and the anticodon loop is changed to UCUCCUAA. The variations observed for tRNAs identified by selection are shown in boxes. The most active mutations are shown with double boxes.

### DETAILED DESCRIPTION

In order to add additional unnatural amino acids to the genetic code in vivo, “orthogonal pairs” of an aminoacyl-tRNA synthetase and a tRNA are needed that can function efficiently in the translational machinery. Desired characteristics of the orthogonal pairs include tRNA that decode or recognize only a specific new codon, e.g., a selector codon, that is not decoded by any endogenous tRNA, and aminoacyl-tRNA synthetases that preferentially aminoacylate (or charge) its cognate tRNA with only a specific selected amino acid, e.g., an unnatural amino acid. The O-tRNA is also not typically aminoacylated by endogenous synthetases. For example, in E. coli, an orthogonal pair will include an aminoacyl-tRNA synthetase that does not significantly cross-react with any of the endogenous tRNA, which there are 40 in E. coli, and an orthogonal tRNA that is not significantly aminoacylated by any of the endogenous synthetases, e.g., of which there are 21 in E. coli.

The O-tRNA is capable of mediating incorporation of a selected amino acid into a protein that is encoded by a polynucleotide, which comprises a selector codon that is recognized by the O-tRNA, e.g., in vivo. The anticodon loop of the O-tRNA recognizes the selector codon on an mRNA and incorporates its amino acid, e.g., a selected amino acid, such as an unnatural amino acid, at this site in the polypeptide. Any of a number of selector codons can be used with the invention. For example, selector codons can include, e.g., nonsense codons, such as, stop codons, e.g., amber, ochre, and opal codons; four or more base codons; rare codons; codons derived from natural or unnatural base pairs and/or the like. See also the section herein entitled “Selector codon.”

By using different selector codons, multiple orthogonal tRNA/synthetase pairs can be developed that allow the simultaneous incorporation of multiple selected amino acids, e.g., unnatural amino acids, using these different selector codons. This invention provides compositions of and methods for identifying and producing additional orthogonal tRNA-aminoacyl-tRNA synthetase pairs, e.g., leucyl O-tRNA/leucyl O-RSs, using any of a number of selector codons, e.g., an amber codon, an opal codon, an extended codon (such as a four-base codon), and the like.

Orthogonal Leucyl tRNA/Orthogonal Leucyl Aminoacyl-tRNA Synthetases and Pairs Thereof

Such translation systems of the invention generally comprise cells that include an orthogonal leucyl tRNA (leucyl O-tRNA), an orthogonal leucyl aminoacyl tRNA synthetase (leucyl O-RS), and a selected amino acid, e.g., an unnatural amino acid, where the leucyl O-RS aminoacylates the leucyl O-tRNA with the selected amino acid. An orthogonal pair of the invention is composed of a leucyl O-tRNA, e.g., a suppressor tRNA, a frameshift tRNA, or the like, and an leucyl O-RS. The leucyl-O-tRNA recognize a first selector codon and has at least about a 25% suppression activity in presence of a cognate synthetase in response to a selector codon as compared to a control lacking the cognate synthetase. The leucyl O-tRNA also comprises an anticodon loop comprising a CU(X) n XXXAA sequence. The cell uses the components to incorporate the selected amino acid into a growing polypeptide chain. For example, a nucleic acid that comprises a polynucleotide that encodes a polypeptide of interest can also be present, where the polynucleotide comprises a selector codon that is recognized by the leucyl O-tRNA. The translation system can also be an in vitro system.

Translation systems that are suitable for making proteins that include one or more selected amino acids, e.g., an unnatural amino acid, are described in International patent applications WO 2002/086075, entitled “METHODS AND COMPOSITION FOR THE PRODUCTION OF ORTHOGANOL tRNA-AMINOACYLtRNA SYNTHETASE PAIRS” and WO 2002/085923, entitled “IN VIVO INCORPORATION OF UNNATURAL AMINO ACIDS.” In addition, see International Application Number PCT/US2004/011786, filed Apr. 16, 2004. Each of these applications is incorporated herein by reference in its entirety. These translation systems can be adapted to the present invention by substituting the leucyl-O-RS and leucyl-O-tRNA provided herein.

In certain embodiments, a cell of teh invention, e.g., an E. coli cell, includes such a translation system of the invention. For example, an E. coli cell of the invention can include an orthogonal leucyl-tRNA (leucyl-O-tRNA), where the leucyl-O-tRNA comprises at least about a 25% suppression activity in presence of a cognate synthetase in response to a selector codon as compared to a control lacking the cognate synthetase; an orthogonal leucyl aminoacyl-tRNA synthetase (leucyl-O-RS); a selected amino acid; and, a nucleic acid that comprises a polynucleotide that encodes a polypeptide of interest, where the polynucleotide comprises a selector codon that is recognized by the leucyl O-tRNA.

The invention also features multiple O-tRNA/O-RS pairs in a cell, which allows incorporation of more than one selected amino acid. In certain embodiments, the cell can further include an additional different O-tRNA/O-RS pair and a second selected amino acid, where the O-tRNA recognizes a second selector codon and the O-RS preferentially aminoacylates the O-tRNA with the second selected amino acid. For example, a cell can further comprise, e.g., an amber suppressor tRNA-aminoacyl tRNA synthetase pair derived from the tyrosyl-tRNA synthetase of Methanococcus jannaschii.

The leucyl O-tRNA and/or the leucyl O-RS can be naturally occurring or can be derived by mutation of a naturally occurring tRNA and/or RS, e.g., which generates libraries of tRNAs and/or libraries of RSs, from a variety of organisms. For example, one strategy of producing an orthogonal leucyl tRNA/leucyl aminoacyl-tRNA synthetase pair involves importing a heterologous tRNA/synthetase pair from, e.g., a source other than the host cell, or multiple sources, into the host cell. The properties of the heterologous synthetase candidate include, e.g., that it does not charge any host cell tRNA, and the properties of the heterologous tRNA candidate include, e.g., that it is not aminoacylated by any host cell synthetase. In addition, the heterologous tRNA is orthogonal to all host cell synthetases.

A second strategy for generating an orthogonal pair involves generating mutant libraries from which to screen and/or select a leucyl O-tRNA or leucyl O-RS. These strategies can also be combined.

In various embodiments, the leucyl O-tRNA and leucyl O-RS are derived from at least one organism. In another embodiment, the leucyl O-tRNA is derived from a naturally occurring or mutated naturally occurring tRNA from a first organism and the leucyl O-RS is derived from naturally occurring or mutated naturally occurring RS from a second organism. In one embodiment, the first and second organism is different. For example, an orthogonal pair of the invention includes a leucyl-tRNA synthetase derived from Methanobacterium thermoautotrophicum, and a leucyl tRNA derived from an archael tRNA (e.g., from Halobacterium sp. NRC-1). Alternatively, the first and second organism are the same. See the section entitled “Sources and Hosts” herein for additional information.

In certain embodiments of the invention, a leucyl O-tRNA of the invention comprises or is encoded or transcribed by or from a polynucleotide sequence as set forth in any one of SEQ ID NO.: 3, 6, 7 or 12, or a complementary polynucleotide sequence thereof. In certain embodiments, a leucyl O-RS comprises an amino acid sequence as set forth in any one of SEQ ID NO.: 15 or 16, or a conservative variation thereof. The leucyl O-RS, or a portion thereof, can also be encoded or transcribed by or from a polynucleotide sequence as set forth in any one of SEQ ID NO.: 13 or 14, or a complementary polynucleotide sequence thereof. See also, the section entitled “Nucleic Acid and Polypeptide Sequence and Variants,” herein.

Orthogonal tRNA (O-tRNA)

An orthogonal leucyl tRNA (leucyl O-tRNA) mediates incorporation of a selected amino acid into a protein that is encoded by a polynucleotide that comprises a selector codon that is recognized by the leucyl O-tRNA, e.g., in vivo. A leucyl O-tRNA of the invention comprises an anticodon loop comprising a CU(X)ₙ XXXAA sequence.

The CU(X)ₙ XXXAA sequence is found in the anticodon loop, where X refers to any nucleotide, and (X)ₙ is optionally present. The n refers to a number of bases the anticodon loop is extended, based on the desired selector codon, e.g., a stop codon (n=0), an extended codon, such as a four- (n=1), five- (n=2), six- (n=3) base pair, etc.

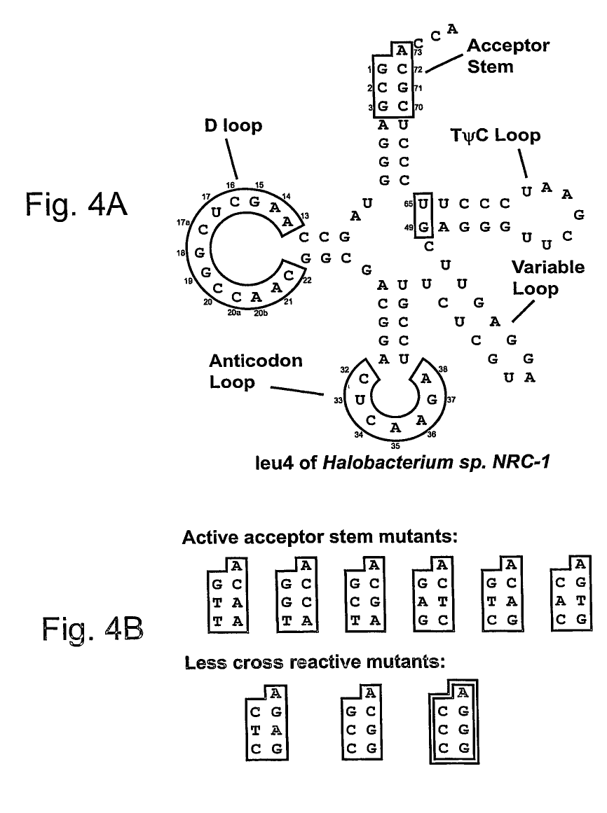

In one aspect of the invention, the CU(X)ₙ XXXAA sequence comprises CUCUAAA sequence (n=0), typically when the selector codon is an amber codon. In addition, the leucyl O-tRNA can include a stem region comprising matched base pairs and a conserved discriminator base (position 73). See, e.g., FIG. 4, Panel B. This position is indicated in e.g., FIG. 4, Panel A. The leucyl O-tRNA also optionally includes a C:G base pair at position 3:70.

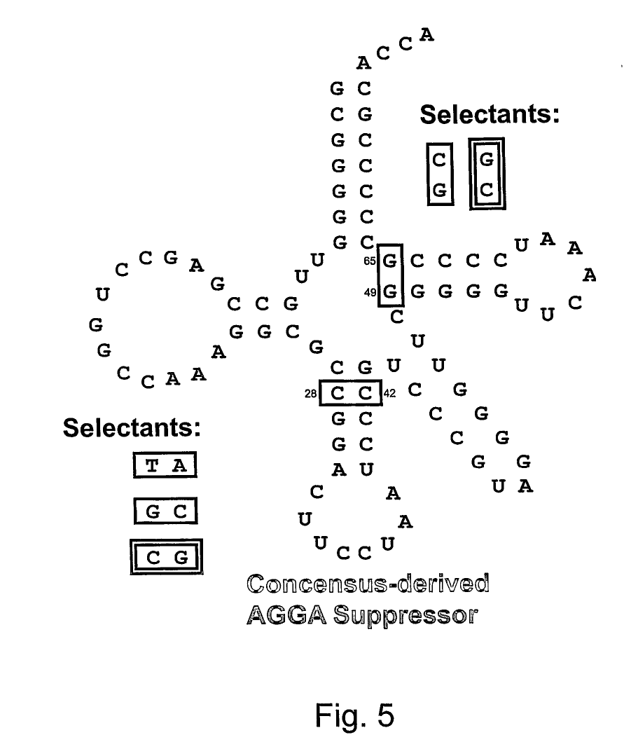

In one example, the CU(X)ₙ XXXAA sequence comprises a CUUCCUAA sequence, typically when the selector codon is a four-base codon. See, e.g., FIG. 5. The leucyl O-tRNA can also include a first pair selected from T28:A42, G28:C42 and/or C28:G42, and a second pair selected from G:49:C65 or C49:G65, where the numbering corresponds to that indicated in FIG. 4, Panel A. In one embodiment, C28:G42 is the first pair and C49:G65 is the second pair. When the selector codon is an opal codon, the CU(X)ₙ XXXAA sequence can comprises a CUUCAAA sequence.

A leucyl O-tRNA of the invention comprises at least about a 25% suppression activity in presence of a cognate synthetase in response to a selector codon, as compared to a control lacking the cognate synthetase. Suppression activity can be determined by any of a number of assays known in the art. For example, a β-galactosidase reporter assay can be used A derivative of a plasmid that expresses lacZ gene under the control of promoter is used, e.g., where the Leu-25 of the peptide VVLQRRDWEN of lacZ is replaced by a selector codon, e.g., TAG, TGA, AGGA, etc. codons, or sense codons (as a control) for tyrosine, serine, leucine, etc. The derivatived lacZ plasmid is introduced into cells from an appropriate organism (e.g., an organism where the orthogonal components can be used) along with plasmid comprising a O-tRNA of the invention. A cognate synthetase can also be introduced (either as a polypeptide or a polynucleotide that encodes the cognate synthetase when expressed). The cells are grown in media to a desired density, e.g., to an OD₆₀₀ of about 0.5, and β-galactosidase assays are performed, e.g., using the BetaFluor™ β-Galactosidase Assay Kit (Novagen). Percent suppression is calculated as the percentage of activity for a sample relative to a comparable control, e.g., the value observed from the derivatived lacZ construct, where the construct has a corresponding sense codon at desired position rather than a selector codon.

Examples of leucyl O-tRNAs of the invention are transcribed from any one of SEQ ID NO.: 1-7 and/or 12. See, Table 3 and Example 2, herein, for sequences of exemplary O-RS and O-tRNA molecules. In the tRNA molecule, Thymine (T) is replace with Uracil (U); the tRNAs have the same sequence, except for the usual substitution of U's for T's. One of skill will appreciate that the RNA and DNA versions of a tRNA are often referred to simply by reference to the DNA sequence that corersponds to the RNA form of the tRNA. Any time a DNA form of a tRNA is given, one of skill will easily be able to derive the RNA (or vice versa) by strandard transcription (or reverse transcription). In addition, additional modifications to the bases can be present. The invention also includes conservative variations of leucyl O-tRNA. For example, conservative variations of leucyl O-tRNA include those molecules that function like the leucyl O-tRNA of any one of SEQ ID NO.: 1-7 and 12 and maintain the tRNA L-shaped structure, but do not have the same sequence (and are other than wild type leucyl tRNA molecules). See also, the section herein entitled “Nucleic acids and Polypeptides Sequence and Variants.”

The composition comprising a leucyl O-tRNA can further include an orthogonal leucyl aminoacyl-tRNA synthetase (leucyl O-RS), where the leucyl O-RS preferentially aminoacylates the leucyl O-tRNA with a selected amino acid (e.g., an unnatural amino acid). In certain embodiments, a composition that includes a leucyl O-tRNA can further include a translation system (e.g., in vitro or in vivo). A nucleic acid that comprises a polynucleotide that encodes a polypeptide of interest, where the polynucleotide comprises a selector codon that is recognized by the leucyl O-tRNA, or a combination of one or more of these can also be present in the cell. See also, the section herein entitled “Orthogonal aminoacyl-tRNA synthetases.”

Methods of producing an orthogonal tRNA (O-tRNA), e.g., a leucyl O-tRNA, are also a feature of the invention. An O-tRNA, e.g., a leucyl O-tRNA, produced by the method is also a feature of the invention. For example, a method includes mutating an anticodon loop of members of a pool of tRNAs (e.g., a pool of leucyl tRNAs) to allow recognition of a selector codon (e.g., an amber codon, an opal codon, a four base codon, etc.), thereby providing a plurality of potential O-tRNAs; and analyzing secondary structure of a member of the plurality potential O-tRNA to identify non-canonical base pairs in the secondary structure, and optionally mutating the non-canonical base pairs (e.g., the non-canonical base pairs are mutated to canonical base pairs). The non-canonical base pairs can be located in stem region of the secondary structure. Typically, a leucyl O-tRNA possesses an improvement of orthogonality for a desired organism compared to the starting material, e.g., the plurality of tRNA sequences, while preserving its affinity towards a desired RS.

The methods optionally include analyzing the homology of sequences of tRNAs and/or aminoacyl-tRNA synthetases to determine potential candidates for an O-tRNA, O-RS and/or pairs thereof, that appear to be orthogonal for a specific organism. Computer programs known in the art and described herein can be used for the analysis. In one example, to choose potential orthogonal translational components for use in E. coli, a prokaryotic organism, a synthetase and/or a tRNA is chosen that does not display unusual homology to prokaryotic organisms.

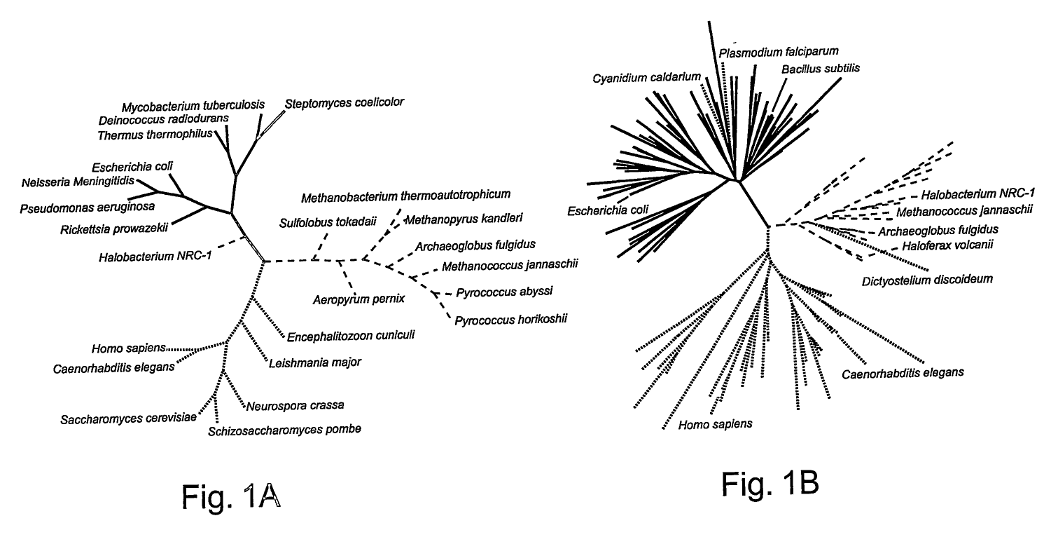

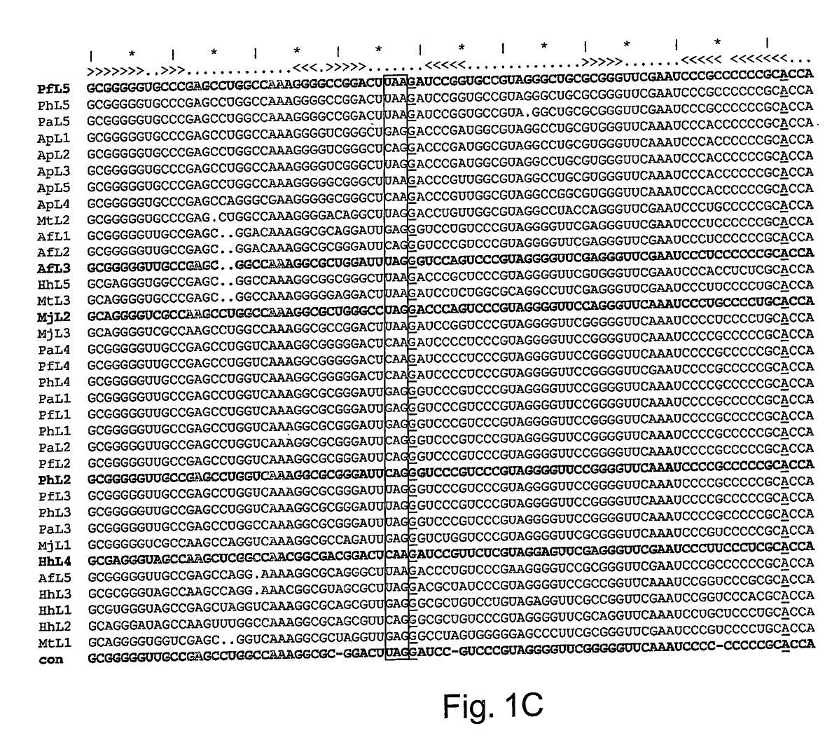

The pool of tRNAs can also be produced by a consensus strategy. For example, the pool of tRNAs is produced by aligning a plurality of tRNA sequences (see e.g., FIG. 1, Panel C); determining a consensus sequence (see e.g., FIG. 1, Panel C); and generating a library of tRNAs using at least a portion, most of, or the entire consensus sequence. For example, a consensus sequence can be compiled with a computer program, e.g., the GCG program pileup. Optionally, degenerate positions determined by the program are changed to the most frequent base at those positions. A library is synthesized by techniques known in the art using the consensus sequence. For example, overlap extension of oligonucleotides in which each site of the tRNA gene can be synthesized as a doped mixture of 90% the consensus sequence and 10% a mixture of the other 3 bases can be used to provide the library based on the consensus sequence. Other mixtures can also be used, e.g., 75% the consensus sequence and 25% a mixture of the other 3 bases, 80% the consensus sequence and 20% a mixture of the other 3 bases, 95% the consensus sequence and 5% a mixture of the other 3 bases, etc.

The library of mutant tRNAs can be generated using various mutagenesis techniques known in the art. For example, the mutant tRNAs can be generated by site-specific mutations, random point mutations, homologous recombination, DNA shuffling or other recursive mutagenesis methods, chimeric construction or any combination thereof.

Additional mutations can be introduced at a specific position(s), e.g., at a nonconservative position(s), or at a conservative position, at a randomized position(s), or a combination of both in a desired loop or region of a tRNA, e.g., an anticodon loop, the acceptor stem, D arm or loop, variable loop, TψC arm or loop, other regions of the tRNA molecule, or a combination thereof. Typically, mutations in a leucyl tRNA include introducing a CU(X)ₙ XXXAA sequence into the anticodon loop, where X refers to any nucleotide, and (X)ₙ is optionally present. The n refers to number of bases the anticodon loop needs to be extended based on the selector codon, e.g., an extended codon, such as a four-, five-, six-base pair, etc. In one embodiment, mutations include matched base pairs in the stem region. In one embodiment, mutations include a first pair selected from T28:A42, G28:C42; C28:G42, etc. and a second pair selected from G49:C65 or C49:G65. The numbering refers to the positions on a tRNA molecule, e.g., see FIG. 4, Panel A. The method can further include adding an additional sequence (CCA) to 3′ terminus of the O-tRNA and/or measuring suppression activity.

Typically, an O-tRNA is obtained by subjecting to negative selection a population of cells of a first species, where the cells comprise a member of the plurality of potential O-tRNAs. The negative selection eliminates cells that comprise a member of the plurality of potential O-tRNAs that is aminoacylated by an aminoacyl-tRNA synthetase (RS) that is endogenous to the cells. This provides a pool of tRNAs that are orthogonal to the cell of the first species.

In certain embodiment in the negative selection, a selector codon(s) is introduced into polynucleotide that encodes a negative selection marker, e.g., an enzyme that confers antibiotic resistance, e.g., β-lactamase, an enzyme that confers a detectable product, e.g., β-galactosidase, chloramphenicol acetyltransferase (CAT), e.g., a toxic product, such as barnase, at a nonessential position, etc. Screening/selection can be done by growing the population of cells in the presence of a selective agent (e.g., an antibiotic, such as ampicillin). In one embodiment, the concentration of the selection agent is varied.

For example, to measure the activity of suppressor leucyl tRNAs, a selection system is used that is based on the in vivo suppression of selector codon, e.g., nonsense or frameshift mutations introduced into a polynucleotide that encodes a negative selection marker, e.g., a gene for β3-lactamase (bla). For example, polynucleotide variants, e.g., bla variants, with, e.g., TAG, AGGA, and TGA, at a certain position (e.g., A184), are constructed. Cells, e.g., bacteria, are transformed with these polynucleotides. In the case of an orthogonal leucyl tRNA, which cannot be efficiently charged by endogenous E. coli synthetases, antibiotic resistance, e.g., ampicillin resistance, should be about or less than that for a bacteria transformed with no plasmid. If the leucyl tRNA is not orthogonal, or if a heterologous synthetase capable of charging the tRNA is co-expressed in the system, a higher level of antibiotic, e.g., ampicillin, resistance is be observed. Cells, e.g., bacteria, are chosen that are unable to grow on LB agar plates with antibiotic concentrations about equal to cells transformed with no plasmids.

In the case of a toxic product (e.g., ribonuclease or barnase), when a member of the plurality of potential leucyl tRNAs is aminoacylated by endogenous host, e.g., Escherichia coli synthetases (i.e., it is not orthogonal to the host, e.g., Escherichia coli synthetases), the selector codon is suppressed and the toxic polynucleotide product produced leads to cell death. Cells harboring orthogonal leucyl tRNAs or non-functional tRNAs survive.

In one embodiment, the pool of tRNAs that are orthogonal to a desired organism are then subjected to a positive selection in which a selector codon is placed in a positive selection marker, e.g., encoded by a drug resistance gene, such a β-lactamase gene. The positive selection is performed on cell comprising a polynucleotide encoding or comprising a member of the pool of tRNAs, a polynucleotide encoding a positive selection marker, and a polynucleotide encoding a cognate RS. These polynucleotides are expressed in the cell and the cell is grown in the presence of a selection agent, e.g., ampicillin. Leucyl tRNAs are then selected for their ability to be aminoacylated by the coexpressed cognate synthetase and to insert an amino acid in response to this selector codon. Typically, these cells show an enhancement in suppression efficiency compared to cells harboring non-functional tRNA(s), or tRNAs that cannot efficiently be recognized by the synthetase of interest. The cell harboring the non-functional tRNAs or tRNAs that are not efficiently recognized by the synthetase of interest, are sensitive to the antibiotic. Therefore, leucyl tRNAs that: (i) are not substrates for endogenous host, e.g., Escherichia coli, synthetases; (ii) can be aminoacylated by the synthetase of interest; and (iii) are functional in translation, survive both selections.

The stringency of the selection, e.g., the positive selection, the negative selection or both the positive and negative selection, in the above described-methods, optionally include varying the selection stringency. For example, because barnase is an extremely toxic protein, the stringency of the negative selection can be controlled by introducing different numbers of selector codons into the barnase gene and/or by using an inducible promoter. In another example, the concentration of the selection or screening agent is varied (e.g., ampicillin concentration). In one aspect of the invention, the stringency is varied because the desired activity can be low during early rounds. Thus, less stringent selection criteria are applied in early rounds and more stringent criteria are applied in later rounds of selection. In certain embodiments, the negative selection, the positive selection or both the negative and positive selection, can be repeated multiple times. Multiple different negative selection markers, positive selection markers or both negative and positive selection markers, can be used. In certain embodiments, the positive and negative selection marker can be the same.

Other types of selections/screening can be used in the invention for producing orthogonal translational components, e.g., a leucyl O-tRNA, a leucyl O-RS, and a leucyl O-tRNA/O-RS pair. For example, the negative selection marker, the positive selection marker or both the positive and negative selection markers can include a marker that fluoresces or catalyzes a luminescent reaction in the presence of a suitable reactant. In another embodiment, a product of the marker is detected by fluorescence-activated cell sorting (FACS) or by luminescence. Optionally, the marker includes an affinity based screening marker. See, Francisco, J. A., et al., (1993) Production and fluorescence-activated cell sorting of Escherichia coli expressing a functional antibody fragment on the external surface. Proc Natl Acad Sci USA. 90:10444-8.

Additional general methods for producing a recombinant orthogonal tRNA can be found, e.g., in International patent applications WO 2002/086075, entitled “Methods and compositions for the production of orthogonal tRNA-aminoacyltRNA synthetase pairs;” and, International Application Number PCT/2004/011786, filed Apr. 16, 2004, entitled “EXPANDING THE EUKARYOTIC GENETIC CODE.” See also, Forster et al., (2003) Programming peptidomimetic synthetases by translating genetic codes designed de novo PNAS 100(11):6353-6357; and, Feng et al., (2003), Expanding tRNA recognition of a tRNA synthetase by a single amino acid change, PNAS 100(10): 5676-5681. These are applied to the present invention, e.g., using the substrates (e.g., leucyl-O-tRNAs or O-RSs) in such available selection methods.

Orthogonal aminoacyl-tRNA Synthetase (O-RS)

A leucyl O-RS of the invention preferentially aminoacylates a leucyl O-tRNA with a selected amino acid in vitro or in vivo. A leucyl O-RS of the invention can be provided to the translation system, e.g., a cell, by a polypeptide that includes a leucyl O-RS and/or by a polynucleotide that encodes a leucyl O-RS or a portion thereof. For example, a leucyl O-RS, or a portion thereof, is encoded by a polynucleotide sequence as set forth in any one of SEQ ID NO.: 13-14, or a complementary polynucleotide sequence thereof. In another example, a leucyl O-RS comprises an amino acid sequence as set forth in any one of SEQ ID NO.: 15-16, or a conservative variation thereof. See, e.g., Table 3 and Example 2 herein for sequences of exemplary leucyl O-RS molecules.

Methods for identifying an orthogonal aminoacyl-tRNA synthetase (O-RS), e.g., a leucyl O-RS, for use with an O-tRNA, e.g., a leucyl O-tRNA, are also a feature of the invention. For example, a method includes subjecting to positive selection a population of cells of a first species, where the cells individually comprise: 1) a member of a plurality of aminoacyl-tRNA synthetases (RSs), where the plurality of RSs comprise mutant RSs, RSs derived from a species other than the first species or both mutant RSs and RSs derived from a species other than the first species; 2) the orthogonal tRNA (O-tRNA) from a second species; and 3) a polynucleotide that encodes a positive selection marker and comprises at least one selector codon. Cells are selected or screened for those that show an enhancement in suppression efficiency compared to cells lacking or with a reduced amount of the member of the plurality of RSs. Cells having an enhancement in suppression efficiency comprise an active RS that aminoacylates the O-tRNA. A level of aminoacylation (in vitro or in vivo) by the active RS of a first set of tRNAs from the first species is compared to the level of aminoacylation (in vitro or in vivo) by the active RS of a second set of tRNAs from the second species. The level of aminoacylation can be determined by a detectable substance (e.g., a labeled amino acid or unnatural amino acid). The active RS that more efficiently aminoacylates the second set of tRNAs compared to the first set of tRNAs is selected, thereby providing an efficient (optimized) orthogonal aminoacyl-tRNA synthetase for use with the O-tRNA. An O-RS, e.g., a leucyl O-RS, identified by the method, is also a feature of the invention.

Any of a number of assays can be used to determine aminoacylation. These assays can be performed in vitro or in vivo. For example, in vitro aminoacylation assays are described in, e.g., Hoben, P., and Soll, D. (1985) Methods Enzymol. 113:55-59. Aminoacylation can also be determined by using a reporter along with orthogonal translation components and detecting the reporter in a cell expressing a polynucleotide comprising at least one selector codon that encodes a protein. See also, WO 2002/085923, entitled “IN VIVO INCORPORATION OF UNNATURAL AMINO ACIDS;” and, U.S. Ser. No. 60/479,931 entitled “EXPANDING THE EUKARYOTIC GENETIC CODE.”

Identified leucyl O-RS can be further manipulated to alter the substrate specificity of the synthetase, so that only a desired unnatural amino acid, but not any of the common 20 amino acids are charged to the leucyl O-tRNA. Methods to generate an orthogonal leucyl aminoacyl tRNA synthetase with a substrate specificity for an unnatural amino acid include mutating the synthetase, e.g., at the active site in the synthetase, at the editing mechanism site in the synthetase, at different sites by combining different domains of synthetases, or the like, and applying a selection process. A strategy is used, which is based on the combination of a positive selection followed by a negative selection. In the positive selection, suppression of the selector codon introduced at a nonessential position(s) of a positive marker allows cells to survive under positive selection pressure. In the presence of both natural and unnatural amino acids, survivors thus encode active synthetases charging the orthogonal suppressor tRNA with either a natural or unnatural amino acid. In the negative selection, suppression of a selector codon introduced at a nonessential position(s) of a negative marker removes synthetases with natural amino acid specificities. Survivors of the negative and positive selection encode synthetases that aminoacylate (charge) the orthogonal suppressor tRNA with unnatural amino acids only. These synthetases can then be subjected to further mutagenesis, e.g., DNA shuffling or other recursive mutagenesis methods.

The library of mutant leucyl O-RSs can be generated using various mutagenesis techniques known in the art. For example, the mutant RSs can be generated by site-specific mutations, random point mutations, homologous recombination, DNA shuffling or other recursive mutagenesis methods, chimeric construction or any combination thereof. For example, a library of mutant leucyl RSs can be produced from two or more other, e.g., smaller, less diverse “sub-libraries.” Chimeric libraries of RSs are also included in the invention. It should be noted that libraries of tRNA synthetases from various organism (e.g., microorganisms such as eubacteria or archaebacteria) such as libraries that comprise natural diversity (see, e.g., U.S. Pat. No. 6,238,884 to Short et al; U.S. Pat. No. 5,756,316 to Schallenberger et al; U.S. Pat. No. 5,783,431 to Petersen et al; U.S. Pat. No. 5,824,485 to Thompson et al; U.S. Pat. No. 5,958,672 to Short et al), are optionally constructed and screened for orthogonal pairs.

Once the synthetases are subject to the positive and negative selection/screening strategy, these synthetases can then be subjected to further mutagenesis. For example, a nucleic acid that encodes the leucyl O-RS can be isolated; a set of polynucleotides that encode mutated leucyl O-RSs (e.g., by random mutagenesis, site-specific mutagenesis, recombination or any combination thereof) can be generated from the nucleic acid; and, these individual steps or a combination of these steps can be repeated until a mutated leucyl O-RS is obtained that preferentially aminoacylates the leucyl O-tRNA with the unnatural amino acid. In one aspect of the invention, the steps are performed multiple times, e.g., at least two times.

Additional levels of selection/screening stringency can also be used in the methods of the invention, for producing leucyl O-tRNA, leucyl O-RS, or pairs thereof. The selection or screening stringency can be varied on one or both steps of the method to produce an O-RS. This could include, e.g., varying the amount of selection/screening agent that is used, etc. Additional rounds of positive and/or negative selections can also be performed. Selecting or screening can also comprise one or more positive or negative selection or screening that includes, e.g., a change in amino acid permeability, a change in translation efficiency, a change in translational fidelity, etc. Typically, the one or more change is based upon a mutation in one or more gene in an organism in which an orthogonal tRNA-tRNA synthetase pair is used to produce protein.

Additional general details for producing O-RS, and altering the substrate specificity of the synthetase can be found in WO 2002/086075 entitled “Methods and compositions for the production of orthogonal tRNA-aminoacyltRNA synthetase pairs;” and International Application Number PCT/US2004/011786, filed Apr. 16, 2004.

Source and Host Organisms

The translational components of the invention can be derived from non-eukaryotic organisms. For example, the orthogonal O-tRNA can be derived from a non-eukaryotic organism, e.g., an archaebacterium, such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, or the like, or a eubacterium, such as Escherichia coli, Thermus thermophilus, Bacillus stearothermphilus, or the like, while the orthogonal O-RS can be derived from a non-eukaryotic organism, e.g., Methanobacterium thermoautotrophicum, Halobacterium such as Haloferax volcanii and Halobacterium species NRC-1, Archaeoglobus fulgidus, Pyrococcus fulgidsus, Pyrococcus horikoshii, Aeuropyrum pernix, or the like, or a eubacterium, such as Escherichia coli, Thermus thermophilus, Bacillus stearothermphilus, or the like. In one embodiment, eukaryotic sources can also be used, e.g., plants, algae, protists, fungi, yeasts, animals (e.g., mammals, insects, arthropods, etc.), or the like.

The individual components of a leucyl O-tRNA/O-RS pair can be derived from the same organism or different organisms. In one embodiment, the leucyl O-tRNA/O-RS pair is from the same organism. Alternatively, the leucyl O-tRNA and the leucyl O-RS of the leucyl O-tRNA/O-RS pair are from different organisms. For example, the leucyl O-tRNA can be derived from, e.g., a Halobacterium sp NRC-1, and the leucyl O-RS can be derived from, e.g., a Methanobacterium thermoautrophicum.

The leucyl O-tRNA, leucyl O-RS or leucyl O-tRNA/O-RS pair can be selected or screened in vivo or in vitro and/or used in a cell, e.g., a non-eukaryotic cells (such as E. coli cell), or a eukaryotic cell, to produce a polypeptide with a selected amino acid (e.g., an unnatural amino acid). A non-eukaryotic cell can be from a variety of sources, e.g., Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, or the like, or a eubacterium, such as Escherichia coli, Thermus thermophilus, Bacillus stearothermphilus, or the like. A eukaryotic cell can be from any of a variety of sources, e.g., a plant (e.g., complex plant such as monocots, or dicots), an algae, a protist, a fungus, a yeast (e.g., Saccharomyces cerevisiae), an animal (e.g., a mammal, an insect, an arthropod, etc.), or the like. Compositions of cells with translational components of the invention are also a feature of the invention.

See also, International Application Number PCT/US2004/011786, filed Apr. 16, 2004, entitled “Expanding the Eukaryotic Genetic Code” for screening O-tRNA and/or O-RS in one species for use in another species.

Selector Codons

Selector codons of the invention expand the genetic codon framework of protein biosynthetic machinery. For example, a selector codon includes, e.g., a unique three base codon, a nonsense codon, such as a stop codon, e.g., an amber codon (UAG), or an opal codon (UGA), an unnatural codon, at least a four base codon, a rare codon, or the like. A number of selector codons can be introduced into a desired gene, e.g., one or more, two or more, more than three, etc.

In one embodiment, the methods involve the use of a selector codon that is a stop codon for the incorporation of a selected amino acid, e.g., an unnatural amino acids, in vivo in a cell. For example, a leucyl O-tRNA is produced that recognizes the stop codon and is aminoacylated by a leucyl O-RS with a selected amino acid. This leucyl O-tRNA is not recognized by the naturally occurring host's aminoacyl-tRNA synthetases. Conventional site-directed mutagenesis can be used to introduce the stop codon at the site of interest in a polypeptide of interest. See, e.g., Sayers, J. R., et al. (1988), 5′,3′ Exonuclease in phosphorothioate-based oligonucleotide-directed mutagenesis. Nucleic Acids Res, 791-802. When the leucyl O-RS, leucyl O-tRNA and the nucleic acid that encodes a polypeptide of interest are combined, e.g., in vivo, the selected amino acid is incorporated in response to the stop codon to give a polypeptide containing the selected amino acid, e.g., an unnatural amino acid, at the specified position. In one embodiment of the invention, a stop codon used as a selector codon is an amber codon, UAG, and/or an opal codon, UGA. For example, see SEQ ID NO: 3 for an example of a leucyl O-tRNA that recognizes an amber codon, and see SEQ ID NO: 7 for an example of a leucyl O-tRNA that recognizes an opal codon. A genetic code in which UAG and UGA are both used as a selector codon can encode 22 amino acids while preserving the ochre nonsense codon, UAA, which is the most abundant termination signal.

The incorporation of selected amino acids, e.g., unnatural amino acids, in vivo, can be done without significant perturbation of the host cell. For example, in non-eukaryotic cells, such as Escherichia coli, because the suppression efficiency for the UAG codon depends upon the competition between the O-tRNA, e.g., the amber suppressor tRNA, and the release factor 1 (RF1) (which binds to the UAG codon and initiates release of the growing peptide from the ribosome), the suppression efficiency can be modulated by, e.g., either increasing the expression level of O-tRNA, e.g., the suppressor tRNA, or using an RF1 deficient strain. In eukaryotic cells, because the suppression efficiency for the UAG codon depends upon the competition between the O-tRNA, e.g., the amber suppressor tRNA, and a eukaryotic release factor (e.g., eRF) (which binds to a stop codon and initiates release of the growing peptide from the ribosome), the suppression efficiency can be modulated by, e.g., increasing the expression level of O-tRNA, e.g., the suppressor tRNA.

Unnatural amino acids can also be encoded with rare codons. For example, when the arginine concentration in an in vitro protein synthesis reaction is reduced, the rare arginine codon, AGG, has proven to be efficient for insertion of Ala by a synthetic tRNA acylated with alanine. See, e.g., Ma et al., Biochemistry, 32:7939 (1993). In this case, the synthetic tRNA competes with the naturally occurring tRNAArg, which exists as a minor species in Escherichia coli. Some organisms do not use all triplet codons. An unassigned codon AGA in Micrococcus luteus has been utilized for insertion of amino acids in an in vitro transcription/translation extract. See, e.g., Kowal and Oliver, Nucl. Acid. Res., 25:4685 (1997). Components of the present invention can be generated to use these rare codons in vivo.

Selector codons can also comprise extended codons, e.g., four or more base codons, such as, four, five, six or more base codons. Examples of four base codons include, e.g., AGGA, CUAG, UAGA, CCCU, and the like. Examples of five base codons include, e.g., AGGAC, CCCCU, CCCUC, CUAGA, CUACU, UAGGC, and the like. Methods of the invention include using extended codons based on frameshift suppression. Four or more base codons can insert, e.g., one or multiple selected amino acids, e.g., unnatural amino acids, into the same protein. For example, in the presence of mutated leucyl O-tRNAs, e.g., a special frameshift suppressor tRNAs, with anticodon loops, e.g., with a CU(X)ₙ XXXAA sequence (where n=1), the four or more base codon is read as single amino acid. For example, see SEQ ID NOs.: 6 and 12 for leucyl O-tRNAs that recognize a four base codon. In other embodiments, the anticodon loops can decode, e.g., at least a four-base codon, at least a five-base codon, or at least a six-base codon or more. Since there are 256 possible four-base codons, multiple unnatural amino acids can be encoded in the same cell using a four or more base codon. See also, Anderson et al., (2002) Exploring the Limits of Codon and Anticodon Size, Chemistry and Biology, 9:237-244; Magliery, (2001) Expanding the Genetic Code: Selection of Efficient Suppressors of Four-base Codons and Identification of “Shifty” Four-base Codons with a Library Approach in Escherichia coli, J. Mol. Biol. 307: 755-769.

For example, four-base codons have been used to incorporate unnatural amino acids into proteins using in vitro biosynthetic methods. See, e.g., Ma et al., (1993) Biochemistry, 32:7939; and Hohsaka et al., (1999) J. Am. Chem. Soc. 121:34. CGGG and AGGU were used to simultaneously incorporate 2-naphthylalanine and an NBD derivative of lysine into streptavidin in vitro with two chemically acylated frameshift suppressor tRNAs. See, e.g., Hohsaka et al., (1999) J. Am. Chem. Soc., 121:12194. In an in vivo study, Moore et al. examined the ability of tRNALeu derivatives with NCUA anticodons to suppress UAGN codons (N can be U, A, G, or C), and found that the quadruplet UAGA can be decoded by a tRNALeu with a UCUA anticodon with an efficiency of 13 to 26% with little decoding in the 0 or −1 frame. See, Moore et al., (2000) J. Mol. Biol. 298:195. In one embodiment, extended codons based on rare codons or nonsense codons can be used in invention, which can reduce missense readthrough and frameshift suppression at other unwanted sites.

For a given system, a selector codon can also include one of the natural three base codons, where the endogenous system does not use (or rarely uses) the natural base codon. For example, this includes a system that is lacking a tRNA that recognizes the natural three base codon, and/or a system where the three base codon is a rare codon.

Selector codons optionally include unnatural base pairs. These unnatural base pairs further expand the existing genetic alphabet. One extra base pair increases the number of triplet codons from 64 to 125. Properties of third base pairs include stable and selective base pairing, efficient enzymatic incorporation into DNA with high fidelity by a polymerase, and the efficient continued primer extension after synthesis of the nascent unnatural base pair. Descriptions of unnatural base pairs which can be adapted for methods and compositions include, e.g., Hirao, et al., (2002) An unnatural base pair for incorporating amino acid analogues into protein, Nature Biotechnology, 20:177-182. See, also, Wu, Y., et al., (2002) J. Am. Chem. Soc. 124:14626-14630. Other relevant publications are listed herein.

For in vivo usage, the unnatural nucleoside is membrane permeable and is phosphorylated to form the corresponding triphosphate. In addition, the increased genetic information is stable and not destroyed by cellular enzymes. Previous efforts by Benner and others took advantage of hydrogen bonding patterns that are different from those in canonical Watson-Crick pairs, the most noteworthy example of which is the iso-C:iso-G pair. See, e.g., Switzer et al., (1989) J. Am. Chem. Soc., 111:8322; and Piccirilli et al., (1990) Nature, 343:33; Kool, (2000) Curr. Opin. Chem. Biol., 4:602. These bases in general mispair to some degree with natural bases and cannot be enzymatically replicated. Kool and co-workers demonstrated that hydrophobic packing interactions between bases can replace hydrogen bonding to drive the formation of base pair. See, Kool, (2000) Curr. Opin. Chem. Biol., 4:602; and Guckian and Kool, (1998) Angew. Chem. Int. Ed. Engl., 36, 2825. In an effort to develop an unnatural base pair satisfying all the above requirements, Schultz, Romesberg and co-workers have systematically synthesized and studied a series of unnatural hydrophobic bases. A PICS:PICS self-pair is found to be more stable than natural base pairs, and can be efficiently incorporated into DNA by Klenow fragment of Escherichia coli DNA polymerase I (KF). See, e.g., McMinn et al., (1999) J. Am. Chem. Soc., 121:11586; and Ogawa et al., (2000) J. Am. Chem. Soc., 122:3274. A 3MN:3MN self-pair can be synthesized by KF with efficiency and selectivity sufficient for biological function. See, e.g., Ogawa et al., (2000) J. Am. Chem. Soc., 122:8803. However, both bases act as a chain terminator for further replication. A mutant DNA polymerase has been recently evolved that can be used to replicate the PICS self pair. In addition, a 7AI self pair can be replicated. See, e.g., Tae et al., (2001) J. Am. Chem. Soc., 123:7439. A novel metallobase pair, Dipic:Py, has also been developed, which forms a stable pair upon binding Cu(II). See, Meggers et al., (2000) J. Am. Chem. Soc., 122:10714. Because extended codons and unnatural codons are intrinsically orthogonal to natural codons, the methods of the invention can take advantage of this property to generate orthogonal tRNAs for them.

A translational bypassing system can also be used to incorporate a selected amino acid, e.g., an unnatural amino acid, in a desired polypeptide. In a translational bypassing system, a large sequence is inserted into a gene but is not translated into protein. The sequence contains a structure that serves as a cue to induce the ribosome to hop over the sequence and resume translation downstream of the insertion.

Selected and Unnatural Amino Acids

As used herein, a selected amino acid refers to any desired naturally occurring amino acid or unnatural amino acid. A naturally occurring amino acid includes any one of the twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine. In one embodiment, the selected amino acid is incorporated into a growing polypeptide chain with high fidelity, e.g., at greater than 75% efficiency for a given selector codon, at greater than about 80% efficiency for a given selector codon, at greater than about 90% efficiency for a given selector codon, at greater than about 95% efficiency for a given selector codon, or at greater than about 99% or more efficiency for a given selector codon.

As used herein, an unnatural amino acid refers to any amino acid, modified amino acid, or amino acid analogue other than selenocysteine and/or pyrrolysine and the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine. The generic structure of an alpha-amino acid is illustrated by Formula I:

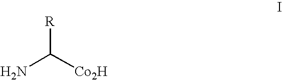

An unnatural amino acid is typically any structure having Formula I wherein the R group is any substituent other than one used in the twenty natural amino acids. See, e.g., Biochemistry by L. Stryer, 3^{rd} ed. 1988, Freeman and Company, New York, for structures of the twenty natural amino acids. Note that, the unnatural amino acids of the invention can be naturally occurring compounds other than the twenty alpha-amino acids above.

Because the unnatural amino acids of the invention typically differ from the natural amino acids in side chain only, the unnatural amino acids form amide bonds with other amino acids, e.g., natural or unnatural, in the same manner in which they are formed in naturally occurring proteins. However, the unnatural amino acids have side chain groups that distinguish them from the natural amino acids.

Because the unnatural amino acids of the invention typically differ from the natural amino acids in side chain, the unnatural amino acids form amide bonds with other amino acids, e.g., natural or unnatural, in the same manner in which they are formed in naturally occurring proteins. However, the unnatural amino acids have side chain groups that distinguish them from the natural amino acids. For example, R in Formula I optionally comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amine, and the like, or any combination thereof. In some embodiments, the unnatural amino acids have a photoactivatable cross-linker that is used, e.g., to link a protein to a solid support. In one embodiment, the unnatural amino acids have a saccharide moiety attached to the amino acid side chain.

In addition to unnatural amino acids that contain novel side chains, unnatural amino acids also optionally comprise modified backbone structures, e.g., as illustrated by the structures of Formula II and III: 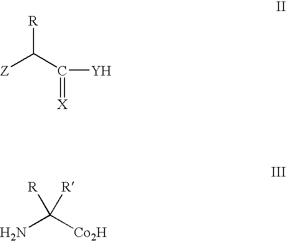
wherein Z typically comprises OH, NH₂, SH, NH—R′, or S—R′; X and Y, which can be the same or different, typically comprise S or O, and R and R′, which are optionally the same or different, are typically selected from the same list of constituents for the R group described above for the unnatural amino acids having Formula I as well as hydrogen. For example, unnatural amino acids of the invention optionally comprise substitutions in the amino or carboxyl group as illustrated by Formulas II and III. Unnatural amino acids of this type include, but are not limited to, α-hydroxy acids, α-thioacids α-aminothiocarboxylates, e.g., with side chains corresponding to the common twenty natural amino acids or unnatural side chains. In addition, substitutions at the α-carbon optionally include L, D, or α-α-disubstituted amino acids such as D-glutamate, D-alanine, D-methyl-O-tyrosine, aminobutyric acid, and the like. Other structural alternatives include cyclic amino acids, such as proline analogues as well as 3, 4, 6, 7, 8, and 9 membered ring proline analogues, β and γ amino acids such as substituted β-alanine and γ-amino butyric acid.

For example, many unnatural amino acids are based on natural amino acids, such as tyrosine, glutamine, phenylalanine, and the like. Tyrosine analogs include para-substituted tyrosines, ortho-substituted tyrosines, and meta substituted tyrosines, wherein the substituted tyrosine comprises an acetyl group, a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, or the like. In addition, multiply substituted aryl rings are also contemplated. Glutamine analogs of the invention include, but are not limited to, α-hydroxy derivatives, γ-substituted derivatives, cyclic derivatives, and amide substituted glutamine derivatives. Example phenylalanine analogs include, but are not limited to, para-substituted phenylalanines, ortho-substituted phenyalanines, and meta-substituted phenylalanines, wherein the substituent comprises a hydroxy group, a methoxy group, a methyl group, an allyl group, an aldehyde or keto group, or the like. Specific examples of unnatural amino acids include, but are not limited to, a p-acetyl-L-phenylalanine, a p-propargyl-phenylalanine, O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like. The structures of a variety of unnatural amino acids are provided in, for example, FIGS. 16, 17, 18, 19, 26, and 29 of WO 2002/085923 entitled “In vivo incorporation of unnatural amino acids.”

Chemical Synthesis of Unnatural Amino Acids

Many of the unnatural amino acids provided above are commercially available, e.g., from Sigma (USA) or Aldrich (Milwaukee, Wis., USA). Those that are not commercially available are optionally synthesized as provided in various publications or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional publications describing the synthesis of unnatural amino acids include, e.g., WO 2002/085923 entitled “In vivo incorporation of Unnatural Amino Acids;” Matsoukas et al., (1995) J. Med. Chem., 38, 4660-4669; King, F. E. & Kidd, D. A. A. (1949) A New Synthesis of Glutamine and of γ-Dipeptides of Glutamic Acid from Phthylated Intermediates. J. Chem. Soc., 3315-3319; Friedman, O. M. & Chatterji, R. (1959) Synthesis of Derivatives of Glutamine as Model Substrates for Anti-Tumor Agents. J. Am. Chem. Soc. 81, 3750-3752; Craig, J. C. et al. (1988) Absolute Configuration of the Enantiomers of 7-Chloro-4[[4-(diethylamino)-1-methylbutyl]amino]quinoline (Chloroquine). J. Org. Chem. 53, 1167-1170; Azoulay, M., Vilmont, M. & Frappier, F. (1991) Glutamine analogues as Potential Antimalarials, Eur. J. Med. Chem. 26, 201-5; Koskinen, A. M. P. & Rapoport, H. (1989) Synthesis of 4-Substituted Prolines as Conformationally Constrained Amino Acid Analogues. J. Org. Chem. 54, 1859-1866; Christie, B. D. & Rapoport, H. (1985) Synthesis of Optically Pure Pipecolates front L-Asparagine. Application to the Total Synthesis of (+)-Apovincamine through Amino Acid Decarbonylation and Iminium Ion Cyclization. J. Org. Chem. 1989:1859-1866; Barton et al., (1987) Synthesis of Novel α-Amino-Acids and Derivatives Using Radical Chemistry: Synthesis of L-and D-a-Amino-Adipic Acids, L-a-aminopimelic Acid and Appropriate Unsaturated Derivatives. Tetrahedron Lett. 43:4297-4308; and, Subasinghe et al., (1992) Quisqualic acid analogues: synthesis of beta-heterocyclic 2-aminopropanoic acid derivatives and their activity at a novel quisqualate-sensitized site. J. Med. Chem. 35:4602-7. See also, International Application Number PCT/US03/41346, entitled “Protein Arrays,” filed on Dec. 22, 2003.

Cellular Uptake of Unnatural Amino Acids

Unnatural amino acid uptake by a cell is one issue that is typically considered when designing and selecting unnatural amino acids, e.g., for incorporation into a protein. For example, the high charge density of α-amino acids suggests that these compounds are unlikely to be cell permeable. Natural amino acids are taken up into the cell via a collection of protein-based transport systems often displaying varying degrees of amino acid specificity. A rapid screen can be done which assesses which unnatural amino acids, if any, are taken up by cells. See, e.g., the toxicity assays in, e.g., International Application Number PCT/US03/41346, entitled “Protein Arrays,” filed on Dec. 22, 2003; and Liu, D. R. & Schultz, P. G. (1999) Progress toward the evolution of an organism with an expanded genetic code. PNAS United States 96:4780-4785. Although uptake is easily analyzed with various assays, an alternative to designing unnatural amino acids that are amenable to cellular uptake pathways is to provide biosynthetic pathways to create amino acids in vivo.

Biosynthesis of Unnatural Amino Acids

Many biosynthetic pathways already exist in cells for the production of amino acids and other compounds. While a biosynthetic method for a particular unnatural amino acid may not exist in nature, e.g., in a cell, the invention provides such methods. For example, biosynthetic pathways for unnatural amino acids are optionally generated in host cell by adding new enzymes or modifying existing host cell pathways. Additional new enzymes are optionally naturally occurring enzymes or artificially evolved enzymes. For example, the biosynthesis of p-aminophenylalanine (as presented in an example in WO 2002/085923, supra) relies on the addition of a combination of known enzymes from other organisms. The genes for these enzymes can be introduced into a cell by transforming the cell with a plasmid comprising the genes. The genes, when expressed in the cell, provide an enzymatic pathway to synthesize the desired compound. Examples of the types of enzymes that are optionally added are provided in the examples below. Additional enzymes sequences are found, e.g., in Genbank. Artificially evolved enzymes are also optionally added into a cell in the same manner. In this manner, the cellular machinery and resources of a cell are manipulated to produce unnatural amino acids.

Indeed, any of a variety of methods can be used for producing novel enzymes for use in biosynthetic pathways, or for evolution of existing pathways, for the production of unnatural amino acids, in vitro or in vivo. Many available methods of evolving enzymes and other biosynthetic pathway components can be applied to the present invention to produce unnatural amino acids (or, indeed, to evolve synthetases to have new substrate specificities or other activities of interest). For example, DNA shuffling is optionally used to develop novel enzymes and/or pathways of such enzymes for the production of unnatural amino acids (or production of new synthetases), in vitro or in vivo. See, e.g., Stemmer (1994), Rapid evolution of a protein in vitro by DNA shuffling, Nature 370(4):389-391; and, Stemmer, (1994), DNA shuffling by random fragmentation and reassembly: In vitro recombination for molecular evolution, Proc. Natl. Acad. Sci. USA., 91:10747-10751. A related approach shuffles families of related (e.g., homologous) genes to quickly evolve enzymes with desired characteristics. An example of such “family gene shuffling” methods is found in Crameri et al. (1998) “DNA shuffling of a family of genes from diverse species accelerates directed evolution” Nature, 391(6664): 288-291. New enzymes (whether biosynthetic pathway components or synthetases) can also be generated using a DNA recombination procedure known as “incremental truncation for the creation of hybrid enzymes” (“ITCHY”), e.g., as described in Ostermeier et al. (1999) “A combinatorial approach to hybrid enzymes independent of DNA homology” Nature Biotech 17:1205. This approach can also be used to generate a library of enzyme or other pathway variants which can serve as substrates for one or more in vitro or in vivo recombination methods. See, also, Ostermeier et al. (1999) “Combinatorial Protein Engineering by Incremental Truncation,” Proc. Natl. Acad. Sci. USA, 96: 3562-67, and Ostermeier et al. (1999), “Incremental Truncation as a Strategy in the Engineering of Novel Biocatalysts,” Biological and Medicinal Chemistry, 7: 2139-44. Another approach uses exponential ensemble mutagenesis to produce libraries of enzyme or other pathway variants that are, e.g., selected for an ability to catalyze a biosynthetic reaction relevant to producing an unnatural amino acid (or a new synthetase). In this approach, small groups of residues in a sequence of interest are randomized in parallel to identify, at each altered position, amino acids which lead to functional proteins. Examples of such procedures, which can be adapted to the present invention to produce new enzymes for the production of unnatural amino acids (or new synthetases) are found in Delegrave & Youvan (1993) Biotechnology Research 11:1548-1552. In yet another approach, random or semi-random mutagenesis using doped or degenerate oligonucleotides for enzyme and/or pathway component engineering can be used, e.g., by using the general mutagenesis methods of e.g., Arkin and Youvan (1992) “Optimizing nucleotide mixtures to encode specific subsets of amino acids for semi-random mutagenesis” Biotechnology 10:297-300; or Reidhaar-Olson et al. (1991) “Random mutagenesis of protein sequences using oligonucleotide cassettes” Methods Enzymol. 208:564-86. Yet another approach, often termed a “non-stochastic” mutagenesis, which uses polynucleotide reassembly and site-saturation mutagenesis can be used to produce enzymes and/or pathway components, which can then be screened for an ability to perform one or more synthetase or biosynthetic pathway function (e.g., for the production of unnatural amino acids in vivo). See, e.g., Short “Non-Stochastic Generation of Genetic Vaccines and Enzymes” WO 00/46344.

An alternative to such mutational methods involves recombining entire genomes of organisms and selecting resulting progeny for particular pathway functions (often referred to as “whole genome shuffling”). This approach can be applied to the present invention, e.g., by genomic recombination and selection of an organism (e.g., an E. coli or other cell) for an ability to produce an unnatural amino acid (or intermediate thereof). For example, methods taught in the following publications can be applied to pathway design for the evolution of existing and/or new pathways in cells to produce unnatural amino acids in vivo: Patnaik et al. (2002) “Genome shuffling of lactobacillus for improved acid tolerance” Nature Biotechnology, 20(7): 707-712; and Zhang et al. (2002) “Genome shuffling leads to rapid phenotypic improvement in bacteria” Nature, February 7, 415(6872): 644-646.

Other techniques for organism and metabolic pathway engineering, e.g., for the production of desired compounds are also available and can also be applied to the production of unnatural amino acids. Examples of publications teaching useful pathway engineering approaches include: Nakamura and White (2003) “Metabolic engineering for the microbial production of 1,3 propanediol” Curr. Opin. Biotechnol. 14(5):454-9; Berry et al. (2002) “Application of Metabolic Engineering to improve both the production and use of Biotech Indigo” J. Industrial Microbiology and Biotechnology 28:127-133; Banta et al. (2002) “Optimizing an artificial metabolic pathway: Engineering the cofactor specificity of Corynebacterium 2,5-diketo-D-gluconic acid reductase for use in vitamin C biosynthesis” Biochemistry, 41(20), 6226-36; Selivonova et al. (2001) “Rapid Evolution of Novel Traits in Microorganisms” Applied and Environmental Microbiology, 67:3645, and many others.

Regardless of the method used, typically, the unnatural amino acid produced with an engineered biosynthetic pathway of the invention is produced in a concentration sufficient for efficient protein biosynthesis, e.g., a natural cellular amount, but not to such a degree as to significantly affect the concentration of other cellular amino acids or to exhaust cellular resources. Typical concentrations produced in vivo in this manner are about 10 mM to about 0.05 mM. Once a cell is engineered to produce enzymes desired for a specific pathway and an unnatural amino acid is generated, in vivo selections are optionally used to further optimize the production of the unnatural amino acid for both ribosomal protein synthesis and cell growth.

As described above and below, the invention provides for nucleic acid polynucleotide sequences and polypeptide amino acid sequences, e.g., leucyl O-tRNAs and leucyl O-RSs, and, e.g., compositions, systems and methods comprising said sequences. Examples of said sequences, e.g., leucyl O-tRNAs and leucyl O-RSs are disclosed herein (see, Table 3, e.g., SEQ ID NO. 1-7, 12-16). However, one of skill in the art will appreciate that the invention is not limited to those sequences disclosed herein, e.g., as in the Examples. One of skill will appreciate that the invention also provides many related and unrelated sequences with the functions described herein, e.g., encoding a leucyl O-tRNA or a leucyl O-RS.

The invention provides polypeptides (leucyl O-RSs) and polynucleotides, e.g., leucyl O-tRNA, polynucleotides that encode leucyl O-RSs or portions thereof, oligonucleotides used to isolate aminoacyl-tRNA synthetase clones, etc. Polynucleotides of the invention include those that encode proteins or polypeptides of interest of the invention with one or more selector codon. In addition, polynucleotides of the invention include, e.g., a polynucleotide comprising a nucleotide sequence as set forth in any one of SEQ ID NO.: 1-2, 4-7 and 12; a polynucleotide that is complementary to or that encodes a polynucleotide sequence thereof. A polynucleotide of the invention also includes a polynucleotide that encodes a polypeptide of the invention. Similarly, a nucleic acid that hybridizes to a polynucleotide indicated above under highly stringent conditions over substantially the entire length of the nucleic acid is a polynucleotide of the invention. In one embodiment, a composition includes a polypeptide of the invention and an excipient (e.g., buffer, water, pharmaceutically acceptable excipient, etc.). The invention also provides an antibody or antisera specifically immunoreactive with a polypeptide of the invention.

A polynucleotide of the invention also includes a polynucleotide that is, e.g., at least 75%, at least 80%, at least 90%, at least 95%, at least 98% or more identical to that of a naturally occurring leucyl tRNA and comprises an anticodon loop comprising a CU(X)ₙ XXXAA sequence, an stem region lacking noncanonical base pairs and a conserved discriminator base at position 73. A polynucleotide also includes a polynucleotide that is, e.g., at least 75%, at least 80%, at least 90%, at least 95%, at least 98% or more identical to that of a naturally occurring leucyl tRNA and comprises an anticodon loop comprising a CUUCCUAA sequence, a first pair selected from T28:A42, G28:C42 and/or C28:G42, and a second pair selected from G:49:C65 or C49:G65, wherein the numbering corresponds to that indicated in FIG. 4, Panel A.

In certain embodiments, a vector (e.g., a plasmid, a cosmid, a phage, a virus, etc.) comprises a polynucleotide of the invention. In one embodiment, the vector is an expression vector. In another embodiment, the expression vector includes a promoter operably linked to one or more of the polynucleotides of the invention. In another embodiment, a cell comprises a vector that includes a polynucleotide of the invention.

One of skill will also appreciate that many variants of the disclosed sequences are included in the invention. For example, conservative variations of the disclosed sequences that yield a functionally identical sequence are included in the invention. Variants of the nucleic acid polynucleotide sequences, wherein the variants hybridize to at least one disclosed sequence, are considered to be included in the invention. Unique subsequences of the sequences disclosed herein, as determined by, e.g., standard sequence comparison techniques, are also included in the invention.

Conservative Variations

Owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence are substituted with different amino acids with highly similar properties, are also readily identified as being highly similar to a disclosed construct. Such conservative variations of each disclosed sequence are a feature of the present invention.

“Conservative variations” of a particular nucleic acid sequence refers to those nucleic acids which encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid. Thus, “conservative variations” of a listed polypeptide sequence of the present invention include substitutions of a small percentage, typically less than 5%, more typically less than 2% or 1%, of the amino acids of the polypeptide sequence, with a conservatively selected amino acid of the same conservative substitution group. Finally, the addition of sequences which do not essentially alter the encoded activity of a nucleic acid molecule, such as the addition of a non-functional sequence, is a conservative variation of the basic nucleic acid.

Conservative substitution tables providing functionally similar amino acids are well known in the art. The following sets forth example groups which contain natural amino acids that include “conservative substitutions” for one another.

Conservative Substitution Groups

| Nonpolar and/or | | | Positively | Negatively |
|---|---|---|---|---|
| Aliphatic Side | Polar, Uncharged | Aromatic | Charged Side | Charged Side |
| Chains | Side Chains | Side Chains | Chains | Chains |
| Glycine | Serine | Phenylalanine | Lysine | Aspartate |
| Alanine | Threonine | Tyrosine | Arginine | Glutamate |
| Valine | Cysteine | Tryptophan | Histidine | |
| Leucine | Methionine | | | |
| Isoleucine | Asparagine | | | |
| Proline | Glutamine | | | |

Nucleic Acid Hybridization

Comparative hybridization can be used to identify nucleic acids of the invention, such as SEQ ID NO.: 1-2, 4-7 and 12, including conservative variations of nucleic acids of the invention, and this comparative hybridization method is a preferred method of distinguishing nucleic acids of the invention. In addition, target nucleic acids which hybridize to the nucleic acids represented by SEQ ID NO: 1-2, 4-7 and 12 under high, ultra-high and ultra-ultra high stringency conditions are a feature of the invention. Examples of such nucleic acids include those with one or a few silent or conservative nucleic acid substitutions as compared to a given nucleic acid sequence.

A test nucleic acid is said to specifically hybridize to a probe nucleic acid when it hybridizes at least ½ as well to the probe as to the perfectly matched complementary target, i.e., with a signal to noise ratio at least ½ as high as hybridization of the probe to the target under conditions in which the perfectly matched probe binds to the perfectly matched complementary target with a signal to noise ratio that is at least about 5×-10× as high as that observed for hybridization to any of the unmatched target nucleic acids.

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel, supra. Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis, labeling, detection and quantification of DNA and RNA, including oligonucleotides.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of stringent wash conditions is a 0.2×SSC wash at 65° C. for 15 minutes (see, Sambrook, supra for a description of SSC buffer). Often the high stringency wash is preceded by a low stringency wash to remove background probe signal. An example low stringency wash is 2×SSC at 40° C. for 15 minutes. In general, a signal to noise ratio of 5× (or higher) than that observed for an unrelated probe in the particular hybridization assay indicates detection of a specific hybridization.

“Stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993), supra. and in Hames and Higgins, 1 and 2. Stringent hybridization and wash conditions can easily be determined empirically for any test nucleic acid. For example, in determining highly stringent hybridization and wash conditions, the hybridization and wash conditions are gradually increased (e.g., by increasing temperature, decreasing salt concentration, increasing detergent concentration and/or increasing the concentration of organic solvents such as formalin in the hybridization or wash), until a selected set of criteria are met. For example, the hybridization and wash conditions are gradually increased until a probe binds to a perfectly matched complementary target with a signal to noise ratio that is at least 5× as high as that observed for hybridization of the probe to an unmatched target.

“Very stringent” conditions are selected to be equal to the thermal melting point (Tₘ) for a particular probe. The Tₘ is the temperature (under defined ionic strength and pH) at which 50% of the test sequence hybridizes to a perfectly matched probe. For the purposes of the present invention, generally, “highly stringent” hybridization and wash conditions are selected to be about 5° C. lower than the Tₘ for the specific sequence at a defined ionic strength and pH.

“Ultra high-stringency” hybridization and wash conditions are those in which the stringency of hybridization and wash conditions are increased until the signal to noise ratio for binding of the probe to the perfectly matched complementary target nucleic acid is at least 10× as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or 500× or more as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-ultra-high stringency conditions.

Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

Unique Subsequences

In one aspect, the invention provides a nucleic acid that comprises a unique subsequence in a nucleic acid selected from the sequences of leucyl O-tRNAs and leucyl O-RSs disclosed herein. The unique subsequence is unique as compared to a nucleic acid corresponding to any known leucyl O-tRNA or leucyl O-RS nucleic acid sequence. Alignment can be performed using, e.g., BLAST set to default parameters. Any unique subsequence is useful, e.g., as a probe to identify the nucleic acids of the invention.

Similarly, the invention includes a polypeptide which comprises a unique subsequence in a polypeptide selected from the sequences of leucyl O-RSs disclosed herein. Here, the unique subsequence is unique as compared to a polypeptide corresponding to any of known polypeptide sequence.

The invention also provides for target nucleic acids which hybridizes under stringent conditions to a unique coding oligonucleotide which encodes a unique subsequence in a polypeptide selected from the sequences of leucyl O-RSs wherein the unique subsequence is unique as compared to a polypeptide corresponding to any of the control polypeptides (e.g., parental sequences from which synthetases of the invention were derived, e.g., by mutation). Unique sequences are determined as noted above.

Sequence Comparison Identity, and Homology

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection.

The phrase “substantially identical,” in the context of two nucleic acids or polypeptides (e.g., DNAs encoding an leucyl O-tRNA or leucyl O-RS, or the amino acid sequence of an O-RS) refers to two or more sequences or subsequences that have at least about 60%, about 80%, about 90-95%, about 98%, about 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, or over the full length of the two sequences to be compared.

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, PASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215:403410 (1990). Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Mutagenesis and Other Molecular Biology Techniques

Polynucleotide and polypeptides of the invention and used in the invention can be manipulated using molecular biological techniques. General texts which describe molecular biological techniques include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)). These texts describe mutagenesis, the use of vectors, promoters and many other relevant topics related to, e.g., the generation of genes that include selector codons for production of proteins that include selected amino acids (e.g., unnatural amino acids), leucyl orthogonal tRNAs, leucyl orthogonal synthetases, and pairs thereof.

Various types of mutagenesis are used in the invention, e.g., to mutate tRNA molecules, to produce libraries of leucyl tRNAs, to produce libraries of leucyl synthetases, to insert selector codons that encode a selected amino acid in a protein or polypeptide of interest. They include but are not limited to site-directed, random point mutagenesis, homologous recombination, DNA shuffling or other recursive mutagenesis methods, chimeric construction, mutagenesis using uracil containing templates, oligonucleotide-directed mutagenesis, phosphorothioate-modified DNA mutagenesis, mutagenesis using gapped duplex DNA or the like, or any combination thereof. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present invention. In one embodiment, mutagenesis can be guided by known information of the naturally occurring molecule or altered or mutated naturally occurring molecule, e.g., sequence, sequence comparisons, physical properties, crystal structure or the like.

Host cells are genetically engineered (e.g., transformed, transduced or transfected) with the polynucleotides of the invention or constructs which include a polynucleotide of the invention, e.g., a vector of the invention, which can be, for example, a cloning vector or an expression vector. For example, the coding regions for the orthogonal tRNA, the orthogonal tRNA synthetase, and the protein to be derivatized are operably linked to gene expression control elements that are functional in the desired host cell. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both (e.g., shuttle vectors) and selection markers for both prokaryotic and eukaryotic systems. Vectors are suitable for replication and/or integration in prokaryotes, eukaryotes, or preferably both. See, Giliman & Smith, Gene 8:81 (1979); Roberts, et al., Nature, 328:731 (1987); Schneider, B., et al., Protein Expr. Purif 6435:10 (1995); Ausubel, Sambrook, Berger (all supra). The vector can be, for example, in the form of a plasmid, a bacterium, a virus, a naked polynucleotide, or a conjugated polynucleotide. The vectors are introduced into cells and/or microorganisms by standard methods including electroporation (From et al., Proc. Natl. Acad. Sci. USA 82, 5824 (1985), infection by viral vectors, high velocity ballistic penetration by small particles with the nucleic acid either within the matrix of small beads or particles, or on the surface (Klein et al., Nature 327, 70-73 (1987)), and/or the like.

A catalogue of Bacteria and Bacteriophages useful for cloning is provided, e.g., by the ATCC, e.g., The ATCC Catalogue of Bacteria and Bacteriophage (1992) Gherna et al. (eds) published by the ATCC. Additional basic procedures for sequencing, cloning and other aspects of molecular biology and underlying theoretical considerations are also found in Watson et al. (1992) Recombinant DNA Second Edition Scientific American Books, NY. In addition, essentially any nucleic acid (and virtually any labeled nucleic acid, whether standard or non-standard) can be custom or standard ordered from any of a variety of commercial sources, such as the Midland Certified Reagent Company (Midland, Tex. mcrc.com), The Great American Gene Company (Ramona, Calif. available on the World Wide Web at genco.com), ExpressGen Inc. (Chicago, Ill. available on the World Wide Web at expressgen.com), Operon Technologies Inc. (Alameda, Calif.) and many others.

The engineered host cells can be cultured in conventional nutrient media modified as appropriate for such activities as, for example, screening steps, activating promoters or selecting transformants. These cells can optionally be cultured into transgenic organisms. Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

Proteins and Polypeptides of Interest

Methods of producing a protein in a cell with a selected amino acid at a specified position are also a feature of the invention. For example, a method includes growing, in an appropriate medium, the cell, where the cell comprises a nucleic acid that comprises at least one selector codon and encodes a protein; and, providing the selected amino acid; where the cell further comprises: an orthogonal leucyl-tRNA (leucyl-O-tRNA) that functions in the cell and recognizes the selector codon; and, an orthogonal aminoacyl-tRNA synthetase (O-RS) that preferentially aminoacylates the leucyl-O-tRNA with the selected amino acid. Typically, the leucyl-O-tRNA comprises at least about a 25%, 50%, 75%, 80%, 85%, 90%, 95% or 98% suppression activity in the presence of a cognate synthetase in response to a selector codon as compared to a control lacking the selector codon (and, typically, the cognate synthetase). A protein produced by this method is also a feature of the invention.

The invention also teaches variant orthogonal leucyl-tRNAs and variant orthogonal aminoacyl-tRNA synthetase species that display suppression activity, where the suppression activity is measured relative to the suppression activity of a leucyl-O-tRNA nucleotide sequence or an O-RS amino acid sequence provided by the present invention. For example, the invention teaches variant leucyl-O-tRNA species that display suppression activity that is at least 50%, 60%, 70%, 75%, 80%, 85%, 90%, 95%, 98% or 99% as effective as a leucyl-O-tRNA sequence provided by the examples herein (e.g., SEQ ID NOs: 1-7 and 12). Similarly, the invention teaches variant O-RS species that display suppression activity that is at least 50%, 60%, 70%, 75%, 80%, 85%, 90%, 95%, 98% or 99% as effective as an O-RS sequence provided by the examples herein (e.g., SEQ ID NO: 15 and 16).

In another aspect, the invention teaches variant orthogonal leucyl-tRNAs and variant orthogonal aminoacyl-tRNA synthetase species that display suppression activity that is equal to or greater than the suppression activity of a leucyl-O-tRNA nucleotide sequence or an O-RS amino acid sequence provided by the present specification. For example, the invention teaches variant leucyl-O-tRNA species that display suppression activity that is at least 100% as effective as a leucyl-O-tRNA sequence provided by the examples herein (e.g., SEQ ID NO: 1-7 and 12).

The compositions of the invention and compositions made by the methods of the invention optionally are in a cell. The leucyl O-tRNA/O-RS pairs or individual components of the invention can then be used in a host system's translation machinery, which results in a selected amino acid, e.g., unnatural amino acid, being incorporated into a protein. The International Application Number PCT/US2004/011786, filed Apr. 16, 2004, and WO 2002/085923, entitled “IN VIVO INCORPORATION OF UNNATURAL AMINO ACIDS” describe this process, and is incorporated herein by reference. For example, when an leucyl O-tRNA/O-RS pair is introduced into a host, e.g., Escherichia coli, the pair leads to the in vivo incorporation of selected amino acid, such as an unnatural amino acid, e.g., a synthetic amino acid, such as derivative of a leucine amino acid, which can be exogenously added to the growth medium, into a protein, in response to a selector codon. Optionally, the compositions of the present invention can be in an in vitro translation system, or in an in vivo system(s).

Essentially any protein (or portion thereof) that includes a selected amino acid, e.g., an unnatural amino acid, (and any corresponding coding nucleic acid, e.g., which includes one or more selector codons) can be produced using the compositions and methods herein. No attempt is made to identify the hundreds of thousands of known proteins, any of which can be modified to include one or more unnatural amino acid, e.g., by tailoring any available mutation methods to include one or more appropriate selector codon in a relevant translation system. Common sequence repositories for known proteins include GenBank EMBL, DDBJ and the NCBI. Other repositories can easily be identified by searching the internet.

Typically, the proteins are, e.g., at least 60%, at least 70%, at least 75%, at least 80%, at least 90%, at least 95%, or at least 99% or more identical to any available protein (e.g., a therapeutic protein, a diagnostic protein, an industrial enzyme, or portion thereof, and the like), and they comprise one or more selected amino acid. Examples of therapeutic, diagnostic, and other proteins that can be modified to comprise one or more selected amino acid, e.g., an unnatural amino acid, can be found, but not limited to, those in International Application Number PCT/US2004/011786, filed Apr. 16, 2004, and WO 2002/085923, entitled “IN VIVO INCORPORATION OF UNNATURAL AMINO ACIDS.”

In certain embodiments, the protein or polypeptide of interest (or portion thereof) in the methods and/or compositions of the invention is encoded by a nucleic acid. Typically, the nucleic acid comprises at least one selector codon, at least two selector codons, at least three selector codons, at least four selector codons, at least five selector codons, at least six selector codons, at least seven selector codons, at least eight selector codons, at least nine selector codons, ten or more selector codons.

Genes coding for proteins or polypeptides of interest can be mutagenized using methods well-known to one of skill in the art and described herein under “Mutagenesis and Other Molecular Biology Techniques” to include, e.g., one or more selector codon for the incorporation of a selected amino acid, e.g., an unnatural amino acid. For example, a nucleic acid for a protein of interest is mutagenized to include one or more selector codon, providing for the insertion of the one or more selected amino acids, e.g., unnatural amino acids. The invention includes any such variant, e.g., mutant, versions of any protein, e.g., including at least one selected amino acid. Similarly, the invention also includes corresponding nucleic acids, i.e., any nucleic acid with one or more selector codon that encodes one or more selected amino acid.

To make a protein that includes a selected amino acid, one can use host cells and organisms that are adapted for the in vivo incorporation of the selected amino acid via orthogonal leucyl tRNA/RS pairs. Host cells are genetically engineered (e.g., transformed, transduced or transfected) with one or more vectors that express the orthogonal leucyl tRNA, the orthogonal leucyl tRNA synthetase, and a vector that encodes the protein to be derivatized. Each of these components can be on the same vector, or each can be on a separate vector, two components can be on one vector and the third component on a second vector. The vector can be, for example, in the form of a plasmid, a bacterium, a virus, a naked polynucleotide, or a conjugated polynucleotide.

Defining Polypeptides by Immunoreactivity

Because the polypeptides of the invention provide a variety of new polypeptide sequences (e.g., comprising selected amino acids (e.g., unnatural amino acids) in the case of proteins synthesized in the translation systems herein, or, e.g., in the case of the novel synthetases, novel sequences of standard amino acids), the polypeptides also provide new structural features which can be recognized, e.g., in immunological assays. The generation of antisera, which specifically bind the polypeptides of the invention, as well as the polypeptides which are bound by such antisera, are a feature of the invention. The term “antibody,” as used herein, includes, but is not limited to a polypeptide substantially encoded by an immunoglobulin gene or immunoglobulin genes, or fragments thereof which specifically bind and recognize an analyte (antigen). Examples include polyclonal, monoclonal, chimeric, and single chain antibodies, and the like. Fragments of immunoglobulins, including Fab fragments and fragments produced by an expression library, including phage display, are also included in the term “antibody” as used herein. See, e.g., Paul, Fundamental Immunology, 4th Ed., 1999, Raven Press, New York, for antibody structure and terminology.

In order to produce antisera for use in an immunoassay, one or more of the immunogenic polypeptides is produced and purified as described herein. For example, recombinant protein can be produced in a recombinant cell. An inbred strain of mice (used in this assay because results are more reproducible due to the virtual genetic identity of the mice) is immunized with the immunogenic protein(s) in combination with a standard adjuvant, such as Freund's adjuvant, and a standard mouse immunization protocol (see, e.g., Harlow and Lane (1988) Antibodies, A Laboratory Manual, Cold Spring Harbor Publications, New York, for a standard description of antibody generation, immunoassay formats and conditions that can be used to determine specific immunoreactivity.

Additional details on proteins, antibodies, antisera, etc. can be found in, e.g., International Application Number PCT/US2004/011786, filed Apr. 16, 2004, WO 2002/085923, entitled “IN VIVO INCORPORATION OF UNNATURAL AMINO ACIDS;” International Application Numbers PCT/US2003/32870, filed Oct. 15, 2003; and PCT/US2003/41346, filed Dec. 22, 2003.

Kits

Kits are also a feature of the invention. For example, a kit for producing a protein that comprises at least one selected amino acid, e.g., an unnatural amino acid, in a cell is provided, where the kit includes a container containing a polynucleotide sequence encoding an leucyl O-tRNA, and/or an leucyl O-tRNA, and/or a polynucleotide sequence encoding an leucyl O-RS, and/or an leucyl O-RS. In one embodiment, the kit further includes at least selected amino acid. In another embodiment, the kit further comprises instructional materials for producing the protein.

### EXAMPLES

The following examples are offered to illustrate, but not to limit the claimed invention. One of skill will recognize a variety of non-critical parameters that may be altered without departing from the scope of the claimed invention.

### Example 1

### Adaptation of an Orthogonal Archaeal Leucyl-tRNA and Synthetase Pair for Four-Base, Amber, and Opal Suppression

Recently, it has been shown that an amber suppressor tRNA-aminoacyl tRNA synthetase pair derived from the tyrosyl-tRNA synthetase of Methanococcus jannaschii can be used to genetically encode unnatural amino acids in response to the amber nonsense codon, TAG. This pair is unable to decode either the opal nonsense codon, TGA, or the four-base codon, AGGA. To overcome this, a leucyl-tRNA synthetase from Methanobacterium thermoautotrophicum and leucyl tRNA derived from Halobacterium sp. NRC-1 was adapted as an orthogonal tRNA-synthetase pair in E. coli to decode amber (TAG), opal (TGA) and four-base (AGGA) codons. To improve the efficiency and selectivity of the suppressor tRNA, extensive mutagenesis was performed on the anticodon loop and acceptor stem. The two most significant criteria required for an efficient amber orthogonal suppressor tRNA are a CU(X)XXXAA anticodon loop and the lack of non-canonical or mismatched base pairs in the stem regions. These changes afford only weak suppression of TGA and AGGA. However, this information, together with an analysis of sequence similarity of multiple native archaeal tRNA sequences, led to efficient, orthogonal suppressors of opal codons and the four-base codon, AGGA. Ultimately, these additional orthogonal pairs can be used to genetically incorporate multiple unnatural amino acids into proteins.

A great deal of effort has focused on the cotranslational incorporation of unnatural amino acids into proteins. Early work demonstrated that the translational machinery of E. coli would accommodate amino acids similar in structure to the common twenty (Hortin and Boime (1983) Methods Enzymol. 96, 777-784). This work was further extended by relaxing the specificity of endogenous E. coli synthetases so that they activate unnatural amino acids as well as their cognate natural amino acid. Moreover, it was shown that mutations in editing domains could also be used to extend the substrate scope of the endogenous synthetase (Doring et al., (2001) Science 292, 501-504). However, these strategies are limited to recoding the genetic code rather than expanding the genetic code and lead to varying degrees of substitution of one of the common twenty amino acids with an unnatural amino acid.

Later it was shown that unnatural amino acids could be site-specifically incorporated into proteins in vitro by the addition of chemically aminoacylated orthogonal amber suppressor tRNAs to an in vitro transcription/translation reaction (Noren et al., (1989) Science 244, 182-188; Bain et al., (1989) J. Am. Chem. Soc. 111, 8013-8014; Dougherty (2000) Curr. Opin. Chem. Biol. 4, 645-652; Cornish et al., (1995) Angew. Chem., Int. Ed. 34, 621-633). It is clear from these studies that the ribosome and translation factors are compatible with a large number of unnatural amino acids, even those with unusual structures. Unfortunately, the chemical aminoacylation of tRNAs is difficult, and this method can only produce microgram-scale quantities of protein due to the stoichiometric nature of the process. A catalytic in vivo method could overcome these limitations, and would also permit the study of proteins containing unnatural amino acids in living cells.

In order to add additional synthetic amino acids to the genetic code in vivo it is necessary to generate a 21^{st} “orthogonal pair” of synthetase and tRNA that can function efficiently in the translational machinery. The synthetase should not cross-react with any of the endogenous tRNAs (40 in E. coli), and the orthogonal tRNA should not be aminoacylated by any of the endogenous synthetases (21 in E. coli). The tRNA should decode only a specific new codon that is not decoded by any endogenous tRNA, and the synthetase should charge its tRNA with only a specific unnatural amino acid. We have successfully generated an orthogonal tRNA-synthetase pair from tyrosyl-tRNA synthetase of Methanococcus jannaschii which satisfies these requirements. This system has been used to incorporate a series of unnatural amino acids including keto amino acids (Wang et al., (2003) Proc. Natl. Acad. Sci. U.S.A. 100, 56-61), photocrosslinking amino acids (Chin et al., (2002) Proc. Natl. Acad. Sci. U.S.A. 99, 11020-11024; Chin et al., (2002) J. Am. Chem. Soc. 124, 9026-9027), and heavy atom containing amino acids selectively into proteins in response to the TAG codon.

Several other orthogonal pairs have been reported. Glutaminyl (Liu and Schultz (1999) Proc. Natl. Acad. Sci. U.S.A. 96, 4780-4785), aspartyl (Pastrnak et al., (2000) Helv. Chim. Acta 83, 2277-2286), and tyrosyl (Ohno et al., (1998) J. Biochem. (Tokyo, Jpn.) 124, 1065-1068; Kowal et al., (2001) Proc. Natl. Acad. Sci. U.S.A. 98, 2268-2273) systems derived from S. cerevisiae tRNAs and synthetases have been described for the potential incorporation of unnatural amino acids in E. coli. Systems derived from the E. coli glutaminyl (Kowal et al., (2001) Proc. Natl. Acad. Sci. U.S.A. 98, 2268-2273) and tyrosyl (Edwards and Schimmel (1990) Mol. Cell. Biol. 10, 1633-1641) synthetase have been described for use in S. cerevisiae. The E. coli tyrosyl system can also function in mammalian cells and has been used for the incorporation of 3-iodo-L-tyrosine in vivo (Sakamoto et al., (2002) Nucleic Acids Res. 30, 4692-4699). All of these systems have made exclusive use of the amber stop codon. To expand the genetic code beyond twenty-one amino acids, other orthogonal pairs and unique codons need to be identified.

A desired property of any orthogonal pair are a codon that is unique within the genetic code and that will not cross-react with noncognate tRNAs. In addition to the amber stop codon (TAG), the opal nonsense codon (TGA) is one such candidate. A genetic code in which TAG and TGA encoded unnatural amino acids could encode 22 amino acids while preserving the ochre nonsense codon, UAA, which is the most abundant termination signal. The suppression of opal codons is robust in vivo but has not been frequently used for the incorporation of unnatural amino acids in vitro due to high background readthrough of TGA codons (Cload et al., (1996) Chem. Biol. 3, 1033-1038). Another possible codon involves unnatural base pairs. Unnatural amino acids have been incorporated in response to novel codons containing the unnatural base (iso-dC)AG (Piccirilli et al., (1990) Nature 343, 33-37) or pyridin-2-one (Hirao et al., (2002) Nat. Biotechnol. 20, 177-182) using an in vitro translation system. Adaptation of unnatural base pairs for the incorporation of unnatural amino acids into proteins in vivo, need the faithful replication and transcription of unnatural base pairs in DNA and RNA (Wu et al., (2002) J. Am. Chem. Soc. 124, 14626-14630). Another codon that can used to encode additional amino acids are four- and five-base codons. Using a library of tRNAs with randomized anticodon loops coupled with a selection scheme, several highly efficient and non cross-reactive four- and five-base codons, were identified, including AGGA, UAGA, CCCU, and UAGA (Magliery et al., (2001) J. Mol. Biol. 307, 755-769; Anderson et al., (2002) Chem. Biol. 9, 237-244).

Regardless of the codon chosen, it is useful to generate additional orthogonal tRNA-synthetase pairs that can translate these codons with high fidelity and good efficiency. Because the tRNA anticodon loop is a major identity element for recognition by most synthetases, one must identify a synthetase that does not recognize these identity elements in order to generate suppressor tRNAs for these unusual codons. The leucyl-, seryl-, and alanyl-tRNA synthetases of E. coli are well known to tolerate extensive substitutions in the anticodon loop (Shimizu et al., (1992) J. Mol. Evol. 35, 436-443; Kleina (1990) J. Mol. Biol. 213, 705-717; Sampson and Saks (1993) Nucleic Acids Res. 21, 4467-4475). Some homologous archaeal or eukaryotic synthetases may have similar properties. Herein are derivatives of a leucyl-tRNA synthetase from Methanobacterium thermoautotrophicum and leucyl tRNAs derived from Halobacterium sp. NRC-1 that act as orthogonal tRNA-synthetase pairs for the amber codon in E. coli. Moreover, information gained in these studies, together with multiple sequence alignments of native archaeal tRNA sequences, allowed us to design efficient orthogonal suppressor tRNAs of opal codons and a four-base codon, AGGA.

Material and Methods

Strains, plasmids, and materials. All in vivo manipulations were carried out in E. coli strain TOP10 (Invitrogen) in LB media at 37° C. Halobacterium sp. NRC-1 was purchased from the American Type Culture Collection (ATCC). PCR was carried out according to standard protocols with a mixture of Taq (Promega) and Pfu (Stratagene) polymerases. Oligonucleotides were synthesized by Genosys, Operon, or the UCSF Biomolecular Resource Center. For oligonucleotides containing degenerate bases, the phosphoramidites were premixed to avoid bias. Standard protocols were employed for subcloning with restriction enzymes (NEB) and T4 DNA ligase (NEB). Plasmids were introduced into E. coli by electroporation. Sequence analysis was performed using the Genetics Computer Group, Inc. (GCG) software. The sequences of all plasmids were confirmed by restriction mapping and sequencing.

Cloning of tRNA synthetase genes. Genomic DNA was either purchased from ATCC or was prepared from a cell pellet purchased from ATCC. Genomic DNA was extracted using the DNeasy kit (Qiagen). Synthetase genes were amplified from genomic DNA by PCR then subcloned into the NcoI and either EcoRI, KpnI, or PvuII sites of plasmid pKQ. More details on the cloning of these genes can be found Table 2 and is also available on the Internet at http://pubs.acs.org. Plasmid pKQ contains the ribosome binding site, multiple cloning site, and rrnB terminator from plasmid pBAD-Myc/HisA (Invitrogen) under control of a constitutive glutamine promoter. The plasmid also contains a ColE1 origin of replication, and a kanamycin resistance gene for plasmid maintenance.

Constructions of reporter plasmids. Beta-lactamase reporter plasmids were constructed from plasmid pACKO-Bla. This plasmid was constructed with a p15a origin, a chloramphenicol resistance gene, and unique sites for insertion of a gene for β-lactamase and a tRNA under control of the strong, constitutive lpp promoter. Site A184 of the β-lactamasegene was changed to TAG, AGGA, or TGA by an overlap PCR strategy, and the genes were subcloned into the AatII and XmaI sites of pACKO-Bla to give plasmids pACKO-A184TAG, pACKO-A184AGGA, and pACKO-A184TGA.

Constructions of tRNA plasmids. Genes for individual tRNAs and for tRNA libraries were constructed by extension reactions and subcloned into the EcoRI and PstI sites of pACKO-Bla derivatives. All libraries represented at least 10-fold more members than the theoretical size of the library to ensure high coverage.

Measurement of suppression efficiency. A series of LB agar plates were prepared with 25 μg/mL of kanamycin, 25 μg/mL of chloramphenicol, and concentrations of ampicillin between 5 and 1000 μg/mL. Synthetase and tRNA plasmids were cotransformed and plated at densities below 100 cells per plate. Suppression efficiency was reported as the highest concentration at which cells survived to form colonies among a series of plates for which the next highest and lowest concentrations would be within 20% of the reported value.

Selection of libraries and characterization of selectants. All tRNA libraries were subjected to ampicillin selection and the surviving colonies were isolated and sequenced by the method described previously (Magliery et al., (2001) J. Mol. Biol. 307, 755-769). Briefly, libraries were spread on LB plates containing 25 μg/mL of kanamycin and chloramphenicol for plasmid maintenance, and varying concentrations of ampicillin for selection. After 24 hours of growth, the plates were scraped, and the cells were diluted slightly then spread again on ampicillin plates. After colonies appeared, plates were again scraped and plated at dilute cell densities on a range of plates with different ampicillin concentrations. Selectants were isolated, sequenced, and then confirmed by retransformation into cells containing synthetase-expressing plasmids.

Beta-galactosidase reporter assays. The full-length lacZ gene of plasmid pBAD-Myc/His/lacZ (Invitrogen) was amplified by PCR and subcloned into plasmid pLASC to obtain plasmid pLASC-lacZ. This pSC101-derived plasmid expresses lacZ gene under the control of an lpp promoter and has an ampicillin resistance gene for plasmid maintenance. Derivatives of this plasmid were constructed wherein Leu-25 of the peptide VVLQRRDWEN of lacZ was replaced by TAG, TGA, or AGGA codons, or sense codons for tyrosine, serine, or leucine. The appropriate pLASC-lacZ-, pACKO-Bla-, and pKQ-derived plasmids were cotransformed and grown to an OD₆₀₀ of 0.5. Beta-galactosidase assays were performed in quadruplicate using the BetaFluor™ β-Galactosidase Assay Kit (Novagen). Percent suppression was calculated as the percentage of activity for a sample relative to the value observed from the pLASC-lacZ construct with the corresponding sense codon at position 25. Cells containing pLASC-lacZ plasmids with sense codons at position 25 were also assayed by 2-nitrophenyl-β-D-galactopyranoside assays (Miller (1972) Experiments in molecular genetics, Cold Spring Harbor Laboratory, Cold Spring Harbor, N.Y.), and activity was calculated in Miller units.

Purification of synthetase proteins. Synthetase genes were cloned in frame with the C-terminal myc/his tag of pBAD-Myc/HisA (Invitrogen). Protein purification was performed with the Qiaexpressionist kit (Qiagen) by the manufacturer's protocol under native conditions. Protein concentrations were measured by the BCA Protein Assay Kit (Pierce) and analyzed by SDS-PAGE.

In vitro aminoacylation assays. Aminoacylation assays were performed by methods described previously (Hoben and Soll (1985) Methods Enzymol. 113, 55-59) in 20 μL reactions containing 50 mM Tris-Cl, pH 7.5, 30 mM KCl, 20 mM MgCl₂, 3 mM glutathione, 0.1 mg/mL BSA, 10 mM ATP, 1 μM (79 Ci/mmol) [3H] leucine (Amersham), 750 nM synthetase, and 0, 2, 10, or 40 μM crude total tRNA. Crude total E. coli tRNA was purchased from Roche, and halobacterial tRNA was extracted from cultures of Halobacterium sp. NRC-1 with the RNA/DNA Extraction Kit (Qiagen).

Detailed information for the cloning of archaeal leucyl-tRNA synthetases can be found below and is also available on the Internet at http://pubs.acs.org.

TABLE 2
CLONING OF ARCHAEAL LEUCYL-tRNA SYNTHETASES INTO PLASMID pKQ

| | Accession | ATCC | Forward | Reverse | |
|---|---|---|---|---|---|
| Organism | number | Number | Oligo | Oligo | Restriction enzymes |
| Halobacterium sp. NRC-1 | NP_280869.1 | 700922 | ca214 | ca215 | NcoI/EcoRI |
| Escherichia coli (strain HB101) | P07813 | N/A | ca244 | ca215 | NcoI/EcoRI |
| Methanococcus jannaschii | Q58050 | 43067D | ca246 | ca247 | NcoI/KpnI |
| Archaeoglobus fulgidus | O30250 | 49558D | ca261 | ca247 | NcoI/KpnI |
| Aeropyrum pernix K1 | Q9YD97 | 700793D | ca263 | ca264 | BspHI/EcoRI (subcloned into |
| | | | | | NcoI/EcoRI sites) |
| Pyrococcus horikoshii | O58698 | 700860 | ca265 | ca266 | NcoI/EcoRV (subcloned into |
| | | | | | NcoI/Pvull sites) |
| Methanobacterium | O27552 | 700791 | ca274 | ca275 | BsmBI (subcloned into |
| thermoautotrophicum | | | | | NcoI/EcoRI sites) |

Oligo Sequences

| | |
|---|---|
| ca214 | GGTTTCCATGGGAGAGCAAGCCACCTAC |
| ca215 | GGTTTGGAATTCAGTCGTCGGCTTCGTCG |
| ca244 | CGAAACCATGGAAGAGCAATACCGCCCGGAAG |
| ca245 | CCAAAGAATTCCCGCCAACGACCAGATTGAGGAG |
| ca246 | CGAAACCATGGTTATGATTGACTTTAAAG |
| ca247 | CGAAAGGTACCTTGTATTCAAGATAAATAGCTGG |
| ca261 | GCGAACCATGGGCGATTTCAGGATAATTGAG |
| ca262 | CAATTGGTACCTTAAGCAACATAAATCGCG |
| ca263 | GGATTATCATGAAGCGACTAAAGGCCGTGGAGGAG |
| ca264 | CACTTGAATTCTTAGCCTCCTCTCTTCTCCGC |
| ca265 | CGAATCCATGGCTGAGCTTAACTTCAAGG |
| ca266 | GGATGGATATCACTCGATGAAGATGGCAG |
| ca274 | GGAGACGTCTCTCATGGATATTGAAAGAAAATGGCG |
| ca275 | CGTTACGTCTCGAATTGGAAAAGAGCTGTCTGAGG |

Results and Discussion

Identification of orthogonal tRNAs. Previous studies (Kwok and Wong (1980) Can. J. Biochem. 58, 213-218) have shown that halobacterial tRNAs are inefficiently charged by the E. coli leucyl-tRNA synthetase. The similarity between the halobacterial and other archaeal leucyl-tRNAs (see FIG. 1, Panel C) led us to believe that tRNAs from other archaeans can also be orthogonal to the E. coli synthetases. The sequences were chosen to broadly represent the family of archaeal leucyl-tRNAs and included tRNA₃^{Leu} of Archaeoglobus fulgidus (AfL3), tRNA₄^{Leu} of Halobacterium sp. NRC-1 (HhL4), tRNA₂^{Leu} of Methanococcus jannaschii (MjL2), tRNA₅^{Leu} of Pyrococcus furiosus (PfL5), and tRNA₂^{Leu} of Pyrococcus horikoshi (PhL2) (see FIG. 1, Panel C for sequences). In all cases, the anticodon was changed to CUA, and CCA was added to the 3′ terminus if the sequence was not present in the source gene to obtain an amber suppressor tRNA.

To measure the activity of suppressor tRNAs, a selection system was developed based on the in vivo suppression of nonsense or frameshift mutations introduced into the gene for β-lactamase (bla). Reporter genes for bla variants with TAG, AGGA, and TGA at position A184 (a permissive site (Liu and Schultz (1999) Proc. Natl. Acad. Sci. U.S.A. 96, 4780-4785)) were constructed in plasmid pACKO-Bla, a medium copy plasmid derived from pACYC184. Bacteria transformed with these reporter constructs are unable to grow on LB agar plates with ampicillin concentrations greater than 5 μg/mL, only slightly higher than the value (2 μg/mL ampicillin) observed for bacteria transformed with no plasmids. Plasmids derived from pACKO-Bla can also express tRNA genes under the control of a strong lpp promoter. When the robust amber suppressor gene supD, a tRNA efficiently charged by E. coli seryl tRNA synthetase, is expressed from pACKO-A184TAG (which encodes the A184TAG variant of bla), host bacteria survive at an ampicillin concentration of 1000 μg/mL. In contrast, in the case of an orthogonal tRNA, which cannot be efficiently charged by endogenous E. coli synthetases, ampicillin resistance should be less than 5 ug/mL. Conversely, if the tRNA is not orthogonal, or if a heterologous synthetase capable of charging the tRNA is co-expressed in the system, a higher level of ampicillin resistance should be observed.

The genes for the five potential orthogonal amber suppressor tRNAs were integrated into pACKO-A184TAG. E. coli hosts expressing the HhL4-derived suppressor, designated HL(TAG)1, could survive to only 5 μg/mL ampicillin, the MjL2- and PhL2-derived suppressors to 7 μg/mL, and the PfL5- and AfL3-derived suppressors to 20 μg/mL ampicillin. Therefore, all five suppressor tRNAs are either weak suppressor tRNAs or are inefficiently charged by E. coli aminoacyl-tRNA synthetases.

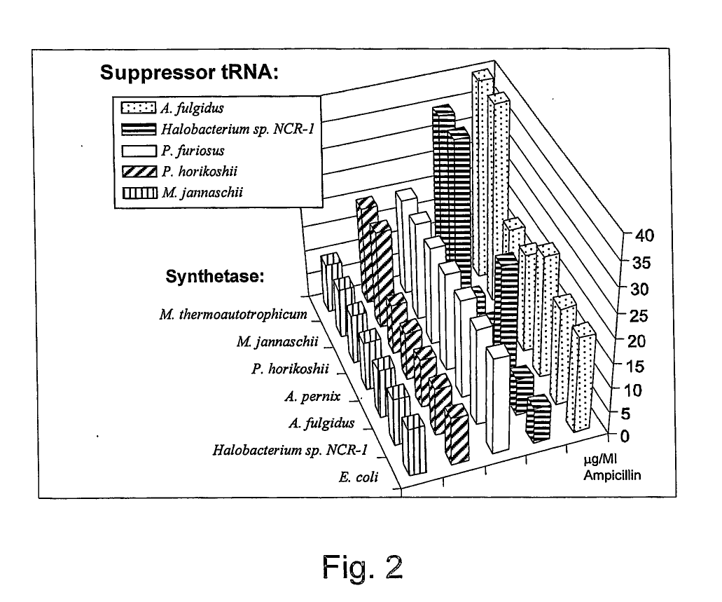

Cloning of archaeal leucyl-tRNA synthetases. Due to the high homology of the archaeal leucyl-tRNAs, we anticipated that the archeal leucyl-tRNA synthetases might have similar tRNA recognition properties. Therefore both species specific and cross species combinations of archeal leucyl-tRNAs and synthetases were examined in order to find an optimal pair for use in E. coli. The leucyl-tRNA synthetases from Archaeoglobus fulgidus (AfLRS), Aeuropyrum pernix (ApLRS), Halobacterium sp. NRC-1 (HhLRS), Methanococcus jannaschii (MjLRS), Methanobacterium thermoautotrophicum (MtLRS), and Pyrococcus horikoshi (PhLRS) were chosen as initial candidates due to the availability of the genome sequences and commercial availability of the organisms. The genes for these synthetases were cloned under the control of a constitutive glutamine promoter on the high copy plasmid, pKQ, which was constructed from pBR322 and contains a kanamycin resistance gene. The leuS gene from E. coli (EcLRS) was also cloned as a negative control. Synthetase expression plasmids and reporter constructs were cotransformed and assayed for activity by ampicillin selection (FIG. 2). In general, the reporter plasmid containing the HhL4 suppressor tRNA, HL(TAG)1, gave the largest enhancement in suppression efficiency upon cotransformation with synthetase-expressing plasmids, but the PhL2- and AfL3-derived tRNAs also show a suppression enhancement. The MjL2- and PfL5-derived suppressor tRNAs survive to the same concentrations of ampicillin regardless of whether or not the archaeal synthetase is present, and were not pursued further. From all 35 combinations of synthetase and reporter plasmids, the highest levels of ampicillin resistance result when the synthetases, MtLRS or MjLRS, are expressed with the HhL4-derived suppressor tRNA. The AfLRS construct gives slightly lower levels of resistance, and all other synthetases give no increase in suppression efficiency over background levels. With MjLRS or MtLRS, cells expressing HL(TAG)1 survive to 35 μg/mL ampicillin, but only 5 μg/mL with the E. coli synthetase or plasmid lacking the synthetase. Cells expressing AfLRS can survive to 25 μg/mL ampicillin when coexpressed with HL(TAG)1. From these in vivo suppression screens, three synthetases, MtLRS, MjLRS, and AfLRS, were identified as candidates for an orthogonal pair with the HhL4-derived amber suppressor tRNA, HL(TAG)1.

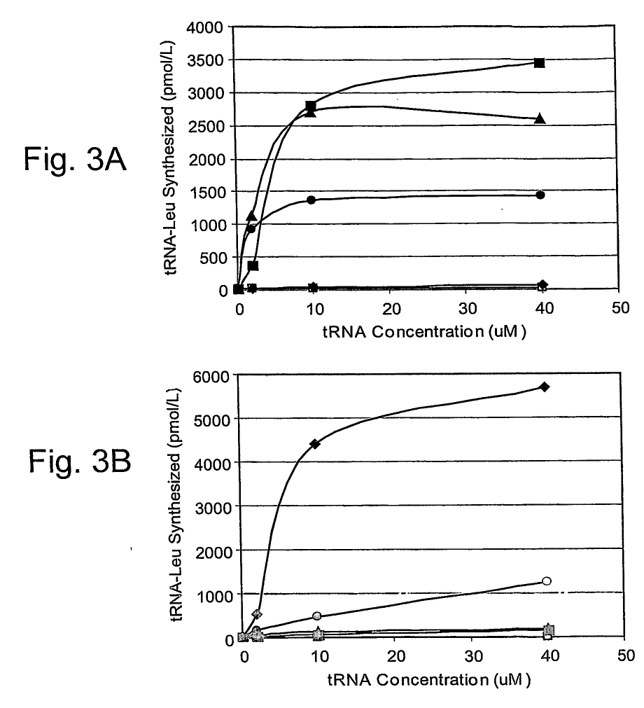

In vitro charging assays. An in vivo suppression screen can distinguish active and inactive aminoacyl-tRNA synthetases, but it cannot distinguish an orthogonal synthetase from one that cross-reacts with E. coli tRNA. To determine the permissiveness of AfLRS, MjLRS, and MtLRS for E. coli tRNA, the synthetases were overexpressed, purified, and then subjected to in vitro aminoacylation assays to measure their ability to charge E. coli tRNA. AfLRS, MjLRS, and MtLRS were purified from an arabinose promoter over-expression system by Ni-NTA affinity chromatography in yields of 14, 8, and 3 mg/L respectively. In vitro aminoacylation assays were performed with tritium-labeled leucine and either E. coli or Halobacterium NRC-1 total tRNA (FIG. 3, Panels A and B). Based on the charging of 10 μM crude total tRNA, MtLRS and AfLRS charge halobacterial tRNA 54- and 21-fold more efficiently than E. coli tRNA, respectively. The MjLRS enzyme, however, shows only a 6-fold preference for halobacterial tRNA. The E. coli enzyme was 100-fold more efficient at charging E. coli crude total tRNA than halobacterial tRNA. Therefore, MtLRS and AfLRS are good candidates for orthogonal aminoacyl-tRNA synthetases with respect to E. coli tRNA, but MjLRS is not. Since MtLRS showed a higher level of suppression with HL(TAG)1 in vivo than did AfLRS, the MtLRS/HL(TAG)1 pair was carried forward as a potential new orthogonal pair for use in E. coli.

Optimization of the tRNA anticodon loop. The robust endogenous amber suppressor supD confers survival to 1000 μg/mL ampicillin when expressed from pACKO-A184TAG. In contrast, cells expressing the MtLRS/HL(TAG)1 pair survive to only 35 μg/mL ampicillin, which corresponds to a 2.9% suppression efficiency as determined from β-galactosidase assays (Table 1). We therefore sought to improve the activity of the system. Previous experiments on frameshift, missense, and nonsense suppression revealed that A37 was a highly conserved feature in robust suppressor tRNAs (Magliery et al., (2001) J. Mol. Biol. 307, 755-769). HhL4 has a G at position 37, therefore substitution of G37 to A might be expected to improve suppression efficiency. To examine this and other possible anticodon loop mutants, a library was constructed in which the 7 positions of the anticodon loop (positions 32-38, see FIG. 4, Panel A) in HhL4 were replaced with degenerate bases and subcloned into pACKO-A184TAG. The library of tRNAs was cotransformed with pKQ-MtLRS and subjected to ampicillin selection initially at 35 μg/mL ampicillin for two rounds of selection, then plated on a series of plates with increasing ampicillin concentration in the third round of selection. At the highest concentration of ampicillin for which growth was observed (500 μg/mL), the only clone found had an anticodon loop with the sequence CUCUAAA, corresponding to a simple G37A mutation (Table 1). When cotransformed with pKQ-MtLRS, this clone could survive to 500 μg/mL ampicillin. In the absence of the synthetase it survived to only 25 μg/mL ampicillin. Under similar conditions, cells containing the wild-type M. jannaschii tyrosyl orthogonal amber suppressor tRNA survive to 350 μg/mL ampicillin in the presence of the cognate synthetase and to 60 ug/mL ampicillin without the synthetase.

TABLE 1
Suppression efficiency of mutant orthogonal tRNAs.

| | |
|---|---|
| Reporter Plasmid | Miller Units |

| | |
|---|---|
| pLASC-lacZ(Leu) | 210 ± 2 |
| pLASC-lacZ(Ser) | 200 ± 5 |
| pLASC-lacZ(Tyr) | 192 ± 7 |
| pLASC-lacZ(TAG) | 1 ± 1 |
| pLASC-lacZ(AGGA) | 2 ± 1 |
| pLASC-lacZ(TGA) | 1 ± 1 |

Percent Suppression^{a}

Suppressor

| | | | |
|---|---|---|---|
| tRNA | with pKQ | with synthetase | Sequence |

| HL(TAG)1 | 0.4 ± 0.1% | 2.9 ± 0.8% | GCGAGGGTAGCCAAGCTCGGC |
|---|---|---|---|
| | | | CAACGGCGACGGACTCTAGAT |
| | | | CCGTTCTCGTAGGAGTTCGAG |
| | | | GGTTCGAATCCCTTCCCTCGC |
| | | | ACCA |
| HL(TAG)2 | 0.3 ± 0.1% | 9.6 ± 0.4% | GCGAGGGTAGCCAAGCTCGGC |
| | | | CAACGGCGACGGACTCTAAAT |
| | | | CCGTTCTCGTAGGAGTTCGAG |
| | | | GGTTCGAATCCCTTCCCTCGC |
| | | | ACCA |
| HL(TAG)3 | 1.5 ± 1.2% | 33.2 ± 4.4% | CCCAGGGTAGCCAAGCTCGGC |
| | | | CAACGGCGACGGACTCTAAAT |
| | | | CCGTTCTCGTAGGAGTTCGAG |
| | | | GGTTCGAATCCCTTCCCTGGG |
| | | | ACCA |
| HL(AGGA)1 | 0.4 ± 0.1% | 4.6 ± 2.1% | GCGAGGGTAGCCAAGCTCGGC |
| | | | CAACGGCGACGGACTTCCTAA |
| | | | TCCGTTCTCGTAGGAGTTCGA |
| | | | GGGTTCGAATCCCTTCCCTCG |
| | | | CACCA |
| HL(AGGA)2 | 0.7 ± 0.3% | 14.9 ± 6.1% | GCGAGGGTAGCCAAGCTCGGC |
| | | | CAACGGCGACGGACTTCCTAA |
| | | | TCCGTTCTCGTAGGAGTTCGA |
| | | | GGGTTCGAATCCCTCCCCTCG |
| | | | CACCA |
| HL(AGGA)3 | 7.4 ± 0.4% | 35.5 ± 1.4% | GCGGGGGTTGCCGAGCCTGGC |
| | | | CAAAGGCGCCGGACTTCCTAA |
| | | | TCCGGTCCCGTAGGGGTTCCG |
| | | | GGGTTCAAATCCCCGCCCCCG |
| | | | CACCA |
| HL(TGA)1 | 4.7 ± 1.5% | 60.8 ± 7.0% | GCGGGGGTTGCCGAGCCTGGC |
| | | | CAAAGGCGCCGGACTTCAAAT |
| | | | CCGGTCCCGTAGGGGTTCCGG |
| | | | GGTTCAAATCCCCGCCCCCGC |
| | | | ACCA |
| J17^{b} | 0.2 ± 0.1% | 18.5 ± 4.8% | CCGGCGGTAGTTCAGCAGGGC |
| | | | AGAACGGCGGACTCTAAATCC |
| | | | GCATGGCGCTGGTTCAAATCC |
| | | | GGCCCGCCGGACCA |
| SupD | 42.8 ± 7.1% | ND | GGAGAGATGCCGGAGCGGCTG |
| | | | AACGGACCGGTCTCTAAAACC |
| | | | GGAGTAGGGGCAACTCTACCG |
| | | | GGGGTTCAAATCCCCCTCTCT |
| | | | CCGCCA |
| Ser2AGGA | 25.2 ± 0.1% | ND | GGAGAGATGCCGGAGCGGCTG |
| | | | AACGGACCGGTCTTCCTAAAC |
| | | | CGGAGTAGGGGCAACTCTACC |
| | | | GGGGGTTCAAATCCCCCTCTC |
| | | | TCCGCCA |
| ^{a}β-Galactosidase activity was determined for tRNA reporter plasmids derived from pACKO-Bla cotransformed with the appropriate pLASC-lacZ mutant and either a synthetase-expressing plasmid or a plasmid with no synthetase. Activity is reported as the percentage of activity observed relative to the value observed from the pLASC-lacZ construct with a leucyl (wild-type), seryl, or tyrosyl sense codon at position 25. In each case, the codon at position 25 of lacZ is | | | |
| # designated in parentheses. | | | |

^{b}J17, the M. jannaschii tyrosyl amber suppressor tRNA with improved orthogonality (Wang and Schultz (2001) Chem. Biol. 8, 883-890) was expressed in plasmid pACKO-A184TAG in the presence of pLASC-lacZ(TAG) and either pKQ or pBK-JYRS.

Randomization of leucyl acceptor stem. Although the activity of the HhL4-derived amber tRNA was significantly improved with the G37A mutation, the suppression level in the absence of the synthetase increased from 5 to 25 μg/mL ampicillin. To overcome the undesired increase in background suppression, a mutant of the HhL4-derived tRNA was sought that would not cross react with E. coli aminoacyl-tRNA synthetases. Almost all of E. coli synthetases recognize bases within the acceptor stem of their cognate tRNAs. Therefore, we anticipated that mutations within this region of the tRNA might eliminate interactions between the orthogonal tRNA and the cross-reactive synthetase. A library in which the 3 terminal base pairs of the acceptor stem and the discriminator base were randomized (positions 1-3 and 70-73, the randomized region is outlined in FIG. 4, Panel A) was constructed from the HL(TAG)₂ mutant tRNA and subcloned into pACKO-A184TAG.

To identify members of this tRNA library that retained activity but were even poorer substrates for endogenous synthetases, a selection strategy was adopted from previous work on the M. jannaschii system (Wang and Schultz (2001) Chem. Biol. 8, 883-890). To isolate a pool of mutant tRNAs that had comparable activity to the G37A mutant of the HhL4-derived tRNA, the tRNA library in which the acceptor stem was randomized was cotransformed with pKQ-MtLRS and subjected to two rounds of positive selection at 500 μg/mL ampicillin. Six clones surviving the positive selection were sequenced, and all were unique and conserved the discriminator base, A73 (FIG. 4, Panels A and B). In all cases the stem positions had standard Watson-Crick base pairs. To identify members of the pool of active clones that would not be charged by endogenous aminoacyl-tRNA synthetases, the surviving tRNA-expressing plasmids were transferred into cells containing a barnase reporter plasmid, pSCB2. This plasmid contains the gene for the RNase, barnase, with two TAG codons at permissive positions 2 and 44, under control of the arabinose promoter, as well as the gene for β-lactamase. Any tRNA that is aminoacylated by an endogenous E. coli synthetase will result in suppression of the nonsense codons and cell death. The cells were plated on LB plates containing 25 μg/mL of chloramphenicol, 50 μg/mL of ampicillin to maintain the plasmids, and 0.2% arabinose to induce expression of the barnase gene. Sixteen survivors were sequenced, and three unique sequences were identified. All three clones had reversed the 3:70 base pair from G:C to C:G. Of these, mutant HL(TAG)₃ gave the highest level of suppression in the presence of MtLRS (600 μg/mL ampicillin) and only survived to 7.5 μg/mL ampicillin without the synthetase. These values correspond to 33.2% suppression in the presence of MtLRS and 1.5% in the absence of the synthetase as determined by β-galactosidase amber suppression assays (see Table 1). By comparison, the mutant M. jannaschii suppressor tRNA, J17, gives values of 18.5% and 0.2% with and without the M. jannaschii tyrosine synthetase, respectively.

Identification of AGGA suppressors. To expand the list of codons that can be used for unnatural amino acid mutagenesis, a tRNA that could efficiently suppress a four-base codon was sought. Previous studies indicated that the four-base codon AGGA can be efficiently suppressed in E. coli, and tRNAs with 8 nucleotide anticodon loops were the most efficient suppressors of AGGA codons (Magliery et al., (2001) J. Mol. Biol. 307, 755-769). A β-lactamase reporter plasmid analogous to the TAG reporter was constructed but with A184 replaced by AGGA instead of TAG. Normal translation in the absence of a +1 frameshift suppressor tRNA should result in missense errors downstream of position 184 and premature truncation of the protein. A library of tRNAs derived from the HhL4 tRNA was constructed in which the 7 nucleotide anticodon loop was replaced with 8 random nucleotides. The library was subcloned into pACKO-A184AGGA, cotransformed with pKQ-MtLRS, and then subjected to ampicillin selection. At the highest concentration of ampicillin at which growth was observed, 75 μg/mL, only one clone, HL(AGGA)1, was found. This clone had the anticodon loop sequence CUUCCUAA. As was the case with the bla A184TAG reporter plasmid, cells transformed with pACKO-A184AGGA can survive to only 5 μg/mL ampicillin in the absence of a suppressor tRNA. Therefore, the clone identified, HL(AGGA)1, is a weak suppressor of AGGA codons.

During these experiments, serendipitous mutants capable of surviving up to 300 μg/mL ampicillin were identified. These mutants were no longer orthogonal, and all had multiple point mutations relative to the parent sequence. All of the clones contained the substitution T65C. This mutation corrects the G:U mismatch present in the TψC loop stem, suggesting that this G:U base pair might be detrimental to suppressor activity. We therefore decided to randomize this base; a library was made in which the 49:65 base pair was randomized in HL(AGGA)1. The library was subcloned into pACKO-A184AGGA, and then cotransformed with pKQ-MtLRS. Of the 16 library members, the most efficient suppressor, HL(AGGA)₂, was identified by ampicillin selection. This clone contained a T65C mutation and could survive to 125 μg/mL ampicillin. Nevertheless, this level of activity was far lower than that observed for the corresponding amber suppressors. Consequently, alternative strategies were considered.

Mutations in the D-loop have been previously implicated in frameshift suppression (Tuohy et al., (1992) J. Mol. Biol. 228, 1042-1054), and we next hypothesized that such mutations might improve the suppression efficiency of the AGGA suppressor. Libraries wherein the 13 nucleotides of the D-loop (position 14-21, see FIG. 4, Panel A) were replaced with 11 or 13 random nucleotides were prepared in pACKO-A184AGGA. Although a great deal of sequence diversity was observed among the survivors at the highest concentrations of ampicillin (125 μg/mL), no mutants were observed with increased activity relative to the parent tRNA.

A consensus-derived AGGA suppressor tRNA. In examining the sequence of the HhL4-derived tRNA, there was no obvious explanation for the poor activity of this suppressor. Rather than mutate HL(AGGA)2 further, we pursued an alternative approach. The archaeal leucyl-tRNAs are highly similar, varying from each other usually by only a few base substitutions (FIG. 1, Panel C). The entire family would be well represented by a library derived from a consensus sequence with many random mutations throughout. The consensus sequence was compiled with the GCG program pileup, and those positions considered degenerate by the program were changed to the most frequent base at those positions. The anticodon loop was changed to CUUCCUAA since this sequence was already shown to be the optimal sequence for an AGGA suppressor derived from HhL4. The final sequence used as the consensus sequence is shown in FIG. 5. A library was synthesized by overlap extension of oligonucleotides in which each site of the tRNA gene was synthesized as a doped mixture of 90% the consensus sequence and 10% a mixture of the other 3 bases. The library was subcloned into pACKO-A184AGGA. Sequencing of 24 naïve clones revealed that the average number of mutations per clone was 5.9, and these mutations were randomly distributed throughout the tRNA sequence. After cotransformation with pKQ-MtLRS and selection on ampicillin plates, several clones survived to 300 μg/mL of ampicillin and were found to be the original sequence with the 27:42 and 49:65 base pairs changed to the canonical base pairs T27:A42, G27:C42, or C27:G42, and G47:C65 or C47:G65 (FIG. 5). The most efficient suppressor, designated HL(AGGA)3, can survive to 300 μg/mL ampicillin in the presence of pKQ-MtLRS but to only 30 μg/mL in the absence of the synthetase, which correspond to 35.5% and 7.4% suppression, respectively, as determined by β-galactosidase assays (Table 1).

Identification of opal suppressor tRNAs. To further expand the list of codons, we sought opal suppressors derived from HhL4. A reporter plasmid, pACKO-A184TGA, was constructed in which the A184 position of β-lactamase was changed to TGA. This bla A184TGA reporter plasmid can survive to 10 μg/mL ampicillin without any suppressor tRNA present, whereas the TAG and AGGA reporters could survive to only 5 μg/mL. In the case of opal suppression, there is background read-through that leads to the production of a small amount of protein even in the absence of a suppression system. Nevertheless, this level is quite small. To identify suppressors, a library in which the anticodon loop (positions 31-38) of HhL4 was replaced with 7 degenerate nucleotides was prepared in pACKO-A184TGA. When cotransformed with pKQ-MtLRS, no members of this library could survive on ampicillin plates at 50 μg/mL. Instead of HhL4, a library was prepared in which the 8 nucleotide anticodon loop was randomized with 7 nucleotides in HL(AGGA)3, the most robust AGGA suppressor identified from the consensus sequence. At the highest concentrations of ampicillin at which growth was observed (300 μg/mL) only one clone, designated HL(TGA)1, with the sequence CUUCAAA was found. The clone can survive to 350 μg/mL ampicillin when coexpressed with pKQ-MtLRS, but can survive to only 30 μg/mL without the synthetase plasmid, which corresponds to 60.8% suppression as determined by β-galactosidase assays (Table 1). Apparently, the beneficial effects of using the consensus sequence are not limited to frameshift suppression.

Identification of new orthogonal pairs. One approach to construct orthogonal tRNA-synthetase pairs is to adapt eukaryotic or archaeal synthetases and tRNAs for use in E. coli. Several yeast synthetases, notably glutamine, aspartic acid, arginine, and tyrosine, have been shown not to recognize E. coli tRNA, and might therefore be useful for the construction of orthogonal tRNA-synthetase pairs. Unfortunately, many eukaryotic synthetases express poorly or have low specific activity in E. coli. Eukaryotic synthetases, particularly the mammalian enzymes, are often organized into large complexes (Mirande et al., (1982) EMBO J. 1, 733-736), and the low activity often observed may be related to the inability to form these complexes in E. coli.

The success of the M. jannaschii tyrosyl orthogonal pair (Wang et al., (2001) Science 292, 498-500) suggested that archaebacteria may in general be a good source of orthogonal pairs. Early work on the halophile Halobacterium cutirebrum (Kwok and Wong (1980) Can. J. Biochem. 58, 213-218) indicated that almost all the tRNAs of this archaean (notably leucine, arginine, tyrosine, serine, histidine, and proline) cannot be charged by E. coli aminoacyl-tRNA synthetases. Indeed, archaeal tRNA synthetases are more similar to their eukaryotic than prokaryotic counterparts in terms of homology and tRNA recognition elements. Unlike their eukaryotic counterparts, however, there is currently no evidence for their higher order assembly into structured multimers (Tumbula et al., (1999) Genetics 152, 1269-1276; Woese et al., (2000) Microbiol. Mol. Biol. Rev. 54,202-236). Moreover, since most archaea are thermophiles, active synthetases from archaea can be expressed in good yields in E. coli and can be readily purified in active form. Due to extensive sequencing efforts, at least 16 archaeal genome sequences are currently available, which together with the lack of introns in the genome, greatly facilitates the PCR amplification of the archaeal synthetase genes. For all of the above reasons, our attention has focused on the archaea as a source for orthogonal pairs.

Another design issue in the construction of orthogonal tRNA-synthetase pairs is the ability of the aminoacyl-tRNA synthetase to recognize mutants of the cognate tRNA with altered anticodon loops (i.e., nonsense or missense suppressors). Aminoacyl-tRNA synthetases frequently use the anticodon loop as a major positive identity element, and mutations in this region of the tRNA frequently result in impaired synthetase recognition. The leucyl-tRNA synthetases frequently lack strong anticodon recognition elements, and a leucyl orthogonal tRNA-synthetase pair can therefore be able to decode a variety of codons, including amber, opal and four-base codons. Of the archaeal leucyl-tRNA synthetases, only the enzyme from Haloferax volcanii has been thoroughly investigated (Soma et al., (1999) J. Mol. Biol. 293, 1029-1038). The synthetase does not recognize bases in the anticodon loop; instead, a highly conserved pattern of mismatches within the variable loop is the primary recognition element for the synthetase. Although the cloning of the gene for this enzyme has not been reported, the sequenced genome of a closely related archaean, Halobacterium sp. NRC-1, is available. A multiple sequence alignment of leucyl-tRNA synthetases from many phyla including archaeal, prokaryotic, and eukaryotic sequences (FIG. 1, Panel A) shows that the halophilic enzyme is unusual among the family of archaeal synthetases, having greater homology to the prokaryotic branch than the eukaryotic or archaeal branches. Unlike the synthetases, all archaeal leucyl tRNAs are highly homologous and share absolutely conserved features such as A73, G37, and a 12 nucleotide variable loop with 2 unpaired bases (FIG. 1, Panel B). The conservation of these positive recognition elements led us to believe that tRNA recognition by the other archaeal leucyl-tRNAs would be similar to recognition by the halobacterial synthetase. Consequently, these synthetases can be useful in the construction of orthogonal tRNA-synthetase pairs when combined with suppressor tRNAs derived from archaeal leucyl-tRNAs.

Because archaeal leucyl-tRNAs and synthetases are highly homologous to one another, both species-specific and cross-species combinations could potentially function as efficient orthogonal tRNA-synthetase pairs. Therefore, each of the five potential orthogonal tRNAs (AfL3, HhL4, MjL2, PfL5, and PhL2) was examined in the presence of each of the six separate archaeal synthetases (AfLRS, ApLRS, HhLRS, MjLRS, MtLRS, and PhLRS) for the ability to suppress A184TAG in bla. All five orthogonal tRNAs afforded a higher level of amber suppression in the absence of an archaeal synthetase than is observed when no amber suppressor tRNA is present in the cell. All five suppressors are, therefore, expressed, processed, and functionally charged to some degree by an endogenous E. coli synthetase. Nevertheless, only three of the five tRNAs (PhL2, AfL3, and HhL4) gave a higher level of suppression when a foreign synthetase (either MjLRS, MtLRS, or AfLRS) was coexpressed with the tRNA than was observed with no synthetase. The MjL2 and PfL5 suppressors fail to give an enhancement in suppression when coexpressed with a cognate or noncognate archaeal synthetase. Without being limited to one theory, these tRNAs may be expressed as functional suppressor tRNAs in E. coli but are unable to be charged due to incompatibility with both cognate and noncognate synthetases. In the case of MjL2, the suppressor is derived from the natural substrate for MjLRS, so it seems unlikely that the tRNA would not be charged, when other tRNAs are efficiently charged by MjLRS. Another explanation might be that the tRNAs are efficiently charged but are incompatible with the E. coli translational machinery, but this is not consistent with the fact that some suppression is observed when no archaeal synthetase is present. Another possibility is that MjL2 and PfL5 are efficiently charged with leucine, but are deacylated in an editing process by an endogenous E. coli synthetase. In any case, not all archaeal leucyl isoacceptors are equivalent in their ability to function as orthogonal amber suppressors in E. coli.

Only three of the six leucyl-tRNA synthetases (MjLRS, MtLRS, and AfLRS) cloned from archaea gave a higher level of suppression when combined with any of the five orthogonal tRNAs. In the case of HhLRS, the synthetase does not yield protein when over-expressed. Without being limited to one theory, it is most likely, PhLRS and ApLRS do not express functional protein in E. coli either, but it is also possible that the proteins are not active at 37° C., or do not recognize any of the orthogonal tRNAs tested. There was no evidence that some tRNAs are preferred substrates for a specific synthetase. Indeed, although a tRNA from M. jannaschii was one of the five orthogonal tRNAs examined, the halobacterium-derived suppressor was the preferred substrate for MjLRS. All three functional tRNAs gave the highest level of suppression when charged by MtLRS or MjLRS, and to a lesser degree with AfLRS.

Although on the whole the archaeal leucyl synthetases have similar tRNA recognition properties, it is clear from in vitro charging experiments that there are some differences in their recognition of tRNA. The charging of crude total E. coli tRNA by AfLRS and MtLRS is only 5- and 13-fold higher, respectively, than the background reaction observed with no synthetase, whereas MjLRS is able to charge E. coli tRNA 50-fold over background. Such differences in tRNA recognition among highly homologous synthetases was unanticipated, but not without precedent (Kwok and Wong (1980) Can. J. Biochem. 58, 213-218). Since aminoacyl-tRNA synthetases have evolved only to be orthogonal to the non-cognate tRNAs present in their own host's cytoplasm, it is perhaps not surprising that subtle variations in sequence or chemical modification can lead to mischarging in foreign systems.

Improving the activity of orthogonal suppressor tRNAs. To date, we have identified and characterized three orthogonal tRNA-synthetase pairs: the yeast glutamine (Liu and Schultz (1999) Proc. Natl. Acad. Sci. U.S.A. 96, 4780-4785), yeast aspartate (Pastrnak et al., (2000) Helv. Chim. Acta 83, 2277-2286), and archaeal tyrosine pairs (Wang et al., (2000) J. Am. Chem. Soc. 122, 5010-5011). Of these systems, only the tyrosine system gives levels of amber suppression comparable to the levels observed for strong native amber suppressors such as supD or supF. When expressed with a the high-copy β-lactamase reporter pBLAM (the reporter plasmid for this study was a medium-copy plasmid) in the presence of their cognate synthetase, cells containing the original glutamine, aspartate, and tyrosine orthogonal amber suppressor tRNAs can survive to 140, 60, and 1220 ug/mL ampicillin, respectively (Pastrnak et al., (2000) Helv. Chim. Acta 83, 2277-2286; Wang et al., (2000) J. Am. Chem. Soc. 122, 5010-5011). A high level of suppression may be critical to the successful modification of the amino acid specificity of synthetases using a double-sieve selection strategy (Liu and Schultz (1999) Proc. Natl. Acad. Sci. U.S.A. 96, 4780-4785). For suppression systems with low activity, it is often difficult to distinguish active and inactive synthetases in selection experiments due to their similarity in phenotype. A high level of suppression is required for the production of protein containing unnatural amino acids. Therefore, a great deal of attention has been paid to those features of orthogonal tRNAs that give rise to robust suppression.

Previous work on frameshift and amber suppression in E. coli clearly indicates that positions 31, 32, 37, and 38 of the tRNA anticodon loop have profound effects on suppression efficiency (Yarus et al., (1986) J. Mol. Biol. 192, 235-255; Smith et al., (1987) Nucleic Acids Res. 15, 4669-4686; Raftery and Yarus (1987) EMBO J. 6, 1499-1506; Kleina (1990) J. Mol. Biol. 213, 705-717). The presence of G37 in all the archaeal leucyl tRNAs led us to believe that a substitution at this position might lead to a higher suppression efficiency. Indeed, randomization of the anticodon loop showed that the most efficient suppressors have the anticodon loop CUCUAAA. Although the tRNA was toxic, the G37A mutant also emerged through selection with the M. jannaschii tyrosine system (Wang and Schultz (2001) Chem. Biol. 8, 883-890) as the most potent suppressor thus far observed for this system. Similar selection experiments with the yeast-derived glutamine and aspartate orthogonal pairs have been performed in which libraries of positions 32-38 of the anticodon loop are replaced with degenerate bases then subjected to positive ampicillin selection in the presence of the cognate synthetase (J. C. A., P. G. S., and Miro Pastrnak, unpublished results). In both cases, the anticodon loop sequence CUCUAAA afforded the highest suppression efficiency corresponding to six-fold and five-fold enhancements in the concentration of ampicillin at which growth is observed for the glutamine and aspartate systems, respectively. In at least three other systems, tRNAs with the anticodon loop sequence CUCUAAA have emerged as the most efficient amber suppressors. The anticodon loop sequence CUUCCUAA was found to be the most efficient sequence for a leucyl AGGA suppressor. Selection experiments on tRNAs with randomized anticodon loops in E. coli tRNA₂^{Ser} similarly converged on the anticodon loop sequence CUUCCUAA for AGGA suppression (Magliery et al., (2001) J. Mol. Biol. 307, 755-769), and the sequence CUUCAAA also emerged as the most efficient anticodon loop sequence for a leucyl opal suppressor. These results suggest that the preferred anticodon loop sequence is determined by interactions with endogenous translational machinery rather than the particular preferences of the aminoacyl-tRNA synthetases. Indeed, the anticodon loop may require sequence-specific modifications in order to function optimally (Soderberg and Poulter (2000) Biochemistry 39, 6546-6553; Sussman and Kim (1976) Science 192, 853-858). Alternatively, Yarus (Yarus (1982) Science 218, 646-652) has suggested that the entire anticodon stem and loop (positions 27-43 of the tRNA) together function as an “extended anticodon” that interacts with ribosome as a module. The entire sequence of this region can help to define the identity of the anticodon for proper decoding.

All three codons examined in this study were most efficiently suppressed by tRNAs with the sequence CU(X)XXXAA in the anticodon loop. Although this consensus sequence is preferred for TAG, TGA, and AGGA codons, other sequences may be preferable for other four- and five-base codons. In previous studies (Magliery et al., (2001) J. Mol. Biol. 307, 755-769), the most efficient suppressor tRNAs had bases at positions 32, 33, 37, and 38 which differed from the consensus sequence. For example, the most efficient suppressors of the codon CUAG had an anticodon loop with the sequence CGCTAGGA, deviating at both U33 and A37. In addition, some synthetases employ position 37 as a strong positive determinant for recognition, in which case a CU(X)XXXAA anticodon loop sequence can prove to be non-optimal.

Optimization of the anticodon loop sequence as described above was sufficient to provide an efficient amber suppressor tRNA for the leucine system. Optimization of the anticodon loop of the AGGA frameshift suppressors derived from HhL4 also afforded a viable tRNA. However the suppression efficiency (4.6%) of this tRNA, HL(AGGA)1, is far lower than that measured for the suppression of amber codons by HL(TAG)₂. Indeed, this suppressor permitted survival at only 75 μg/mL ampicillin, significantly less than the seryl AGGA suppressor (Ser2AGGA) identified previously (Magliery et al., (2001) J. Mol. Biol. 307, 755-769), which can survive to 275 ug/mL ampicillin when expressed in plasmid pACKO-A184AGGA. In general, the best AGGA suppressors are less active than the best amber suppressors (Anderson et al., (2002) Chem. Biol. 9, 237-244), but there appears to be something particular to HhL4 that hinders its ability to act as a frameshift suppressor. The only feature obviously different from robust four-base suppressors previously identified (Atkins et al., (1991) Annu. Rev. Genet. 25, 201-228) is the presence of a very large D loop in HhL4. Most suppressors have 9 nucleotides in the D loop and 4 base pairs in the stem. HhL4 has only 3 base pairs in the stem and 13 bases in the loop. Moreover, previous studies have shown the D loop to play a role in frameshift suppression (Tuohy et al., (1992) J. Mol. Biol. 228, 1042-1054). Not only did we see no increase in activity upon randomization of the D loop, there was also a great deal of sequence variation among the most active suppressors.

The serendipitous appearance of mutations in the G49:U65 base pair of the four-base suppressor tRNAs suggested that non-canonical base pairing in the stem regions of tRNAs has a deleterious effect on suppression efficiency. This hypothesis was further supported by a randomization and selection experiment on the acceptor stem of the HhL4-derived amber suppressor. The three terminal base pairs of the acceptor stem were simultaneously randomized. This library of tRNAs would therefore contain all combinations of mismatched and Watson-Crick base pairs. In fact, 98.4% of the theoretical members of this library should have at least one mismatched base pair. Nevertheless, in the 9 active acceptor stem mutants outlined in FIG. 4, Panel B, all positions are occupied by Watson-Crick base pairs. Similarly, the D, TψC, anticodon, and acceptor stems of the yeast glutamine amber suppressor tRNA have been individually randomized and subjected to positive selection (J. C. A. and P. G. S., unpublished results). In all surviving clones, every position in these stem regions was occupied by a Watson-Crick pair. In the parent tRNA, the 6:67 base pair is U:G. Mutation of this base pair to U:A results in a doubling of the concentration of ampicillin at which cells can grow. Also, when subjected to positive selection, the only mutations that emerged from random mutagenesis of the leucyl consensus-derived frameshift suppressor appeared at mispaired sites. Others have also noted that mispairing in stem regions adversely affects suppression efficiency (Buttcher et al., (1994) Biochem. Biophys. Res. Commun. 200, 370-377; Hou et al., (1992) Biochemistry 31, 41574160). Without being limited to one theory, it may be that tRNAs with mispaired bases are not readily folded into the correct cloverleaf structure and therefore are not readily processed and modified (Furdon et al., (1983) Nucleic Acids Res. 11, 1491-1505). A quantitative analysis of the ratio of charged to uncharged species and of the ratio of fully processed to unprocessed tRNA present in the cell could enhance our understanding of the mechanisms by which these poorly-suppressing tRNAs are impaired.

An analysis of multiple sequence alignments of many families of tRNAs reveal multiple examples of conserved non-Watson-Crick pairings. For example, a G3:U70 base pair is a conserved positive determinant for recognition by E. coli alanyl-tRNA synthetase (Martinis and Schimmel (1995) in tRNA: Structure, Biosynthesis, and Function (Soll, D., and RajBhandary, U., Eds.) pp 349-370, ASM Press, Washington, D.C.). If the element is a conserved positive determinant for recognition, then it may prove difficult to construct robust suppressor tRNAs for the cognate synthetase. Most often, however, the mispairing present in native sequences is only found in specific isoacceptors. Without being limited to one theory, these and other variations from the consensus sequence of the family of tRNAs present in individual isoacceptors may be present as a result of subtle, species-specific adaptations in positive or negative synthetase recognition, optimal processing and modification, or interactions with elongation factors. Alternatively, these variations may simply be the result of neutral evolutionary drift.

When transferred to another species, these variations are unlikely to offer to the new host's translational machinery any advantages they conferred to the source organism. Furthermore, for cross-species pairs, the synthetase is unlikely to recognize any species-specific identity elements present in the tRNA. Only those recognition elements common to the entire family are likely to be useful. Similarly, any processing or modification adaptations particular to a specific tRNA would be lost to the E. coli translational apparatus. These variations may even be deleterious to suppression efficiency, particularly when these variations are mismatched bases in stem regions. Suppressors tRNAs derived from the consensus sequence preserve only those features that are broadly shared by the entire family, and eliminate potentially deleterious variations. Therefore, suppressor tRNAs derived from the consensus sequence may in general lead to higher suppression efficiencies.

Although optimization of the anticodon loop and elimination of mispairing gave modest to large increases in suppression efficiency, these modifications were not sufficient to provide robust AGGA and opal suppressor tRNAs. Only the consensus-derived suppressors had activities comparable to the tRNAs₂^{Ser}-derived suppressors described previously (Anderson et al., (2002) Chem. Biol. 9, 237-244). A comparison of the consensus-derived sequences for HL(AGGA)3 and HL(AGGA)2 reveal that there are 14 base substitutions, but neither sequence has mispairs. Without being limited to one theory, perhaps by using the consensus sequence of the entire family of tRNAs, those bases that are specific to any particular tRNA and may be detrimental to activity are identified and eliminated.

Improving the selectivity of orthogonal suppressor tRNAs. Unfortunately, improvements in the activity of these suppressor tRNAs also brought about an undesirable increase in the level of suppression observed in the absence of synthetase. The original M. jannaschii tyrosine orthogonal suppressor tRNA was partially charged by an E. coli synthetase, but the reaction was eliminated by mutagenesis (Wang and Schultz (2001) Chem. Biol. 8, 883-890). A double sieve selection was able to identify mutants of the wild-type tRNA with excellent orthogonality, but there was also a significant loss of overall activity. Ideally, mutations could be introduced into the tRNA that would eliminate the cross-reactivity with E. coli synthetases but preserve high levels of suppression efficiency. Since aminoacyl-tRNA synthetases frequently recognize positions within the acceptor stem and discriminator base of tRNAs (Martinis and Schimmel (1995) in tRNA: Structure, Biosynthesis, and Function (Soll and RajBhandary, Eds.) pp 349-370, ASM Press, Washington, D.C.), it is likely that an E. coli synthetase that charges the orthogonal tRNA would have a positive recognition element in this region. If this determinant could be changed without destroying recognition by the foreign synthetase, activity could be preserved while eliminating the background reaction. When this strategy was applied to the HhL4-derived amber suppressor, such mutants were indeed found. Several mutants preserved or even improved suppression efficiency when coexpressed with MtLRS but had nearly background levels of amber suppression (7.5 versus 5 ug/mL ampicillin) in the absence of the synthetase. These mutants had reversed the third base pair from G:C to C:G, and an inspection of the recognition elements known for various E. coli synthetases suggests the identity of the E. coli synthetase that had cross-reacted with HhL4-derived suppressor. Both GlnRS and LysRS of E. coli conserve G3:C70 and frequently cross-react with amber suppressor tRNAs (Kleina (1990) J. Mol. Biol. 213, 705-717). Because LysRS also conserves A73, this is the more likely candidate for the cross-reactive E. coli synthetase (Freist and Gauss (1995) Biol. Chem. Hoppe-Seyler 376, 451-472; McClain et al., (1988) Science 242, 1681-1684). This strategy can be a general solution to the problem of improving the specificity of cross-reactive orthogonal tRNAs since most E. coli aminoacyl-tRNA synthetases contain positive determinants within the acceptor stem.

We have shown that the leucyl-tRNA synthetase from the archaean Methanobacterium thermoautotrophicum and mutants of a halobacterial tRNA function as an orthogonal pair in E. coli. Mutagenesis experiments showed that the two most significant criteria that lead to efficient orthogonal amber suppressor tRNAs are a CU(X)XXXAA anticodon loop and the lack of non-canonical or mismatched base pairs in the stem regions. From these selections, we have identified efficient amber, four-base, and opal orthogonal suppressor tRNAs. We have also devised a consensus strategy to rationally design efficient orthogonal tRNAs. This leucyl-orthogonal pair can be combined with the M. jannaschii pair to site-specifically incorporate two unique unnatural amino acids simultaneously into proteins in vivo.

Abbreviations and Textual Footnotes: Af, Ap, Hh, Mj, Mt, Pf, Ph, and Ec: Archaeoglobus fulgidus, Aeuropyrum pernix, Halobacterium sp. NRC-1, Methanococcus jannaschii, Methanobacterium thermoautotrophicum, Pyrococcus furiosus, Pyrococcus horikoshi, and Escherichia coli, respectively; LRS, leucyl-tRNA synthetase; bla, gene for β-lactamase; lacZ, gene for β-galactosidase.

### Example 2

### Exemplary Leucyl O-RSs and Leucyl O-tRNAs

Exemplary O-tRNAs comprise, e.g., SEQ ID NO.:1-7 and 12 (See, Table 3). Exemplary O-RSs include, e.g., SEQ ID NOs.: 15 and 16 (See, Table 3). Exemplary polynucleotides that encode O-RSs or portions thereof include, e.g., SEQ ID NOs.: 13 and 14.

Further details of the invention, and in particular experimental details, can be found in Anderson, John Christopher, “Pathway Engineering of the Expanding Genetic Code,” Ph.D. Dissertation, The Scripps Research Institute [2003].

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

TABLE 3

SEQ

| | | |
|---|---|---|
| ID: | Label | SEQUENCE |

| | | |
|---|---|---|
| SEQ ID:1 | HL (TAG) 1 | GCGAGGGTAGCCAAGCTCGGCCAACGGCGACGGACTCTAGATCCGTTCTCGTAG |
| | tRNA | GAGTTCGAGGGTTCGAATCCCTTCCCTCGCACCA |
| SEQ ID:2 | HL (TAG) 2 | GCGAGGGTAGCCAAGCTCGGCCAACGGCGACGGACTCTAAATCCGTTCTCGTAG |
| | tRNA | GAGTTCGAGGGTTCGAATCCCTTCCCTCGCACCA |
| SEQ ID:3 | HL (TAG) 3 | CCCAGGGTAGCCAAGCTCGGCCAACGGCGACGGACTCTAAATCCGTTCTCGTAG |
| | tRNA | GAGTTCGAGGGTTCGAATCCCTTCCCTGGGACCA |
| SEQ ID:4 | HL (AGGA) | GCGAGGGTAGCCAAGCTCGGCCAACGGCGACGGACTTCCTAATCCGTTCTCGTA |
| | 1 | GGAGTTCGAGGGTTCGAATCCCTTCCCTCGCACCA |
| | tRNA | |
| SEQ ID:5 | HL (AGGA) | GCGAGGGTAGCCAAGCTCGGCCAACGGCGACGGACTTCCTAATCCGTTCTCGTA |
| | 2 | GGAGTTCGAGGGTTCGAATCCCTCCCCTCGCACCA |
| | tRNA | |
| SEQ ID:6 | HL (AGGA) | GCGGGGGTTGCCGAGCCTGGCCAAAGGCGCCGGACTTCCTAATCCGGTCCCGTA |
| | 3 | GGGGTTCCGGGGTTCAAATCCCCGCCCCCGCACCA |
| | tRNA | |
| SEQ ID:7 | HL (TGA) 1 | GCGGGGGTTGCCGAGCCTGGCCAAAGGCGCCGGACTTCAAATCCGGTCCCGTAG |
| | tRNA | GGGTTCCGGGGTTCAAATCCCCGCCCCCGCACCA |
| SEQ ID:8 | J17 | CCGGCGGTAGTTCAGCAGGGCAGAACGGCGGACTCTAAATCCGCATGGCGCTGG |
| | M. | TTCAAATCCGGCCCGCCGGACCA |
| | jannaschii | |
| | mtRNA Tyr | |
| | CUA | |
| SEQ ID:9 | SupD | GGAGAGATGCCGGAGCGGCTGAACGGACCGGTCTCTAAAACCGGAGTAGGGGCA |
| | | ACTCTACCGGGGGTTCAAATCCCCCTCTCTCCGCCA |
| SEQ ID:10 | Ser2AGGA | GGAGAGATGCCGGAGCGGCTGAACGGACCGGTCTTCCTAAACCGGAGTAGGGGC |
| | | AACTCTACCGGGGGTTCAAATCCCCCTCTCTCCGCCA |
| SEQ ID:11 | Leu4 of | GCGAGGGUAGCCAAGCUCGGCCAACGGCGACGGACUCAAGAUCCGUUCUCGUAG |
| | Halo | GAGUUCGAGGGUUCGAAUCCCUUCCCUCGCACCA |
| | bacterium | |
| | sp. NRC-1 | |
| SEQ ID:12 | Concensus- | GCGGGGGUUGCCGAGCCUGGCCAAAGGCGCCGGACUUCCUAAUCCCGUCCCGUA |
| | derived | GGGGUUCGGGGGUUCAAAUCCCCGCCCCCGCACCA |
| | AGGA | |
| | Suppressor | |
| SEQ ID:13 | Archaeo | ATGAGCGATT TCAGGATAAT TGAGGAGAAG TGGCAGAAGG CGTGGGAGAA |
| | globus | GGACAGAATT TTTGAGTCCG ATCCTAATGA GAAGGAGAAG TTTTTTCTCA |
| | fulgidus | CAATTCCCTA TCCTTACCTT AATGGAAATC TTCACGCAGG TCACACGAGA |
| | leucyl | ACCTTCACAA TTGGCGATGC CTTCGCCAGA TACATGAGAA TGAAGGGCTA |
| | tRNA- | CAACGTTCTC TTTCCCCTCG GCTTTCATGT TACGGGCACC CCAATCATTG |
| | synthetase | GCCTTGCGGA GCTCATAGCC AAGAGGGACG AGAGGACGAT AGAGGTTTAC |
| | (AFLRS) | ACCAAATACC ATGACGTTCC GCTGGAGGAC TTGCTTCAGC TCACAACTCC |
| | | AGAGAAAATC GTTGAGTACT TCTCAAGGGA GGCGCTGCAG GCTTTGAAGA |
| | | GCATAGGCTA CTCCATTGAC TGGAGGAGGG TTTTCACCAC AACCGATGAA |
| | | GAGTATCAGA GATTCATCGA GTGGCAGTAC TGGAAGCTCA AGGAGCTTGG |
| | | CCTGATTGTG AAGGGCACCC ACCCCGTCAG ATACTGCCCC CACGACCAGA |
| | | ATCCTGTTGA AGACCACGAC CTTCTCGCTG GGGAGGAGGC AACTATTGTT |
| | | GAATTTACCG TTATAAAGTT CAGGCTTGAA GATGGAGACC TCATTTTCCC |
| | | CTGTGCAACT CTCCGTCCCG AAACCGTGTT TGGCGTCACG AACATCTGGG |
| | | TAAAGCCGAC AACCTACGTA ATTGCCGAGG TGGATGGGGA AAAGTGGTTT |
| | | GTGAGCAAAG AGGCTTACGA GAAGCTCACC TACACGGAGA AAAAAGTCAG |
| | | GCTGCTGGAG GAGGTTGATG CGTCGCAGTT CTTCGGCAAG TACGTCATAG |
| | | TCCCGCTGGT AAACAGAAAA GTGCCAATTC TGCCTGCAGA GTTTGTTGAC |
| | | ACCGACAACG CAACAGGAGT TGTGATGAGC GTTCCCGCAC ACGCTCCTTT |
| | | TGACCTGGCT GCCATTGAGG ACTTGAAGAG AGACGAGGAA ACGCTGGCGA |
| | | AGTACGGAAT TGACAAAAGC GTTGTAGAGA GCATAAAGCC AATAGTTCTG |
| | | ATTAAGACGG ACATTGAAGG TGTTCCTGCT GAGAAGCTAA TAAGAGAGCT |
| | | TGGAGTGAAG AGCCAGAAGG ACAAGGAGCT GCTGGATAAG GCAACCAAGA |
| | | CCCTCTACAA GAAGGAGTAC CACACGGGAA TCATGCTGGA CAACACGATG |
| | | AACTATGCTG GAATGAAAGT TTCTGAGGCG AAGGAGAGAG TTCATGAGGA |
| | | TTTGGTTAAG CTTGGCTTGG GGGATGTTTT CTACGAGTTC AGCGAGAAGC |
| | | CCGTAATCTG CAGGTGCGGA ACGAAGTGCG TTGTTAAGGT TGTTAGGGAC |
| | | CAGTGGTTCC TGAACTACTC CAACAGAGAG TGGAAGGAGA AGGTTCTGAA |
| | | TCACCTTGAA AAGATGCGAA TCATCCCCGA CTACTACAAG GAGGAGTTCA |
| | | GGAACAAGAT TGAGTGGCTC AGGGACAAGG CTTGTGCCAG AAGGAAGGGG |
| | | CTTGGAACGA GAATTCCGTG GGATAAGGAG TGGCTCATCG AGAGCCTTTC |
| | | AGACTCAACA ATCTACATGG CCTACTACAT CCTTGCCAAG TACATCAACG |
| | | CAGGATTGCT CAAGGCCGAG AACATGACTC CCGAGTTCCT CGACTACGTG |
| | | CTGCTGGGCA AAGGTGAGGT TGGGAAAGTT GCGGAAGCTT CAAAACTCAG |
| | | CGTGGAGTTA ATCCAGCAGA TCAGGGACGA CTTCGAGTAC TGGTATCCCG |
| | | TTGACCTAAG AAGCAGTGGC AAGGACTTGG TTGCAAACCA CCTGCTCTTC |
| | | TACCTCTTCC ACCACGTCGC CATTTTCCCG CCAGATAAGT GGCCGAGGGC |
| | | AATTGCCGTA AACGGATACG TCAGCCTTGA GGGCAAGAAG ATGAGCAAGA |
| | | GCAAAGGGCC CTTGCTAACG ATGAAGAGGG CGGTGCAGCA GTATOGTGCG |
| | | GATGTGACGA GGCTCTACAT CCTCCACGCT GCAGAGTACG ACAGCGATGC |
| | | GGACTGGAAG AGCAGAGAGG TTGAAGGGCT TGCAAACCAC CTCAGGAGGT |
| | | TCTACAACCT CGTGAAGGAG AACTACCTGA AAGAGGTGGG AGAGCTAACA |
| | | ACCCTCGACC GCTGGCTTGT GAGCAGGATG CAGAGGGCAA TAAAGGAAGT |
| | | GAGGGAGGCT ATGGACAACC TGCAGACGAG GAGGGCCGTG AATGCCGCCT |
| | | TCTTCGAGCT CATGAACGAC GTGAGATGGT ATCTGAGGAG AGGAGGTGAG |
| | | AACCTCGCTA TAATACTGGA CGACTGGATC AAGCTCCTCG CCCCCTTTGC |
| | | TCCGCACATT TGCGAGGAGC TGTGGCACTT GAAGCATGAC AGCTACGTCA |
| | | GCCTCGAAAG CTACCCAGAA TACGACGAAA CCAGGGTTGA CGAGGAGGCG |
| | | GAGAGAATTG AGGAATACCT CCGAAACCTT GTTGAGGACA TTCAGGAAAT |
| | | CAAGAAGTTT GTTAGCGATG CGAAGGAGGT TTACATTGCT CCCGCCGAAG |
| | | ACTGGAAGGT TAAGGCAGCA AAGGTCGTTG CTGAAAGCGG GGATGTTGGG |
| | | GAGGCGATGA AGCAGCTTAT GCAGGACGAG GAGCTTAGGA AGCTCGGCAA |
| | | AGAAGTGTCA AATTTCGTCA AGAAGATTTT CAAAGACAGA AAGAAGCTGA |
| | | TGCTAGTTAA GGAGTGGGAA GTTCTGCAGC AGAACCTGAA ATTTATTGAG |
| | | AATGAGACCG GACTGAAGGT TATTCTTGAT ACTCAGAGAG TTCCTGAGGA |
| | | GAAGAGGAGG CAGGCAGTTC CGGGCAAGCC CGCGATTTAT GTTGCTTAA |
| SEQ ID:14 | Methano | GTGGATATTG AAAGAAAATG GCGTGATAGA TGGAGAGATG CTGGCATATT |
| | bacterium | TCAGGCTGAC CCTGATGACA GAGAAAAGAT ATTCCTCACA GTCGCTTACC |
| | thermo | CCTACCCCAG TGGTGCGATG CACATAGGAC ACGGGAGGAC CTACACTGTC |
| | autotro | CCTGATGTCT ATGCACGGTT CAAGAGGATG CAGGGCTACA ACGTCCTGTT |
| | phicum | TCCCATGGCC TGGCATGTCA CAGGGGCCCC TGTCATAGGG ATAGCGCGGA |
| | leucyl | GGATTCAGAG GAAGGATCCC TGGACCCTCA AAATCTACAG GGAGGTCCAC |
| | tRNA- | AGGGTCCCCG AGGATGAGCT TGAACGTTTC AGTGACCCTG AGTACATAGT |
| | synthetase | TGAATACTTC AGCAGGGAAT ACCGGTCTGT TATGGAGGAT ATGGGCTACT |
| | (MtLRS) | CCATCGACTG GAGGCGTGAA TTCAAAACCA CGGATCCCAC CTACAGCAGG |
| | | TTCATACAGT GGCAGATAAG GAAGCTGAGG GACCTTGGCC TCGTAAGGAA |
| | | GGGCGCCCAT CCTGTTAAGT ACTGCCCTGA ATGTGAAAAC CCTGTGGGTG |
| | | ACCATGACCT CCTTGAGGGT GAGGGGGTTG CCATAAACCA GCTCACACTC |
| | | CTCAAATTCA AACTTGGAGA CTCATACCTG GTCGCAGCCA CCTTCAGGCC |
| | | CGAGACAATC TATGGGGCCA CCAACCTCTG GCTGAACCCT GATGAGGATT |
| | | ATGTGAGGGT TGAAACAGGT GGTGAGGAGT GGATAATAAG CAGGGCTGCC |
| | | GTGGATAATC TTTCACACCA GAAACTGGAC CTCAAGGTTT CCGGTGACGT |
| | | CAACCCCGGG GACCTGATAG GGATGTGCGT GGAGAATCCT GTGACGGGCC |
| | | AGGAACACCC CATACTCCCG GCTTCCTTCG TTGACCCTGA ATATGCCACA |
| | | GGTGTTGTGT TCTCTGTCCC TGCACATGCC CCTGCAGACT TCATAGCCCT |
| | | TGAGGACCTC AGGACAGACC ATGAACTCCT TGAAAGGTAC GGTCTTGAGG |
| | | ATGTGGTTGC TGATATTGAG CCCGTGAATG TCATAGCAGT GGATGGCTAC |
| | | GGTGAGTTCC CGGCGGCCGA GGTTATAGAG AAATTTGGTG TCAGAAACCA |
| | | GGAGGACCCC CGCCTTGAGG ATGCCACCGG GGAGCTATAC AAGATCGAGC |
| | | ATGCGAGGGG TGTTATGAGC AGCCACATCC CTGTCTATGG TGGTATGAAG |
| | | GTCTCTGAGG CCCGTCAGGT CATCGCTGAT GAACTGAAGG ACCAGGGCCT |
| | | TGCAGATGAG ATGTATGAAT TCGCTGAGCG ACCTGTTATA TGCCGCTGCG |
| | | GTGGCAGGTG CGTTGTGAGG GTCATGGAGG ACCAGTGGTT CATGAAGTAC |
| | | TCTGATGACG CCTGGAAGGA CCTCGCCCAC AGGTGCCTCG ATGGCATGAA |
| | | GATAATACCC GAGGAGGTCC GGGCCAACTT TGAATACTAC ATCGACTGGC |
| | | TCAATGACTG GGCATGTTCA AGGAGGATAG GCCTTGGAAC AAGGCTGCCC |
| | | TGGGATGAGA GGTGGATCAT CGAACCCCTC ACAGACTCAA CAATCTACAT |
| | | GGCATATTAC ACCATCGCAC ACCGCCTCAG GGAGATGGAT GCCGGGGAGA |
| | | TGGACGATGA GTTCTTTGAT GCCATATTCC TAGATGATTC AGGAACCTTT |
| | | GAGGATCTCA GGGAGGAATT CCGGTACTGG TACCCCCTTG ACTGGAGGCT |
| | | CTCTGCAAAG GACCTCATAG GCAATCACCT GACATTCCAT ATATTCCACC |
| | | ACTCAGCCAT ATTCCCTGAG TCAGGGTGGC CCCGGGGGGC TGTGGTCTTT |
| | | GGTATGGGCC TTCTTGAGGG CAACAAGATG TCATCCTCCA AGGGCAACGT |
| | | CATACTCCTG AGGGATGCCA TCGAGAAGCA CGGTGCAGAC GTGGTGCGGC |
| | | TCTTCCTCAT GTCCTCAGCA GAGCCATGGC AGGACTTTGA CTGGAGGGAG |
| | | AGTGAGGTCA TCGGGACCCG CAGGAGGATT GAATGGTTCA GGGAATTCGG |
| | | AGAGAGGGTC TCAGGTATCC TGGATGGTAG GCCAGTCCTC AGTGAGGTTA |
| | | CTCCAGCTGA ACCTGAAAGC TTCATTGGAA GGTGGATGAT GGGTCAGCTG |
| | | AACCAGAGGA TACGTGAAGC CACAAGGGCC CTTGAATCAT TCCAGACAAG |
| | | AAAGGCAGTT CAGGAGGCAC TCTATCTCCT TAAAAAGGAT GTTGACCACT |
| | | ACCTTAAGCG TGTTGAGGGT AGAGTTGATG ATGAGGTTAA ATCTGTCCTT |
| | | GCAAACGTTC TGCACGCCTG GATAAGGCTC ATGGCTCCAT TCATACCCTA |
| | | CACTGCTGAG GAGATGTGGG AGAGGTATGG TGGTGAGGGT TTTGTAGCAG |
| | | AAGCTCCATG GCCTGACTTC TCAGATGATG CAGAGAGCAG GGATGTGCAG |
| | | GTTGCAGAGG AGATGGTCCA GAATACCGTT AGAGACATTC AGGAAATCAT |
| | | GAAGATCCTT GGATCCACCC CGGAGAGGGT CCACATATAC ACCTCACCAA |
| | | AATGGAAATG GGATGTGCTA AGGGTCGCAG CAGAGGTAGG AAAACTAGAT |
| | | ATGGGCTCCA TAATGGGAAG GGTTTCAGCT GAGGGCATCC ATGATAACAT |
| | | GAAGGAGGTT GCTGAATTTG TAAGGAGGAT CATCAGGGAC CTTGGTAAAT |
| | | CAGAGGTTAC GGTGATAGAC GAGTACAGCG TACTCATGGA TGCATCTGAT |
| | | TACATTGAAT CAGAGGTTGG AGCCAGGGTT GTGATACACA GCAAACCAGA |
| | | CTATGACCCT GAAAACAAGG CTGTGAATGC CGTTCCCCTG AAGCCAGCCA |
| | | TATACCTTGA ATGA |
| SEQ ID:15 | Archaeo | MSDFRIIEEK WQKAWEKDRI FESDPNEKEK FFLTIPYPYL NGNLHAGHTR |
| | globus | TFTIGDAFAR YMRMKGYNVL FPLGFHVTGT PIIGLAELIA KRDERTIEVY |
| | fulgidus | TKYHDVPLED LLQLTTPEKI VEYFSREALQ ALKSIGYSID WRRVFTTTDE |
| | leucyl | EYQRFIEWQY WKLKELGLIV KGTHPVRYCP HDQNPVEDHD LLAGEEATIV |
| | trna- | EFTVIKFRLE DGDLIFPCAT LRPETVFGVT NIWVKPTTYV IAEVDGEKWF |
| | synthetase | VSKEAYEKLT YTEKKVRLLE EVDASQFFGK YVIVPLVNRK VPILPAEFVD |
| | (AFLRS) | TDNATGVVMS VPAHAPFDLA AIEDLKRDEE TLAKYGIDKS VVESIKPIVL |
| | RS | IKTDIEGVPA EKLIRELGVK SQKDKELLDK ATKTLYKKEY HTGIMLDNTM |
| | | NYAGMKVSEA KERVHEDLVK LGLGDVFYEF SEKPVICRCG TKCVVKVVRD |
| | | QWFLNYSNRE WKEKVLNHLE KMRIIPDYYK EEFRNKIEWL RDKACARRKG |
| | | LGTRIPWDKE WLIESLSDST IYMAYYILAK YINAGLLKAE NMTPEFLDYV |
| | | LLGKGEVGKV AEASKLSVEL IQQIRDDFEY WYPVDLRSSG KDLVANHLLF |
| | | YLFHHVAIFP PDKWPRAIAV NGYVSLEGKK MSKSKGPLLT MKRAVQQYGA |
| | | DVTRLYILHA AEYDSDADWK SREVEGLANH LRRFYNLVKE NYLKEVGELT |
| | | TLDRWLVSRM QRAIKEVREA MDNLQTRRAV NAAFFELMND VRWYLRRGGE |
| | | NLAIILDDWI KLLAPFAPHI CEELWHLKHD SYVSLESYPE YDETRVDEEA |
| | | ERIEEYLRNL VEDIQEIKKF VSDAKEVYIA PAEDWKVKAA KVVAESGDVG |
| | | EAMKQLMQDE ELRKLGKEVS NFVKKIFKDR KKLMLVKEWE VLQQNLKFIE |
| | | NETGLKVILD TQRVPEEKRR QAVPGKPAIY VA* |
| SEQ ID:16 | Methano | VDIERKWRDR WRDAGIFQAD PDDREKIFLT VAYPYPSGAM HIGHGRTYTV |
| | bacterium | PDVYARFKRM QGYNVLFPMA WHVTGAPVIG IARRIQRKDP WTLKIYREVE |
| | thermo | RVPEDELERF SDPEYIVEYF SREYRSVMED MGYSIDWRRE FKTTDPTYSR |
| | autotro | FIQWQIRKLR DLGLVRKGAH PVKYCPECEN PVGDHDLLEG EGVAINQLTL |
| | phicum | LKFKLGDSYL VAATFRPETI YGATNLWLNP DEDYVRVETG GEEWIISRAA |
| | leucyl | VDNLSHQKLD LKVSGDVNPG DLIGMCVENP VTGQEHPILP ASFVDPEYAT |
| | trna- | GVVFSVPAHA PADFIALEDL RTDHELLERY GLEDVVADIE PVNVIAVDGY |
| | synthetase | GEFPAAEVIE KFGVRNQEDP RLEDATGELY KIEHARGVMS SEIPVYGGMK |
| | (MtLRS) | VSEAREVIAD ELKDQGLADE MYEFAERPVI CRCGGRCVVR VMEDQWFMKY |
| | | SDDAWKDLAH RCLDGMKIIP EEVRANFEYY IDWLNDWACS RRIGLGTRLP |
| | | WDERWIIEPL TDSTIYMAYY TIAHRLREMD AGEMDDEFFD AIFLDDSGTF |
| | | EDLREEFRYW YPLDWRLSAK DLIGNHLTFH IFHHSAIFPE SGWPRGAVVF |
| | | GMGLLEGNKM SSSKGNVILL RDAIEKHGAD VVRLFLMSSA EPWQDFDWRE |
| | | SEVIGTRRRI EWFREFGERV SGILDGRPVL SEVTPAEPES FIGRWMMGQL |
| | | NQRIREATRA LESFQTRKAV QEALYLLKKD VDHYLKRVEG RVDDEVKSVL |
| | | ANVLHAWIRL MAPFIPYTAE EMWERYGGEG FVAEAPWPDF SDDAESRDVQ |
| | | VAEEMVQNTV RDIQEIMKIL GSTPERVHIY TSPKWKWDVL RVAAEVGKLD |
| | | MGSIMGRVSA EGIHDNMKEV AEFVRRIIRD LGKSEVTVID EYSVLMDASD |
| | | YIESEVGARV VIHSKPDYDP ENKAVNAVPL KPAIYLE* |
| SEQ ID:17 | pACKO- | gaactccgga tgagcattca tcaggcgggc aagaatgtga ataaaggccg |
| | A184AGGA | gataaaactt gtgcttattt ttctttacgg tctttaaaaa ggccgtaata |
| | | tccagctgaa cggtctggtt ataggtacat tgagcaactg actgaaatgc |
| | | ctcaaaatgt tctttacgat gccattggga tatatcaacg gtggtatatc |
| | | cagtgatttt tttctccatt ttagcttcct tagctcctga aaatctcgat |
| | | aactcaaaaa atacgcccgg tagtgatctt atttcattat ggtgaaagtt |
| | | ggaacctctt acgtgccgat caacgtctca ttttcgccaa aagttggccc |
| | | agggcttccc ggtatcaaca gggacaccag gatttattta ttctgcgaag |
| | | tgatcttccg tcacaggtat ttattcggcg caaagtgcgt cgggtgatgc |
| | | tgccaactta ctgatttagt gtatgatggt gtttttgagg tgctccagtg |
| | | gcttctgttt ctatcagctg tccctcctgt tcagctactg acggggtggt |
| | | gcgtaacggc aaaagcaccg ccggacatca gcgctagcgg agtgtatact |
| | | ggcttactat gttggcactg atgagggtgt cagtgaagtg cttcatgtgg |
| | | caggagaaaa aaggctgcac cggtgcgtca gcagaatatg tgatacagga |
| | | tatattccgc ttcctcgctc actgactcgc tacgctcggt cgttcgactg |
| | | cggcgagcgg aaatggctta cgaacggggc ggagatttcc tggaagatgc |
| | | caggaagata cttaacaggg aagtgagagg gccgcggcaa agccgttttt |
| | | ccataggctc cgcccccctg acaagcatca cgaaatctga cgctcaaatc |
| | | agtggtggcg aaacccgaca ggactataaa gataccaggc gtttccccct |
| | | ggcggctccc tcgtgcgctc tcctgttcct gcctttcggt ttaccggtgt |
| | | cattccgctg ttatggccgc gtttgtctca ttccacgcct gacactcagt |
| | | tccgggtagg cagttcgctc caagctggac tgtatgcacg aaccccccgt |
| | | tcagtccgac cgctgcgcct tatccggtaa ctatcgtctt gagtccaacc |
| | | cggaaagaca tgcaaaagca ccactggcag cagccactgg taattgattt |
| | | agaggagtta gtcttgaagt catgcgccgg ttaaggctaa actgaaagga |
| | | caagttttgg tgactgcgct cctccaagcc agttacctcg gttcaaagag |
| | | ttggtagctc agagaacctt cgaaaaaccg ccctgcaagg cggttttttc |
| | | gttttcagag caagagatta cgcgcagacc aaaacgatct caagaagatc |
| | | atcttattaa tcagataaaa tatttctaga tttcagtgca atttatctct |
| | | tcaaatgtag cacctgaagt cagccccata cgatataagt tgtaattctc |
| | | atgtttgaca gcttatcatc gataagcttt aatgcggtag tttatcacag |
| | | ttaaattgct aacgcagtca ggcaccgtgt atgaaatcta acaatgcgct |
| | | catcgtcatc ctcggcaccg tcaccctgga tgctgtaggc ataggcttgg |
| | | ttatgccggt actgccgggc ctcttgcggg atatcGGTTT CTTAGACGTC |
| | | AGGTGGCACT TTtcggggaa atgtgcgcgg aacccctatt tgtttatttt |
| | | tctaaataca ttcaaatatg tatccgctca tgagacaata accctgataa |
| | | atgcttcaat aatattgaaa aaggaagagt atgagtattc aacatttccg |
| | | tgtcgccctt attccctttt ttgcggcatt ttgccttcct gtttttgctc |
| | | acccagaaac gctggtgaaa gtaaaagatg ctgaagatca gttgggtgca |
| | | cgagtgggtt acatcgaact ggatctcaac agcggtaaga tccttgagag |
| | | ttttcgcccc gaagaacgtt ttccaatgat gagcactttt aaagttctgc |
| | | tatgtggcgc ggtattatcc cgtgttgacg ccgggcaaga gcaactcggt |
| | | cgccgcatac actattctca gaatgacttg gttgagtact caccagtcac |
| | | agaaaagcat cttacggatg gcatgacagt aagagaatta tgcagtgctg |
| | | ccataaccat gagtgataac actgcggcca acttacttct gacaacgatc |
| | | ggaggaccga aggagctaac cgcttttttg cacaacatgg gggatcatgt |
| | | aactcgcctt gatcgttggg aaccggagct gaatgaagcc ataccaaacg |
| | | acgagcgtga caccacgatg cctAGGAgca atggcaacaa cgttgcgcaa |
| | | actattaact ggcgaactac ttactctagc ttcccggcaa caattaatag |
| | | actggatgga ggcggataaa gttgcaggac cacttctgcg ctcggccctt |
| | | ccggctggct ggtttattgc tgataaatct ggagccggtg agcgtgggtc |
| | | tcgcggtatc attgcagcac tggggccaga tggtaagccc tcccgtatcg |
| | | tagttatcta cacgacgggg agtcaggcaa ctatggatga acgaaataga |
| | | cagatcgctg agataggtgc ctcactgatt aagcattggc accaccacca |
| | | ccaccactaa CCCGGGACCA AGTTTACTCA TATATACttt agattgattt |
| | | aaaacttcat ttttaattta aaaggatcta ggtgaagatc ctttttgata |
| | | atCTCATGAC CAAAATCCCT TAACGgcatg caccattcct tgcggcggcg |
| | | gtgctcaacg gcctcaacct actactGGGC TGCTTCCTAA TGCAGGAGTC |
| | | GCATAAGGGA GAGCGTCTGG CGAAAGGGGG ATGTGCTGCA AGGCGATTAA |
| | | GTTGGGTAAC GCCAGGGTTT TCCCAGTCAC GACGTTGTAA AACGACGGCC |
| | | AGTGCCAAGC TTAAAAAaaa tccttagctt tcgctaagga tCTGCAGTTA |
| | | TAATCTCTTT CTAATTGGCT CTAAAATCTT TATAAGTTCT TCAGCTACAG |
| | | CATTTTTTAA ATCCATTGGA TGCAATTCCT TATTTTTAAA TAAACTCTCT |
| | | AACTCCTCAT AGCTATTAAC TGTCAAATCT CCACCAAATT TTTCTGGCCT |
| | | TTTTATGGTT AAAGGATATT CAAGGAAGTA TTTAGCTATC TCCATTATTG |
| | | GATTTCCTTC AACAACTCCA GCTGGGCAGT ATGCTTTCTT TATCTTAGCC |
| | | CTAATCTCTT CTGGAGAGTC ATCAACAGCT ATAAAATTCC CTTTTGAAGA |
| | | ACTCATCTTT CCTTCTCCAT CCAAACCCGT TAAGACAGGG TTGTGAATAC |
| | | AAACAACCTT TTTTGGTAAA AGCTCCCTTG CTAACATGTG TATTTTTCTC |
| | | TGCTCCATCC CTCCAACTGC AACATCAACG CCTAAATAAT GAATATCATT |
| | | AACCTGCATT ATTGGATAGA TAACTTCACC AACCTTTGGA TTTTCATCCT |
| | | CTCTTGCTAT AAGTTCCATA CTCCTTCTTG CTCTTTTTAA GGTAGTTTTT |
| | | AAAGCCAATC TATAGACATT CAGTGTATAA TCCTTATCAA GCTGGAATTC |
| | | agcgttacaa gtattacaca aagtttttta tgttgagaat atttttttga |
| | | tggggcgcca cttatttttg atcgttcgct caaagAAGCG GCGCCAGGGN |
| | | TGTTTTTCTT TTCACCAGTN AGACGGGCAA CAGAACGCCA TGAgcggcct |
| | | catttcttat tctgagttac aacagtccgc accgctgtcc ggtagctcct |
| | | tccggtgggc gcggggcatg actatcgtcg ccgcacttat gactgtcttc |
| | | tttatcatgc aactcgtagg acaggtgccg gcagcgccca acagtccccc |
| | | ggccacgggg cctgccacca tacccacgcc gaaacaagcg ccctgcacca |
| | | ttatgttccg gatctgcatc gcaggatgct gctggctacc ctgtggaaca |
| | | cctacatctg tattaacgaa gcgctaaccg tttttatcag gctctgggag |
| | | gcagaataaa tgatcatatc gtcaattatt acctccacgg ggagagcctg |
| | | agcaaactgg cctcaggcat ttgagaagca cacggtcaca ctgcttccgg |
| | | tagtcaataa accggtaaac cagcaataga cataagcggc tatttaacga |
| | | ccctgccctg aaccgacgac cgggtcgaat ttgctttcga atttctgcca |
| | | ttcatccgct tattatcact tattcaggcg tagcaccagg cgtttaaggg |
| | | caccaataac tgccttaaaa aaattacgcc ccgccctgcc actcatcgca |
| | | gtactgttgt aattcattaa gcattctgcc gacatggaag ccatcacaga |
| | | cggcatgatg aacctgaatc gccagcggca tcagcacctt gtcgccttgc |
| | | gtataatatt tgcccatggt gaaaacgggg gcgaagaagt tgtccatatt |
| | | ggccacgttt aaatcaaaac tggtgaaact cacccaggga ttggctgaga |
| | | cgaaaaacat attctcaata aaccctttag ggaaataggc caggttttca |
| | | ccgtaacacg ccacatcttg cgaatatatg tgtagaaact gccggaaatc |
| | | gtcgtggtat tcactccaga gcgatgaaaa cgtttcagtt tgctcatgga |
| | | aaacggtgta acaagggtga acactatccc atatcaccag ctcaccgtct |
| | | ttcattgcca tacg |
| SEQ ID:18 | pACKO- | gaactccgga tgagcattca tcaggcgggc aagaatgtga ataaaggccg |
| | A184TAG | gataaaactt gtgcttattt ttctttacgg tctttaaaaa ggccgtaata |
| | | tccagctgaa cggtctggtt ataggtacat tgagcaactg actgaaatgc |
| | | ctcaaaatgt tctttacgat gccattggga tatatcaacg gtggtatatc |
| | | cagtgatttt tttctccatt ttagcttcct tagctcctga aaatctcgat |
| | | aactcaaaaa atacgcccgg tagtgatctt atttcattat ggtgaaagtt |
| | | ggaacctctt acgtgccgat caacgtctca ttttcgccaa aagttggccc |
| | | agggcttccc ggtatcaaca gggacaccag gatttattta ttctgcgaag |
| | | tgatcttccg tcacaggtat ttattcggcg caaagtgcgt cgggtgatgc |
| | | tgccaactta ctgatttagt gtatgatggt gtttttgagg tgctccagtg |
| | | gcttctgttt ctatcagctg tccctcctgt tcagctactg acggggtggt |
| | | gcgtaacggc aaaagcaccg ccggacatca gcgctagcgg agtgtatact |
| | | ggcttactat gttggcactg atgagggtgt cagtgaagtg cttcatgtgg |
| | | caggagaaaa aaggctgcac cggtgcgtca gcagaatatg tgatacagga |
| | | tatattccgc ttcctcgctc actgactcgc tacgctcggt cgttcgactg |
| | | cggcgagcgg aaatggctta cgaacggggc ggagatttcc tggaagatgc |
| | | caggaagata cttaacaggg aagtgagagg gccgcggcaa agccgttttt |
| | | ccataggctc cgcccccctg acaagcatca cgaaatctga cgctcaaatc |
| | | agtggtggcg aaacccgaca ggactataaa gataccaggc gtttccccct |
| | | ggcggctccc tcgtgcgctc tcctgttcct gcctttcggt ttaccggtgt |
| | | cattccgctg ttatggccgc gtttgtctca ttccacgcct gacactcagt |
| | | tccgggtagg cagttcgctc caagctggac tgtatgcacg aaccccccgt |
| | | tcagtccgac cgctgcgcct tatccggtaa ctatcgtctt gagtccaacc |
| | | cggaaagaca tgcaaaagca ccactggcag cagccactgg taattgattt |
| | | agaggagtta gtcttgaagt catgcgccgg ttaaggctaa actgaaagga |
| | | caagttttgg tgactgcgct cctccaagcc agttacctcg gttcaaagag |
| | | ttggtagctc agagaacctt cgaaaaaccg ccctgcaagg cggttttttc |
| | | gttttcagag caagagatta cgcgcagacc aaaacgatct caagaagatc |
| | | atcttattaa tcagataaaa tatttctaga tttcagtgca atttatctct |
| | | tcaaatgtag cacctgaagt cagccccata cgatataagt tgtaattctc |
| | | atgtttgaca gcttatcatc gataagcttt aatgcggtag tttatcacag |
| | | ttaaattgct aacgcagtca ggcaccgtgt atgaaatcta acaatgcgct |
| | | catcgtcatc ctcggcaccg tcaccctgga tgctgtaggc ataggcttgg |
| | | ttatgccggt actgccgggc ctcttgcggg atatcGGTTT CTTAGACGTC |
| | | AGGTGGCACT TTtcggggaa atgtgcgcgg aacccctatt tgtttatttt |
| | | tctaaataca ttcaaatatg tatccgctca tgagacaata accctgataa |
| | | atgcttcaat aatattgaaa aaggaagagt atgagtattc aacatttccg |
| | | tgtcgccctt attccctttt ttgcggcatt ttgccttcct gtttttgctc |
| | | acccagaaac gctggtgaaa gtaaaagatg ctgaagatca gttgggtgca |
| | | cgagtgggtt acatcgaact ggatctcaac agcggtaaga tccttgagag |
| | | ttttcgcccc gaagaacgtt ttccaatgat gagcactttt aaagttctgc |
| | | tatgtggcgc ggtattatcc cgtgttgacg ccgggcaaga gcaactcggt |
| | | cgccgcatac actattctca gaatgacttg gttgagtact caccagtcac |
| | | agaaaagcat cttacggatg gcatgacagt aagagaatta tgcagtgctg |
| | | ccataaccat gagtgataac actgcggcca acttacttct gacaacgatc |
| | | ggaggaccga aggagctaac cgcttttttg cacaacatgg gggatcatgt |
| | | aactcgcctt gatcgttggg aaccggagct gaatgaagcc ataccaaacg |
| | | acgagcgtga caccacgatg cctTAGgcaa tggcaacaac gttgcgcaaa |
| | | ctattaactg gcgaactact tactctagct tcccggcaac aattaataga |
| | | ctggatggag gcggataaag ttgcaggacc acttctgcgc tcggcccttc |
| | | cggctggctg gtttattgct gataaatctg gagccggtga gcgtgggtct |
| | | cgcggtatca ttgcagcact ggggccagat ggtaagccct cccgtatcgt |
| | | agttatctac acgacgggga gtcaggcaac tatggatgaa cgaaatagac |
| | | agatcgctga gataggtgcc tcactgatta agcattggca ccaccaccac |
| | | caccactaaC CCGGGACCAA GTTTACTCAT ATATACttta gattgattta |
| | | aaacttcatt tttaatttaa aaggatctag gtgaagatcc tttttgataa |
| | | tCTCATGACC AAAATCCCTT AACGgcatgc accattcctt gcggcggcgg |
| | | tgctcaacgg cctcaaccta ctactGGGCT GCTTCCTAAT GCAGGAGTCG |
| | | CATAAGGGAG AGCGTCTGGC GAAAGGGGGA TGTGCTGCAA CGCGATTAAG |
| | | TTGGGTAACG CCAGGGTTTT CCCAGTCACG ACGTTGTAAA ACGACGGCCA |
| | | GTGCCAAGCT TAAAAAaaat ccttagcttt cgctaaggat CTGCAGTTAT |
| | | AATCTCTTTC TAATTGGCTC TAAAATCTTT ATAAGTTCTT CAGCTACAGC |
| | | ATTTTTTAAA TCCATTGGAT GCAATTCCTT ATTTTTAAAT AAACTCTCTA |
| | | ACTCCTCATA GCTATTAACT GTCAAATCTC CACCAAATTT TTCTGGCCTT |
| | | TTTATGGTTA AAGGATATTC AAGGAAGTAT TTAGCTATCT CCATTATTGG |
| | | ATTTCCTTCA ACAACTCCAG CTGGGCAGTA TGCTTTCTTT ATCTTAGCCC |
| | | TAATCTCTTC TGGAGAGTCA TCAACAGCTA TAAAATTCCC TTTTGAAGAA |
| | | CTCATCTTTC CTTCTCCATC CAAACCCGTT AAGACAGGGT TGTGAATACA |
| | | AACAACCTTT TTTGGTAAAA GCTCCCTTGC TAACATGTGT ATTTTTCTCT |
| | | GCTCCATCCC TCCAACTGCA ACATCAACGC CTAAATAATG AATATCATTA |
| | | ACCTGCATTA TTGGATAGAT AACTTCAGCA ACCTTTGGAT TTTCATCCTC |
| | | TCTTGCTATA AGTTCCATAC TCCTTCTTGC TCTTTTTAAG GTAGTTTTTA |
| | | AAGCCAATCT ATAGACATTC AGTGTATAAT CCTTATCAAG CTGGAATTCa |
| | | gcgttacaag tattacacaa agttttttat gttgagaata tttttttgat |
| | | ggggcgccac ttatttttga tcgttcgctc aaagAAGCGG CGCCAGGGNT |
| | | GTTTTTCTTT TCACCAGTNA GACGGGCAAC AGAACGCCAT Gagcggcctc |
| | | atttcttatt ctgagttaca acagtccgca ccgctgtccg gtagctcctt |
| | | ccggtgggcg cggggcatga ctatcgtcgc cgcacttatg actgtcttct |
| | | ttatcatgca actcgtagga caggtgccgg cagcgcccaa cagtcccccg |
| | | gccacggggc ctgccaccat acccacgccg aaacaagcgc cctgcaccat |
| | | tatgttccgg atctgcatcg caggatgctg ctggctaccc tgtggaacac |
| | | ctacatctgt attaacgaag cgctaaccgt ttttatcagg ctctgggagg |
| | | cagaataaat gatcatatcg tcaattatta cctccacggg gagagcctga |
| | | gcaaactggc ctcaggcatt tgagaagcac acggtcacac tgcttccggt |
| | | agtcaataaa ccggtaaacc agcaatagac ataagcggct atttaacgac |
| | | cctgccctga accgacgacc gggtcgaatt tgctttcgaa tttctgccat |
| | | tcatccgctt attatcactt attcaggcgt agcaccaggc gtttaagggc |
| | | accaataact gccttaaaaa aattacgccc cgccctgcca ctcatcgcag |
| | | tactgttgta attcattaag cattctgccg acatggaagc catcacagac |
| | | ggcatgatga acctgaatcg ccagcggcat cagcaccttg tcgccttgcg |
| | | tataatattt gcccatggtg aaaacggggg cgaagaagtt gtccatattg |
| | | gccacgttta aatcaaaact ggtgaaactc acccagggat tggctgagac |
| | | gaaaaacata ttctcaataa accctttagg gaaataggcc aggttttcac |
| | | cgtaacacgc cacatcttgc gaatatatgt gtagaaactg ccggaaatcg |
| | | tcgtggtatt cactccagag cgatgaaaac gtttcagttt gctcatggaa |
| | | aacggtgtaa caagggtgaa cactatccca tatcaccagc tcaccgtctt |
| | | tcattgccat acg |
| SEQ ID:19 | pACKO- | gaactccgga tgagcattca tcaggcgggc aagaatgtga ataaaggccg |
| | A184TGA | gataaaactt gtgcttattt ttctttacgg tctttaaaaa ggccgtaata |
| | | tccagctgaa cggtctggtt ataggtacat tgagcaactg actgaaatgc |
| | | ctcaaaatgt tctttacgat gccattggga tatatcaacg gtggtatatc |
| | | cagtgatttt tttctccatt ttagcttcct tagctcctga aaatctcgat |
| | | aactcaaaaa atacgcccgg tagtgatctt atttcattat ggtgaaagtt |
| | | ggaacctctt acgtgccgat caacgtctca ttttcgccaa aagttggccc |
| | | agggcttccc ggtatcaaca gggacaccag gatttattta ttctgcgaag |
| | | tgatcttccg tcacaggtat ttattcggcg caaagtgcgt cgggtgatgc |
| | | tgccaactta ctgatttagt gtatgatggt gtttttgagg tgctccagtg |
| | | gcttctgttt ctatcagctg tccctcctgt tcagctactg acggggtggt |
| | | gcgtaacggc aaaagcaccg ccggacatca gcgctagcgg agtgtatact |
| | | ggcttactat gttggcactg atgagggtgt cagtgaagtg cttcatgtgg |
| | | caggagaaaa aaggctgcac cggtgcgtca gcagaatatg tgatacagga |
| | | tatattccgc ttcctcgctc actgactcgc tacgctcggt cgttcgactg |
| | | cggcgagcgg aaatggctta cgaacggggc ggagatttcc tggaagatgc |
| | | caggaagata cttaacaggg aagtgagagg gccgcggcaa agccgttttt |
| | | ccataggctc cgcccccctg acaagcatca cgaaatctga cgctcaaatc |
| | | agtggtggcg aaacccgaca ggactataaa gataccaggc gtttccccct |
| | | ggcggctccc tcgtgcgctc tcctgttcct gcctttcggt ttaccggtgt |
| | | cattccgctg ttatggccgc gtttgtctca ttccacgcct gacactcagt |
| | | tccgggtagg cagttcgctc caagctggac tgtatgcacg aaccccccgt |
| | | tcagtccgac cgctgcgcct tatccggtaa ctatcgtctt gagtccaacc |
| | | cggaaagaca tgcaaaagca ccactggcag cagccactgg taattgattt |
| | | agaggagtta gtcttgaagt catgcgccgg ttaaggctaa actgaaagga |
| | | caagttttgg tgactgcgct cctccaagcc agttacctcg gttcaaagag |
| | | ttggtagctc agagaacctt cgaaaaaccg ccctgcaagg cggttttttc |
| | | gttttcagag caagagatta cgcgcagacc aaaacgatct caagaagatc |
| | | atcttattaa tcagataaaa tatttctaga tttcagtgca atttatctct |
| | | tcaaatgtag cacctgaagt cagccccata cgatataagt tgtaattctc |
| | | atgtttgaca gcttatcatc gataagcttt aatgcggtag tttatcacag |
| | | ttaaattgct aacgcagtca ggcaccgtgt atgaaatcta acaatgcgct |
| | | catcgtcatc ctcggcaccg tcaccctgga tgctgtaggc ataggcttgg |
| | | ttatgccggt actgccgggc ctcttgcggg atatcGGTTT CTTAGACGTC |
| | | AGGTGGCACT TTtcggggaa atgtgcgcgg aacccctatt tgtttatttt |
| | | tctaaataca ttcaaatatg tatccgctca tgagacaata accctgataa |
| | | atgcttcaat aatattgaaa aaggaagagt atgagtattc aacatttccg |
| | | tgtcgccctt attccctttt ttgcggcatt ttgccttcct gtttttgctc |
| | | acccagaaac gctggtgaaa gtaaaagatg ctgaagatca gttgggtgca |
| | | cgagtgggtt acatcgaact ggatctcaac agcggtaaga tccttgagag |
| | | ttttcgcccc gaagaacgtt ttccaatgat gagcactttt aaagttctgc |
| | | tatgtggcgc ggtattatcc cgtgttgacg ccgggcaaga gcaactcggt |
| | | cgccgcatac actattctca gaatgacttg gttgagtact caccagtcac |
| | | agaaaagcat cttacggatg gcatgacagt aagagaatta tgcagtgctg |
| | | ccataaccat gagtgataac actgcggcca acttacttct gacaacgatc |
| | | ggaggaccga aggagctaac cgcttttttg cacaacatgg gggatcatgt |
| | | aactcgcctt gatcgttggg aaccggagct gaatgaagcc ataccaaacg |
| | | acgagcgtga caccacgatg cctTGAgcaa tggcaacaac gttgcgcaaa |
| | | ctattaactg gcgaactact tactctagct tcccggcaac aattaataga |
| | | ctggatggag gcggataaag ttgcaggacc acttctgcgc tcggcccttc |
| | | cggctggctg gtttattgct gataaatctg gagccggtga gcgtgggtct |
| | | cgcggtatca ttgcagcact ggggccagat ggtaagccct cccgtatcgt |
| | | agttatctac acgacgggga gtcaggcaac tatggatgaa cgaaatagac |
| | | agatcgctga gataggtgcc tcactgatta agcattggca ccaccaccac |
| | | caccactaaC CCGGGACCAA GTTTACTCAT ATATACttta gattgattta |
| | | aaacttcatt tttaatttaa aaggatctag gtgaagatcc tttttgataa |
| | | tCTCATGACC AAAATCCCTT AACGgcatgc accattcctt gcggcggcgg |
| | | tgctcaacgg cctcaaccta ctactGGGCT GCTTCCTAAT GCAGGAGTCG |
| | | CATAAGGGAG AGCGTCTGGC GAAAGGGGGA TGTGCTGCAA GGCGATTAAG |
| | | TTGGGTAACG CCAGGGTTTT CCCAGTCACG ACGTTGTAAA ACGACGGCCA |
| | | GTGCCAAGCT TAAAAAaaat ccttagcttt cgctaaggat CTGCAGTTAT |
| | | AATCTCTTTC TAATTGGCTC TAAAATCTTT ATAAGTTCTT CAGCTACAGC |
| | | ATTTTTTAAA TCCATTGGAT GCAATTCCTT ATTTTTAAAT AAACTCTCTA |
| | | ACTCCTCATA GCTATTAACT GTCAAATCTC CACCAAATTT TTCTGGCCTT |
| | | TTTATGGTTA AAGGATATTC AAGGAAGTAT TTAGCTATCT CCATTATTGG |
| | | ATTTCCTTCA ACAACTCCAG CTGGGCAGTA TGCTTTCTTT ATCTTAGCCC |
| | | TAATCTCTTC TGCAGAGTCA TCAACAGCTA TAAAATTCCC TTTTGAAGAA |
| | | CTCATCTTTC CTTCTCCATC CAAACCCGTT AAGACAGGGT TGTGAATACA |
| | | AACAACCTTT TTTGGTAAAA GCTCCCTTGC TAACATGTGT ATTTTTCTCT |
| | | GCTCCATCCC TCCAACTGCA ACATCAACGC CTAAATAATG AATATCATTA |
| | | ACCTGCATTA TTGGATAGAT AACTTCAGCA ACCTTTGGAT TTTCATCCTC |
| | | TCTTGCTATA AGTTCCATAC TCCTTCTTGC TCTTTTTAAG GTAGTTTTTA |
| | | AAGCCAATCT ATAGACATTC AGTGTATAAT CCTTATCAAG CTGGAATTCa |
| | | gcgttacaag tattacacaa agttttttat gttgagaata tttttttgat |
| | | ggggcgccac ttatttttga tcgttcgctc aaagAAGCGG CGCCAGGGNT |
| | | GTTTTTCTTT TCACCAGTNA GACGGGCAAC AGAACGCCAT Gagcggcctc |
| | | atttcttatt ctgagttaca acagtccgca ccgctgtccg gtagctcctt |
| | | ccggtgggcg cggggcatga ctatcgtcgc cgcacttatg actgtcttct |
| | | ttatcatgca actcgtagga caggtgccgg cagcgcccaa cagtcccccg |
| | | gccacggggc ctgccaccat acccacgccg aaacaagcgc cctgcaccat |
| | | tatgttccgg atctgcatcg caggatgctg ctggctaccc tgtggaacac |
| | | ctacatctgt attaacgaag cgctaaccgt ttttatcagg ctctgggagg |
| | | cagaataaat gatcatatcg tcaattatta cctccacggg gagagcctga |
| | | gcaaactggc ctcaggcatt tgagaagcac acggtcacac tgcttccggt |
| | | agtcaataaa ccggtaaacc agcaatagac ataagcggct atttaacgac |
| | | cctgccctga accgacgacc gggtcgaatt tgctttcgaa tttctgccat |
| | | tcatccgctt attatcactt attcaggcgt agcaccaggc gtttaagggc |
| | | accaataact gccttaaaaa aattacgccc cgccctgcca ctcatcgcag |
| | | tactgttgta attcattaag cattctgccg acatggaagc catcacagac |
| | | ggcatgatga acctgaatcg ccagcggcat cagcaccttg tcgccttgcg |
| | | tataatattt gcccatggtg aaaacggggg cgaagaagtt gtccatattg |
| | | gccacgttta aatcaaaact ggtgaaactc acccagggat tggctgagac |
| | | gaaaaacata ttctcaataa accctttagg gaaataggcc aggttttcac |
| | | cgtaacacgc cacatcttgc gaatatatgt gtagaaactg ccggaaatcg |
| | | tcgtggtatt cactccagag cgatgaaaac gtttcagttt gctcatggaa |
| | | aacggtgtaa caagggtgaa cactatccca tatcaccagc tcaccgtctt |
| | | tcattgccat acg |
| SEQ ID:20 | pACKO- | gaactccgga tgagcattca tcaggcgggc aagaatgtga ataaaggccg |
| | Bla | gataaaactt gtgcttattt ttctttacgg tctttaaaaa ggccgtaata |
| | | tccagctgaa cggtctggtt ataggtacat tgagcaactg actgaaatgc |
| | | ctcaaaatgt tctttacgat gccattggga tatatcaacg gtggtatatc |
| | | cagtgatttt tttctccatt ttagcttcct tagctcctga aaatctcgat |
| | | aactcaaaaa atacgcccgg tagtgatctt atttcattat ggtgaaagtt |
| | | ggaacctctt acgtgccgat caacgtctca ttttcgccaa aagttggccc |
| | | agggcttccc ggtatcaaca gggacaccag gatttattta ttctgcgaag |
| | | tgatcttccg tcacaggtat ttattcggcg caaagtgcgt cgggtgatgc |
| | | tgccaactta ctgatttagt gtatgatggt gtttttgagg tgctccagtg |
| | | gcttctgttt ctatcagctg tccctcctgt tcagctactg acggggtggt |
| | | gcgtaacggc aaaagcaccg ccggacatca gcgctagcgg agtgtatact |
| | | ggcttactat gttggcactg atgagggtgt cagtgaagtg cttcatgtgg |
| | | caggagaaaa aaggctgcac cggtgcgtca gcagaatatg tgatacagga |
| | | tatattccgc ttcctcgctc actgactcgc tacgctcggt cgttcgactg |
| | | cggcgagcgg aaatggctta cgaacggggc ggagatttcc tggaagatgc |
| | | caggaagata cttaacaggg aagtgagagg gccgcggcaa agccgttttt |
| | | ccataggctc cgcccccctg acaagcatca cgaaatctga cgctcaaatc |
| | | agtggtggcg aaacccgaca ggactataaa gataccaggc gtttccccct |
| | | ggcggctccc tcgtgcgctc tcctgttcct gcctttcggt ttaccggtgt |
| | | cattccgctg ttatggccgc gtttgtctca ttccacgcct gacactcagt |
| | | tccgggtagg cagttcgctc caagctggac tgtatgcacg aaccccccgt |
| | | tcagtccgac cgctgcgcct tatccggtaa ctatcgtctt gagtccaacc |
| | | cggaaagaca tgcaaaagca ccactggcag cagccactgg taattgattt |
| | | agaggagtta gtcttgaagt catgcgccgg ttaaggctaa actgaaagga |
| | | caagttttgg tgactgcgct cctccaagcc agttacctcg gttcaaagag |
| | | ttggtagctc agagaacctt cgaaaaaccg ccctgcaagg cggttttttc |
| | | gttttcagag caagagatta cgcgcagacc aaaacgatct caagaagatc |
| | | atcttattaa tcagataaaa tatttctaga tttcagtgca atttatctct |
| | | tcaaatgtag cacctgaagt cagccccata cgatataagt tgtaattctc |
| | | atgtttgaca gcttatcatc gataagcttt aatgcggtag tttatcacag |
| | | ttaaattgct aacgcagtca ggcaccgtgt atgaaatcta acaatgcgct |
| | | catcgtcatc ctcggcaccg tcaccctgga tgctgtaggc ataggcttgg |
| | | ttatgccggt actgccgggc ctcttgcggg atatcGGTTT CTTAGACGTC |
| | | AGGTGGCact tttcggggaa atgtgcgcgg aacccctatt tgtttatttt |
| | | tctaaataca ttcaaatatg tatccgctca tgagacaata accctgataa |
| | | atgcttcaat aatattgaaa aaggaagagt atgagtattc aacatttccg |
| | | tgtcgccctt attccctttt ttgcggcatt ttgccttCCT GTTTTTGCTC |
| | | ACCCAGAAAC ACTAGtgcag caatggcaac aacgttgcgc aaactattaa |
| | | ctggcgaact acttactcta gcttcccggc aacaattaat agactggatg |
| | | gaggcggata aagttgcagg accacttctg cgctcggccc ttccggctgg |
| | | ctggtttatt gctgataaat ctggagccgg tgagcgtggg tctcgcggta |
| | | tcattgcagc actggggcca gatggtaagc cctcccgtat cgtagttatc |
| | | tacacgacgg ggagtcaggc aactatggat gaacgaaata gacagatcgc |
| | | tgagataggt gcctCACTGA TTAAGCATTG GTAACCCGGG ACCAAGTTTA |
| | | CTCATATATA Ctttagattg atttaaaact tcatttttaa tttaaaagga |
| | | tctaggtgaa gatccttttt gataatCTCA TGACCAAAAT CCCTTAACGg |
| | | catgcaccat tccttgcggc ggcggtgctc aacggcctca acctactact |
| | | GGGCTGCTTC CTAATGCAGG AGTCGCATAA GGGAGAGCGT CTGGCGAAAG |
| | | GGGGATGTGC TGCAAGGCGA TTAAGTTGGG TAACGCCAGG GTTTTCCCAG |
| | | TCACGACGTT GTAAAACGAC GGCCAGTGCC AAGCTTAAAA Aaaatcctta |
| | | gctttcgcta aggatCTGCA GTTATAATCT CTTTCTAATT GGCTCTAAAA |
| | | TCTTTATAAG TTCTTCAGCT ACAGCATTTT TTAAATCCAT TGGATGCAAT |
| | | TCCTTATTTT TAAATAAACT CTCTAACTCC TCATAGCTAT TAACTGTCAA |
| | | ATCTCCACCA AATTTTTCTG GCCTTTTTAT GGTTAAAGGA TATTCAAGGA |
| | | AGTATTTAGC TATCTCCATT ATTGGATTTC CTTCAACAAC TCCAGCTGGG |
| | | CAGTATGCTT TCTTTATCTT AGCCCTAATC TCTTCTGGAG AGTCATCAAC |
| | | AGCTATAAAA TTCCCTTTTG AAGAACTCAT CTTTCCTTCT CCATCCAAAC |
| | | CCGTTAAGAC AGGGTTGTGA ATACAAACAA CCTTTTTTGG TAAAAGCTCC |
| | | CTTGCTAACA TGTGTATTTT TCTCTGCTCC ATCCCTCCAA CTGCAACATC |
| | | AACGCCTAAA TAATGAATAT CATTAACCTG CATTATTGGA TAGATAACTT |
| | | CAGCAACCTT TGGATTTTCA TCCTCTCTTG CTATAAGTTC CATACTCCTT |
| | | CTTGCTCTTT TTAAGGTAGT TTTTAAAGCC AATCTATAGA CATTCAGTGT |
| | | ATAATCCTTA TCAAGCTGGA ATTCagcgtt acaagtatta cacaaagttt |
| | | tttatgttga gaatattttt ttgatggggc gccacttatt tttgatcgtt |
| | | cgctcaaagA AGCGGCGCCA GGGNTGTTTT TCTTTTCACC AGTNAGACGG |
| | | GCAACAGAAC GCCATGAgcg gcctcatttc ttattctgag ttacaacagt |
| | | ccgcaccgct gtccggtagc tccttccggt gggcgcgggg catgactatc |
| | | gtcgccgcac ttatgactgt cttctttatc atgcaactcg taggacaggt |
| | | gccggcagcg cccaacagtc ccccggccac ggggcctgcc accataccca |
| | | cgccgaaaca agcgccctgc accattatgt tccggatctg catcgcagga |
| | | tgctgctggc taccctgtgg aacacctaca tctgtattaa cgaagcgcta |
| | | accgttttta tcaggctctg ggaggcagaa taaatgatca tatcgtcaat |
| | | tattacctcc acggggagag cctgagcaaa ctggcctcag gcatttgaga |
| | | agcacacggt cacactgctt ccggtagtca ataaaccggt aaaccagcaa |
| | | tagacataag cggctattta acgaccctgc cctgaaccga cgaccgggtc |
| | | gaatttgctt tcgaatttct gccattcatc cgcttattat cacttattca |
| | | ggcgtagcac caggcgttta agggcaccaa taactgcctt aaaaaaatta |
| | | cgccccgccc tgccactcat cgcagtactg ttgtaattca ttaagcattc |
| | | tgccgacatg gaagccatca cagacggcat gatgaacctg aatcgccagc |
| | | ggcatcagca ccttgtcgcc ttgcgtataa tatttgccca tggtgaaaac |
| | | gggggcgaag aagttgtcca tattggccac gtttaaatca aaactggtga |
| | | aactcaccca gggattggct gagacgaaaa acatattctc aataaaccct |
| | | ttagggaaat aggccaggtt ttcaccgtaa cacgccacat cttgcgaata |
| | | tatgtgtaga aactgccgga aatcgtcgtg gtattcactc cagagcgatg |
| | | aaaacgtttc agtttgctca tggaaaacgg tgtaacaagg gtgaacacta |
| | | tcccatatca ccagctcacc gtctttcatt gccatacg |
| SEQ ID:21 | pKQ | ATGGATCCGA GCTCGAGATC TGCAGCTGGT ACCATATGGG AATTCGAAGC |
| | | TTGGGCCCGA ACAAAAACTC ATCTCAGAAG AGGATCTGAA TAGCGCCGTC |
| | | GACCATCATC ATCATCATCA TTGAGTTTAA ACGGTCTCCA GCTTGGCTGT |
| | | TTTGGCGGAT GAGAGAAGAT TTTCAGCCTG ATACAGATTA AATCAGAACG |
| | | CAGAAGCGGT CTGATAAAAC AGAATTTGCC TGGCGGCAGT AGCGCGGTGG |
| | | TCCCACCTGA CCCCATGCCG AACTCAGAAG TGAAACGCCG TAGCGCCGAT |
| | | GGTAGTGTGG GGTCTCCCCA TGCGAGAGTA GGGAACTGCC AGGCATCAAA |
| | | TAAAACGAAA GGCTCAGTCG AAAGACTGGG CCTTTCGTTT TATCTGTTGT |
| | | TTGTCGGTGA ACGATATCTG CTTTTCTTCG CGAATtaatt ccgcttcgca |
| | | ACATGTgagc aaaaggccag caaaaggcca ggaaccgtaa aaaggccgcg |
| | | ttgctggcgt ttttccatag gctccgcccc cctgacgagc atcacaaaaa |
| | | tcgacgctca agtcagaggt ggcgaaaccc gacaggacta taaagatacc |
| | | aggcgtttcc ccctggaagc tccctcgtgc gctctcctgt tccgaccctg |
| | | ccgcttaccg gatacctgtc cgcctttctc ccttcgggaa gcgtggcgct |
| | | ttctcatagc tcacgctgta ggtatctcag ttcggtgtag gtcgttcgct |
| | | ccaagctggg ctgtgtgcac gaaccccccg ttcagcccga ccgctgcgcc |
| | | ttatccggta actatcgtct tgagtccaac ccggtaagac acgacttatc |
| | | gccactggca gcagccactg gtaacaggat tagcagagcg aggtatgtag |
| | | gcggtgctac agagttcttg aagtggtggc ctaactacgg ctacactaga |
| | | aggacagtat ttggtatctg cgctctgctg aagccagtta ccttcggaaa |
| | | aagagttggt agctcttgat ccggcaaaca aaccaccgct ggtagcggtg |
| | | gtttttttgt ttgcaagcag cagattacgc gcagaaaaaa aggatctcaa |
| | | gaagatcctt tgatcttttc tacggggtct gacgctcagt ggaacgaaaa |
| | | ctcacgttaa gggattttgg TCATGAgttg tgtctcaaaa tctctgatgt |
| | | tacattgcac aagataaaaa tatatcatca tgaacaataa aactgtctgc |
| | | ttacataaac agtaatacaa ggggtgttat gagccatatt caacgggaaa |
| | | cgtcttgctc gaggccgcga ttaaattcca acatggatgc tgatttatat |
| | | gggtataaat gggctcgcga taatgtcggg caatcaggtg cgacaatcta |
| | | tcgattgtat gggaagcccg atgcgccaga gttgtttctg aaacatggca |
| | | aaggtagcgt tgccaatgat gttacagatg agatggtcag actaaactgg |
| | | ctgacggaat ttatgcctct tccgaccatc aagcatttta tccgtactcc |
| | | tgatgatgca tggttactca ccactgcgat ccccgggaaa acagcattcc |
| | | aggtattaga agaatatcct gattcaggtg aaaatattgt tgatgcgctg |
| | | gcagtgttcc tgcgccggtt gcattcgatt cctgtttgta attgtccttt |
| | | taacagcgat cgcgtatttc gtctcgctca ggcgcaatca cgaatgaata |
| | | acggtttggt tgatgcgagt gattttgatg acgagcgtaa tggctggcct |
| | | gttgaacaag tctggaaaga aatgcataag cttttgccat tctcaccgga |
| | | ttcagtcgtc actcatggtg atttctcact tgataacctt atttttgacg |
| | | aggggaaatt aataggttgt attgatgttg gacgagtcgg aatcgcagac |
| | | cgataccagg atcttgccat cctatggaac tgcctcggtg agttttctcc |
| | | ttcattacag aaacggcttt ttcaaaaata tggtattgat aatcctgata |
| | | tgaataaatt gcagtttcat ttgatgctcg atgagttttt ctaatcagaa |
| | | ttggttaatt ggttgtaaca ctggcagagc attacgctga cttgacggga |
| | | cggcggcttt gttgaataaa tcgaactttt gctgagttga aggatcCTCG |
| | | GGagttgtca gcctgtcccg cttataagat catacgccgt tatacGTTGT |
| | | TTACGCTTTG AGGAATTAAC C |

1. A composition comprising an orthogonal leucyl-tRNA (leucyl-O-tRNA), wherein the leucyl O-tRNA comprises an anticodon loop comprising a CU(X)ₙ XXXAA sequence, and comprises at least about a 25% suppression activity in presence of a cognate synthetase in response to a selector codon as compared to a control lacking the selector codon.

2. The composition of claim 1, wherein the leucyl-O-tRNA comprises a stem region comprising matched base pairs and a conserved discriminator base at position 73 and wherein the selector codon is amber codon.

3. The composition of claim 2, wherein the CU(X)ₙ XXXAA sequence comprises CUCUAAA sequence and n=0.

4. The composition of claim 2, wherein the leucyl-O-tRNA comprises a C:G base pair at position 3:70.

5. The composition of claim 1, wherein the leucyl-O-tRNA comprises:
a first pair selected from the group consisting of: U28:A42, G28:C42 and C28:G42; and,
a second pair selected from the group consisting of: G:49:C65 or C49:G65; and,
wherein the selector codon is a four-base codon.

6. The composition of claim 5, wherein the CU(X)ₙ XXXAA sequence comprises a CUUCCUAA sequence and n=1.

7. The composition of claim 5, wherein the first pair is C28:G42 and the second pair is C49:G65.

8. The composition of claim 1, wherein the CU(X)ₙ XXXAA sequence comprises a CUUCAAA sequence and n=0, and wherein the selector codon is an opal codon.

9. The composition of claim 1, wherein the leucyl-O-tRNA comprises or is encoded by a polynucleotide sequence as set forth in any one of SEQ ID NO.: 3, 6, 7 or 12, or a complementary polynucleotide sequence thereof.

10. The composition of claim 1, wherein the leucyl-O-tRNA and cognate synthetase, or a conservative variant thereof, are at least 50% as effective at suppressing a selector codon as a leucyl O-tRNA of SEQ ID NO: 3, 6, 7 or 12, in combination with a cognate synthetase.

11. The composition of claim 1, further comprising an orthogonal leucyl aminoacyl-tRNA synthetase (leucyl O-RS), wherein the leucyl O-RS preferentially aminoacylates the leucyl-O-tRNA with a selected amino acid.

12. The composition of claim 11, wherein the leucyl O-RS, or a portion thereof, is encoded by a polynucleotide sequence as set forth in any one of SEQ ID NO.: 13 or 14, or a complementary polynucleotide sequence thereof.

13. The composition of claim 11, wherein the leucyl O-RS comprises an amino acid sequence as set forth in any one of SEQ ID NO.: 15 or 16, or a conservative variation thereof.

14. The composition of claim 1, wherein the leucyl-O-tRNA is derived from an archael tRNA.

15. The composition of claim 1, wherein the leucyl-O-tRNA is derived from Halobacterium sp NRC-1.

16. The composition of claim 1, further comprising a translation system.

17. A cell comprising a translation system, wherein the translation system comprises:
an orthogonal leucyl-tRNA (leucyl-O-tRNA), wherein the leucyl-O-tRNA comprises at least about a 25% suppression activity in presence of a cognate synthetase in response to a selector codon as compared to a control lacking the selector codon;
an orthogonal aminoacyl-leucyl-tRNA synthetase (leucyl-O-RS); and,
a first selected amino acid;
wherein the leucyl O-tRNA comprises an anticodon loop comprising a CU(X)ₙ XXXAA sequence and recognizes the first selector codon, and the leucyl O-RS preferentially aminoacylates the leucyl O-tRNA with the first selected amino acid.

18. The cell of claim 17, wherein the leucyl-O-tRNA comprises or is encoded by a polynucleotide sequence as set forth in any one of SEQ ID NO.: 3, 6, 7 or 12, or a complementary polynucleotide sequence thereof, and wherein the leucyl O-RS comprises an amino acid sequence as set forth in any one of SEQ ID NO.: 15 or 16, or a conservative variation thereof.

19. The cell of claim 17, wherein the leucyl-O-tRNA and cognate synthetase, or a conservative variant thereof, are at least 50% as effective at suppressing a selector codon as a leucyl O-tRNA of SEQ ID NO: 3, 6, 7 or 12, in combination with a cognate synthetase.

20. The cell of claim 17, wherein the cell further comprises an additional different O-tRNA/O-RS pair and a second selected amino acid, wherein the O-tRNA recognizes a second selector codon and the O-RS preferentially aminoacylates the O-tRNA with the second selected amino acid.

21. The cell of claim 17, wherein the leucyl O-tRNA is derived from Halobacterium sp NRC-1 and the leucyl O-RS is derived from Methanobacterium thermoaautotropicum.

22. The cell of claim 17, wherein the cell is a eukaryotic cell.

23. The cell of claim 17, wherein the cell is a non-eukaryotic cell.

24. The cell of claim 23, wherein the non-eukaryotic cell is an E. coli cell.

25. The cell of claim 17, further comprising a nucleic acid that comprises a polynucleotide that encodes a polypeptide of interest, wherein the polynucleotide comprises or encodes a selector codon that is recognized by the leucyl O-tRNA.

26. An E. coli cell comprising:
an orthogonal leucyl-tRNA (leucyl-O-tRNA), wherein the leucyl-O-tRNA comprises at least about a 25% suppression activity in presence of a cognate synthetase in response to a selector codon as compared to a control lacking the selector codon;
an orthogonal leucyl aminoacyl-tRNA synthetase (leucyl-O-RS), wherein the leucyl O-RS preferentially aminoacylates the leucyl O-tRNA with a selected amino acid;
the selected amino acid; and,
a nucleic acid that comprises a polynucleotide that encodes a polypeptide of interest, wherein the polynucleotide comprises a selector codon that is recognized by the leucyl O-tRNA, and wherein the leucyl O-tRNA is derived from Halobacterium sp NRC-1 and the leucyl O-RS is derived from Methanobacterium thermoaautotropicum.

27-61. (canceled)


SEQUENCE LISTING (72 sequences)

SEQ ID NO: 1 - DNA, 88 bp, artificial
  mutant tRNA
gcgagggtagccaagctcggccaacggcgacggactctagatccgttctcgtaggagttcgagggttcgaatcccttccctcgcacca

SEQ ID NO: 2 - DNA, 88 bp, artificial
  mutant tRNA
gcgagggtagccaagctcggccaacggcgacggactctaaatccgttctcgtaggagttcgagggttcgaatcccttccctcgcacca

SEQ ID NO: 3 - DNA, 88 bp, artificial
  mutant tRNA
cccagggtagccaagctcggccaacggcgacggactctaaatccgttctcgtaggagttcgagggttcgaatcccttccctgggacca

SEQ ID NO: 4 - DNA, 89 bp, artificial
  mutant tRNA
gcgagggtagccaagctcggccaacggcgacggacttcctaatccgttctcgtaggagttcgagggttcgaatcccttccctcgcacca

SEQ ID NO: 5 - DNA, 89 bp, artificial
  mutant tRNA
gcgagggtagccaagctcggccaacggcgacggacttcctaatccgttctcgtaggagttcgagggttcgaatccctcccctcgcacca

SEQ ID NO: 6 - DNA, 89 bp, artificial
  mutant tRNA
gcgggggttgccgagcctggccaaaggcgccggacttcctaatccggtcccgtaggggttccggggttcaaatccccgcccccgcacca

SEQ ID NO: 7 - DNA, 88 bp, artificial
  mutant tRNA
gcgggggttgccgagcctggccaaaggcgccggacttcaaatccggtcccgtaggggttccggggttcaaatccccgcccccgcacca

SEQ ID NO: 8 - DNA, 77 bp, artificial
  mutant tRNA
ccggcggtagttcagcagggcagaacggcggactctaaatccgcatggcgctggttcaaatccggcccgccggacca

SEQ ID NO: 9 - DNA, 90 bp, Escherichia coli
ggagagatgccggagcggctgaacggaccggtctctaaaaccggagtaggggcaactctaccgggggttcaaatccccctctctccgcca

SEQ ID NO: 10 - DNA, 91 bp, artificial
  mutant tRNA
ggagagatgccggagcggctgaacggaccggtcttcctaaaccggagtaggggcaactctaccgggggttcaaatccccctctctccgcca

SEQ ID NO: 11 - RNA, 88 nt, Halobacterium sp. NRC-1
gcgaggguagccaagcucggccaacggcgacggacucaagauccguucucguaggaguucgaggguucgaaucccuucccucgcacca

SEQ ID NO: 12 - RNA, 89 nt, artificial
  consensus tRNA
gcggggguugccgagccuggccaaaggcgccggacuuccuaaucccgucccguagggguucggggguucaaauccccgcccccgcacca

SEQ ID NO: 13 - DNA, 2799 bp, Archaeoglobus fulgidus
atgagcgatttcaggataattgaggagaagtggcagaaggcgtgggagaaggacagaatttttgagtccgatcctaatgagaaggagaagttttttctcacaattccctatccttaccttaatggaaatcttcacgcaggtcacacgagaaccttcacaattggcgatgccttcgccagatacatgagaatgaagggctacaacgttctctttcccctcggctttcatgttacgggcaccccaatcattggccttgcggagctcatagccaagagggacgagaggacgatagaggtttacaccaaataccatgacgttccgctggaggacttgcttcagctcacaactccagagaaaatcgttgagtacttctcaagggaggcgctgcaggctttgaagagcataggctactccattgactggaggagggttttcaccacaaccgatgaagagtatcagagattcatcgagtggcagtactggaagctcaaggagcttggcctgattgtgaagggcacccaccccgtcagatactgcccccacgaccagaatcctgttgaagaccacgaccttctcgctggggaggaggcaactattgttgaatttaccgttataaagttcaggcttgaagatggagacctcattttcccctgtgcaactctccgtcccgaaaccgtgtttggcgtcacgaacatctgggtaaagccgacaacctacgtaattgccgaggtggatggggaaaagtggtttgtgagcaaagaggcttacgagaagctcacctacacggagaaaaaagtcaggctgctggaggaggttgatgcgtcgcagttcttcggcaagtacgtcatagtcccgctggtaaacagaaaagtgccaattctgcctgcagagtttgttgacaccgacaacgcaacaggagttgtgatgagcgttcccgcacacgctccttttgacctggctgccattgaggacttgaagagagacgaggaaacgctggcgaagtacggaattgacaaaagcgttgtagagagcataaagccaatagttctgattaagacggacattgaaggtgttcctgctgagaagctaataagagagcttggagtgaagagccagaaggacaaggagctgctggataaggcaaccaagaccctctacaagaaggagtaccacacgggaatcatgctggacaacacgatgaactatgctggaatgaaagtttctgaggcgaaggagagagttcatgaggatttggttaagcttggcttgggggatgttttctacgagttcagcgagaagcccgtaatctgcaggtgcggaacgaagtgcgttgttaaggttgttagggaccagtggttcctgaactactccaacagagagtggaaggagaaggttctgaatcaccttgaaaagatgcgaatcatccccgactactacaaggaggagttcaggaacaagattgagtggctcagggacaaggcttgtgccagaaggaaggggcttggaacgagaattccgtgggataaggagtggctcatcgagagcctttcagactcaacaatctacatggcctactacatccttgccaagtacatcaacgcaggattgctcaaggccgagaacatgactcccgagttcctcgactacgtgctgctgggcaaaggtgaggttgggaaagttgcggaagcttcaaaactcagcgtggagttaatccagcagatcagggacgacttcgagtactggtatcccgttgacctaagaagcagtggcaaggacttggttgcaaaccacctgctcttctacctcttccaccacgtcgccattttcccgccagataagtggccgagggcaattgccgtaaacggatacgtcagccttgagggcaagaagatgagcaagagcaaagggcccttgctaacgatgaagagggcggtgcagcagtatggtgcggatgtgacgaggctctacatcctccacgctgcagagtacgacagcgatgcggactggaagagcagagaggttgaagggcttgcaaaccacctcaggaggttctacaacctcgtgaaggagaactacctgaaagaggtgggagagctaacaaccctcgaccgctggcttgtgagcaggatgcagagggcaataaaggaagtgagggaggctatggacaacctgcagacgaggagggccgtgaatgccgccttcttcgagctcatgaacgacgtgagatggtatctgaggagaggaggtgagaacctcgctataatactggacgactggatcaagctcctcgccccctttgctccgcacatttgcgaggagctgtggcacttgaagcatgacagctacgtcagcctcgaaagctacccagaatacgacgaaaccagggttgacgaggaggcggagagaattgaggaatacctccgaaaccttgttgaggacattcaggaaatcaagaagtttgttagcgatgcgaaggaggtttacattgctcccgccgaagactggaaggttaaggcagcaaaggtcgttgctgaaagcggggatgttggggaggcgatgaagcagcttatgcaggacgaggagcttaggaagctcggcaaagaagtgtcaaatttcgtcaagaagattttcaaagacagaaagaagctgatgctagttaaggagtgggaagttctgcagcagaacctgaaatttattgagaatgagaccggactgaaggttattcttgatactcagagagttcctgaggagaagaggaggcaggcagttccgggcaagcccgcgatttatgttgcttaa

SEQ ID NO: 14 - DNA, 2814 bp, Methanobacterium thermoautotrophicum
gtggatattgaaagaaaatggcgtgatagatggagagatgctggcatatttcaggctgaccctgatgacagagaaaagatattcctcacagtcgcttacccctaccccagtggtgcgatgcacataggacacgggaggacctacactgtccctgatgtctatgcacggttcaagaggatgcagggctacaacgtcctgtttcccatggcctggcatgtcacaggggcccctgtcatagggatagcgcggaggattcagaggaaggatccctggaccctcaaaatctacagggaggtccacagggtccccgaggatgagcttgaacgtttcagtgaccctgagtacatagttgaatacttcagcagggaataccggtctgttatggaggatatgggctactccatcgactggaggcgtgaattcaaaaccacggatcccacctacagcaggttcatacagtggcagataaggaagctgagggaccttggcctcgtaaggaagggcgcccatcctgttaagtactgccctgaatgtgaaaaccctgtgggtgaccatgacctccttgagggtgagggggttgccataaaccagctcacactcctcaaattcaaacttggagactcatacctggtcgcagccaccttcaggcccgagacaatctatggggccaccaacctctggctgaaccctgatgaggattatgtgagggttgaaacaggtggtgaggagtggataataagcagggctgccgtggataatctttcacaccagaaactggacctcaaggtttccggtgacgtcaaccccggggacctgatagggatgtgcgtggagaatcctgtgacgggccaggaacaccccatactcccggcttccttcgttgaccctgaatatgccacaggtgttgtgttctctgtccctgcacatgcccctgcagacttcatagcccttgaggacctcaggacagaccatgaactccttgaaaggtacggtcttgaggatgtggttgctgatattgagcccgtgaatgtcatagcagtggatggctacggtgagttcccggcggccgaggttatagagaaatttggtgtcagaaaccaggaggacccccgccttgaggatgccaccggggagctatacaagatcgagcatgcgaggggtgttatgagcagccacatccctgtctatggtggtatgaaggtctctgaggcccgtgaggtcatcgctgatgaactgaaggaccagggccttgcagatgagatgtatgaattcgctgagcgacctgttatatgccgctgcggtggcaggtgcgttgtgagggtcatggaggaccagtggttcatgaagtactctgatgacgcctggaaggacctcgcccacaggtgcctcgatggcatgaagataatacccgaggaggtccgggccaactttgaatactacatcgactggctcaatgactgggcatgttcaaggaggataggccttggaacaaggctgccctgggatgagaggtggatcatcgaacccctcacagactcaacaatctacatggcatattacaccatcgcacaccgcctcagggagatggatgccggggagatggacgatgagttctttgatgccatattcctagatgattcaggaacctttgaggatctcagggaggaattccggtactggtacccccttgactggaggctctctgcaaaggacctcataggcaatcacctgacattccatatattccaccactcagccatattccctgagtcagggtggccccggggggctgtggtctttggtatgggccttcttgagggcaacaagatgtcatcctccaagggcaacgtcatactcctgagggatgccatcgagaagcacggtgcagacgtggtgcggctcttcctcatgtcctcagcagagccatggcaggactttgactggagggagagtgaggtcatcgggacccgcaggaggattgaatggttcagggaattcggagagagggtctcaggtatcctggatggtaggccagtcctcagtgaggttactccagctgaacctgaaagcttcattggaaggtggatgatgggtcagctgaaccagaggatacgtgaagccacaagggcccttgaatcattccagacaagaaaggcagttcaggaggcactctatctccttaaaaaggatgttgaccactaccttaagcgtgttgagggtagagttgatgatgaggttaaatctgtccttgcaaacgttctgcacgcctggataaggctcatggctccattcataccctacactgctgaggagatgtgggagaggtatggtggtgagggttttgtagcagaagctccatggcctgacttctcagatgatgcagagagcagggatgtgcaggttgcagaggagatggtccagaataccgttagagacattcaggaaatcatgaagatccttggatccaccccggagagggtccacatatacacctcaccaaaatggaaatgggatgtgctaagggtcgcagcagaggtaggaaaactagatatgggctccataatgggaagggtttcagctgagggcatccatgataacatgaaggaggttgctgaatttgtaaggaggatcatcagggaccttggtaaatcagaggttacggtgatagacgagtacagcgtactcatggatgcatctgattacattgaatcagaggttggagccagggttgtgatacacagcaaaccagactatgaccctgaaaacaaggctgtgaatgccgttcccctgaagccagccatataccttgaatga

SEQ ID NO: 15 - protein, 932 aa, Archaeoglobus fulgidus
MSDFRIIEEKWQKAWEKDRIFESDPNEKEKFFLTIPYPYLNGNLHAGHTRTFTIGDAFARYMRMKGYNVLFPLGFHVTGTPIIGLAELIAKRDERTIEVYTKYHDVPLEDLLQLTTPEKIVEYFSREALQALKSIGYSIDWRRVFTTTDEEYQRFIEWQYWKLKELGLIVKGTHPVRYCPHDQNPVEDHDLLAGEEATIVEFTVIKFRLEDGDLIFPCATLRPETVFGVTNIWVKPTTYVIAEVDGEKWFVSKEAYEKLTYTEKKVRLLEEVDASQFFGKYVIVPLVNRKVPILPAEFVDTDNATGVVMSVPAHAPFDLAAIEDLKRDEETLAKYGIDKSVVESIKPIVLIKTDIEGVPAEKLIRELGVKSQKDKELLDKATKTLYKKEYHTGIMLDNTMNYAGMKVSEAKERVHEDLVKLGLGDVFYEFSEKPVICRCGTKCVVKVVRDQWFLNYSNREWKEKVLNHLEKMRIIPDYYKEEFRNKIEWLRDKACARRKGLGTRIPWDKEWLIESLSDSTIYMAYYILAKYINAGLLKAENMTPEFLDYVLLGKGEVGKVAEASKLSVELIQQIRDDFEYWYPVDLRSSGKDLVANHLLFYLFHHVAIFPPDKWPRAIAVNGYVSLEGKKMSKSKGPLLTMKRAVQQYGADVTRLYILHAAEYDSDADWKSREVEGLANHLRRFYNLVKENYLKEVGELTTLDRWLVSRMQRAIKEVREAMDNLQTRRAVNAAFFELMNDVRWYLRRGGENLAIILDDWIKLLAPFAPHICEELWHLKHDSYVSLESYPEYDETRVDEEAERIEEYLRNLVEDIQEIKKFVSDAKEVYIAPAEDWKVKAAKVVAESGDVGEAMKQLMQDEELRKLGKEVSNFVKKIFKDRKKLMLVKEWEVLQQNLKFIENETGLKVILDTQRVPEEKRRQAVPGKPAIYVA

SEQ ID NO: 16 - protein, 937 aa, Methanobacterium thermoautotrophicum
VDIERKWRDRWRDAGIFQADPDDREKIFLTVAYPYPSGAMHIGHGRTYTVPDVYARFKRMQGYNVLFPMAWHVTGAPVIGIARRIQRKDPWTLKIYREVHRVPEDELERFSDPEYIVEYFSREYRSVMEDMGYSIDWRREFKTTDPTYSRFIQWQIRKLRDLGLVRKGAHPVKYCPECENPVGDHDLLEGEGVAINQLTLLKFKLGDSYLVAATFRPETIYGATNLWLNPDEDYVRVETGGEEWIISRAAVDNLSHQKLDLKVSGDVNPGDLIGMCVENPVTGQEHPILPASFVDPEYATGVVFSVPAHAPADFIALEDLRTDHELLERYGLEDVVADIEPVNVIAVDGYGEFPAAEVIEKFGVRNQEDPRLEDATGELYKIEHARGVMSSHIPVYGGMKVSEAREVIADELKDQGLADEMYEFAERPVICRCGGRCVVRVMEDQWFMKYSDDAWKDLAHRCLDGMKIIPEEVRANFEYYIDWLNDWACSRRIGLGTRLPWDERWIIEPLTDSTIYMAYYTIAHRLREMDAGEMDDEFFDAIFLDDSGTFEDLREEFRYWYPLDWRLSAKDLIGNHLTFHIFHHSAIFPESGWPRGAVVFGMGLLEGNKMSSSKGNVILLRDAIEKHGADVVRLFLMSSAEPWQDFDWRESEVIGTRRRIEWFREFGERVSGILDGRPVLSEVTPAEPESFIGRWMMGQLNQRIREATRALESFQTRKAVQEALYLLKKDVDHYLKRVEGRVDDEVKSVLANVLHAWIRLMAPFIPYTAEEMWERYGGEGFVAEAPWPDFSDDAESRDVQVAEEMVQNTVRDIQEIMKILGSTPERVHIYTSPKWKWDVLRVAAEVGKLDMGSIMGRVSAEGIHDNMKEVAEFVRRIIRDLGKSEVTVIDEYSVLMDASDYIESEVGARVVIHSKPDYDPENKAVNAVPLKPAIYLE

SEQ ID NO: 17 - DNA, 4814 bp, artificial
  mutant pACKO-Bla
gaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctaggagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggcaccaccaccaccaccactaacccgggaccaagtttactcatatatactttagattgatttaaaacttcatttttaatttaaaaggatctaggtgaagatcctttttgataatctcatgaccaaaatcccttaacggcatgcaccattccttgcggcggcggtgctcaacggcctcaacctactactgggctgcttcctaatgcaggagtcgcataagggagagcgtctggcgaaagggggatgtgctgcaaggcgattaagttgggtaacgccagggttttcccagtcacgacgttgtaaaacgacggccagtgccaagcttaaaaaaaatccttagctttcgctaaggatctgcagttataatctctttctaattggctctaaaatctttataagttcttcagctacagcattttttaaatccattggatgcaattccttatttttaaataaactctctaactcctcatagctattaactgtcaaatctccaccaaatttttctggcctttttatggttaaaggatattcaaggaagtatttagctatctccattattggatttccttcaacaactccagctgggcagtatgctttctttatcttagccctaatctcttctggagagtcatcaacagctataaaattcccttttgaagaactcatctttccttctccatccaaacccgttaagacagggttgtgaatacaaacaaccttttttggtaaaagctcccttgctaacatgtgtatttttctctgctccatccctccaactgcaacatcaacgcctaaataatgaatatcattaacctgcattattggatagataacttcagcaacctttggattttcatcctctcttgctataagttccatactccttcttgctctttttaaggtagtttttaaagccaatctatagacattcagtgtataatccttatcaagctggaattcagcgttacaagtattacacaaagttttttatgttgagaatatttttttgatggggcgccacttatttttgatcgttcgctcaaagaagcggcgccagggntgtttttcttttcaccagtnagacgggcaacagaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 18 - DNA, 4813 bp, artificial
  mutant pACKO-Bla
gaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccttaggcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggcaccaccaccaccaccactaacccgggaccaagtttactcatatatactttagattgatttaaaacttcatttttaatttaaaaggatctaggtgaagatcctttttgataatctcatgaccaaaatcccttaacggcatgcaccattccttgcggcggcggtgctcaacggcctcaacctactactgggctgcttcctaatgcaggagtcgcataagggagagcgtctggcgaaagggggatgtgctgcaaggcgattaagttgggtaacgccagggttttcccagtcacgacgttgtaaaacgacggccagtgccaagcttaaaaaaaatccttagctttcgctaaggatctgcagttataatctctttctaattggctctaaaatctttataagttcttcagctacagcattttttaaatccattggatgcaattccttatttttaaataaactctctaactcctcatagctattaactgtcaaatctccaccaaatttttctggcctttttatggttaaaggatattcaaggaagtatttagctatctccattattggatttccttcaacaactccagctgggcagtatgctttctttatcttagccctaatctcttctggagagtcatcaacagctataaaattcccttttgaagaactcatctttccttctccatccaaacccgttaagacagggttgtgaatacaaacaaccttttttggtaaaagctcccttgctaacatgtgtatttttctctgctccatccctccaactgcaacatcaacgcctaaataatgaatatcattaacctgcattattggatagataacttcagcaacctttggattttcatcctctcttgctataagttccatactccttcttgctctttttaaggtagtttttaaagccaatctatagacattcagtgtataatccttatcaagctggaattcagcgttacaagtattacacaaagttttttatgttgagaatatttttttgatggggcgccacttatttttgatcgttcgctcaaagaagcggcgccagggntgtttttcttttcaccagtnagacgggcaacagaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 19 - DNA, 4813 bp, artificial
  mutant pACKO-Bla
gaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccttgagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggcaccaccaccaccaccactaacccgggaccaagtttactcatatatactttagattgatttaaaacttcatttttaatttaaaaggatctaggtgaagatcctttttgataatctcatgaccaaaatcccttaacggcatgcaccattccttgcggcggcggtgctcaacggcctcaacctactactgggctgcttcctaatgcaggagtcgcataagggagagcgtctggcgaaagggggatgtgctgcaaggcgattaagttgggtaacgccagggttttcccagtcacgacgttgtaaaacgacggccagtgccaagcttaaaaaaaatccttagctttcgctaaggatctgcagttataatctctttctaattggctctaaaatctttataagttcttcagctacagcattttttaaatccattggatgcaattccttatttttaaataaactctctaactcctcatagctattaactgtcaaatctccaccaaatttttctggcctttttatggttaaaggatattcaaggaagtatttagctatctccattattggatttccttcaacaactccagctgggcagtatgctttctttatcttagccctaatctcttctggagagtcatcaacagctataaaattcccttttgaagaactcatctttccttctccatccaaacccgttaagacagggttgtgaatacaaacaaccttttttggtaaaagctcccttgctaacatgtgtatttttctctgctccatccctccaactgcaacatcaacgcctaaataatgaatatcattaacctgcattattggatagataacttcagcaacctttggattttcatcctctcttgctataagttccatactccttcttgctctttttaaggtagtttttaaagccaatctatagacattcagtgtataatccttatcaagctggaattcagcgttacaagtattacacaaagttttttatgttgagaatatttttttgatggggcgccacttatttttgatcgttcgctcaaagaagcggcgccagggntgtttttcttttcaccagtnagacgggcaacagaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 20 - DNA, 4338 bp, artificial
  plasmid pACKO-Bla
gaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacactagtgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaacccgggaccaagtttactcatatatactttagattgatttaaaacttcatttttaatttaaaaggatctaggtgaagatcctttttgataatctcatgaccaaaatcccttaacggcatgcaccattccttgcggcggcggtgctcaacggcctcaacctactactgggctgcttcctaatgcaggagtcgcataagggagagcgtctggcgaaagggggatgtgctgcaaggcgattaagttgggtaacgccagggttttcccagtcacgacgttgtaaaacgacggccagtgccaagcttaaaaaaaatccttagctttcgctaaggatctgcagttataatctctttctaattggctctaaaatctttataagttcttcagctacagcattttttaaatccattggatgcaattccttatttttaaataaactctctaactcctcatagctattaactgtcaaatctccaccaaatttttctggcctttttatggttaaaggatattcaaggaagtatttagctatctccattattggatttccttcaacaactccagctgggcagtatgctttctttatcttagccctaatctcttctggagagtcatcaacagctataaaattcccttttgaagaactcatctttccttctccatccaaacccgttaagacagggttgtgaatacaaacaaccttttttggtaaaagctcccttgctaacatgtgtatttttctctgctccatccctccaactgcaacatcaacgcctaaataatgaatatcattaacctgcattattggatagataacttcagcaacctttggattttcatcctctcttgctataagttccatactccttcttgctctttttaaggtagtttttaaagccaatctatagacattcagtgtataatccttatcaagctggaattcagcgttacaagtattacacaaagttttttatgttgagaatatttttttgatggggcgccacttatttttgatcgttcgctcaaagaagcggcgccagggntgtttttcttttcaccagtnagacgggcaacagaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 21 - DNA, 2271 bp, artificial
  plasmid pKQ
atggatccgagctcgagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgatatctgcttttcttcgcgaattaattccgcttcgcaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcctcgggagttgtcagcctgtcccgcttataagatcatacgccgttatacgttgtttacgctttgaggaattaacc

SEQ ID NO: 22 - protein, 10 aa, Escherichia coli
VVLQRRDWEN

SEQ ID NO: 23 - DNA, 28 bp, artificial
  oligonucleotide primer
ggtttccatgggagagcaagccacctac

SEQ ID NO: 24 - DNA, 29 bp, artificial
  oligonucleotide primer
ggtttggaattcagtcgtcggcttcgtcg

SEQ ID NO: 25 - DNA, 32 bp, artificial
  oligonucleotide primer
cgaaaccatggaagagcaataccgcccggaag

SEQ ID NO: 26 - DNA, 34 bp, artificial
  oligonucleotide primer
ccaaagaattcccgccaacgaccagattgaggag

SEQ ID NO: 27 - DNA, 29 bp, artificial
  oligonucleotide primer
cgaaaccatggttatgattgactttaaag

SEQ ID NO: 28 - DNA, 34 bp, artificial
  oligonucleotide primer
cgaaaggtaccttgtattcaagataaatagctgg

SEQ ID NO: 29 - DNA, 31 bp, artificial
  oligonucleotide primer
gcgaaccatgggcgatttcaggataattgag

SEQ ID NO: 30 - DNA, 30 bp, artificial
  oligonucleotide primer
caattggtaccttaagcaacataaatcgcg

SEQ ID NO: 31 - DNA, 35 bp, artificial
  oligonucleotide primer
ggattatcatgaagcgactaaaggccgtggaggag

SEQ ID NO: 32 - DNA, 32 bp, artificial
  oligonucleotide primer
cacttgaattcttagcctcctctcttctccgc

SEQ ID NO: 33 - DNA, 29 bp, artificial
  oligonucleotide primer
cgaatccatggctgagcttaacttcaagg

SEQ ID NO: 34 - DNA, 29 bp, artificial
  oligonucleotide primer
ggatggatatcactcgatgaagatggcag

SEQ ID NO: 35 - DNA, 36 bp, artificial
  oligonucleotide primer
ggagacgtctctcatggatattgaaagaaaatggcg

SEQ ID NO: 36 - DNA, 35 bp, artificial
  oligonucleotide primer
cgttacgtctcgaattggaaaagagctgtctgagg

SEQ ID NO: 37 - RNA, 88 nt, Pyrococcus furiosus
gcgggggugcccgagccuggccaaaggggccggacuuaagauccggugccguagggcugcgcggguucgaaucccgccccccgcacca

SEQ ID NO: 38 - RNA, 88 nt, Pyrococcus horikoshi; identical to SEQ ID NO: 37

SEQ ID NO: 39 - RNA, 87 nt, Pyrococcus abyssi
gcgggggugcccgagccuggccaaaggggccggacuuaagauccggugccguaggcugcgcggguucgaaucccgccccccgcacca

SEQ ID NO: 40 - RNA, 88 nt, Aeuropyrum pernix
gcgggggugcccgagccuggccaaaggggucgggcugaggacccgauggcguaggccugcguggguucaaaucccaccccccgcacca

SEQ ID NO: 41 - RNA, 88 nt, Aeuropyrum pernix
gcgggggugcccgagccuggccaaaggggucgggcucaggacccgauggcguaggccugcguggguucaaaucccaccccccgcacca

SEQ ID NO: 42 - RNA, 88 nt, Aeuropyrum pernix
gcgggggugcccgagccuggccaaaggggucgggcuuaggacccgauggcguaggccugcguggguucaaaucccaccccccgcacca

SEQ ID NO: 43 - RNA, 88 nt, Aeuropyrum pernix
gcgggggugcccgagccuggccaaagggggcgggcuuaagacccguuggcguaggccugcguggguucaaaucccaccccccgcacca

SEQ ID NO: 44 - RNA, 88 nt, Aeuropyrum pernix
gcgggggugcccgagccagggcgaagggggcgggcucaagacccguuggcguaggccggcguggguucaaaucccaccccccgcacca

SEQ ID NO: 45 - RNA, 87 nt, Methanobacterium thermoautotrophicum
gcgggggugcccgagcuggccaaaggggacaggcuuaggaccuguuggcguaggccuaccaggguucgaaucccugcccccgcacca

SEQ ID NO: 46 - RNA, 86 nt, Archaeoglobus fulgidus
gcggggguugccgagcggacaaaggcgcaggauugaggguccugucccguagggguucgaggguucgaaucccuccccccgcacca

SEQ ID NO: 47 - RNA, 86 nt, Archaeoglobus fulgidus
gcggggguugccgagcggacaaaggcgcgggauucagggucccgucccguagggguucgaggguucgaaucccuccccccgcacca

SEQ ID NO: 48 - RNA, 86 nt, Archaeoglobus fulgidus
gcggggguugccgagcggccaaaggcgcuggauuuaggguccagucccguagggguucgaggguucgaaucccuccccccgcacca

SEQ ID NO: 49 - RNA, 86 nt, Halobacterium sp. NRC-1
gcgaggguggccgagcggccaaaggcggcgggcuuaagacccgcucccguagggguucguggguucgaaucccaccucucgcacca

SEQ ID NO: 50 - RNA, 86 nt, Methanobacterium thermoautotrophicum
gcaggggugcccgagcggccaaagggggaggacuuaagauccucuggcgcaggccuucgaggguucgaaucccuuccccugcacca

SEQ ID NO: 51 - RNA, 88 nt, Methanococcus jannaschii
gcaggggucgccaagccuggccaaaggcgcugggccuaggacccagucccguagggguuccaggguucaaaucccugccccugcacca

SEQ ID NO: 52 - RNA, 88 nt, Methanococcus jannaschii
gcaggggucgccaagccuggccaaaggcgccggacuuaagauccggucccguagggguucggggguucaaauccccuccccugcacca

SEQ ID NO: 53 - RNA, 88 nt, Pyrococcus abyssi
gcggggguugccgagccuggucaaaggcgggggacucaagauccccucccguagggguuccgggguucaaauccccgcccccgcacca

SEQ ID NO: 54 - RNA, 88 nt, Pyrococcus furiosus; identical to SEQ ID NO: 53

SEQ ID NO: 55 - RNA, 88 nt, Pyrococcus horikoshi
gcggggguugccgagccuggucaaaggcgggggacucaagauccccucccguagggguuccgggguucgaauccccgcccccgcacca

SEQ ID NO: 56 - RNA, 88 nt, Pyrococcus abyssi
gcggggguugccgagccuggucaaaggcgcgggauugagggucccgucccguagggguuccgggguucaaauccccgcccccgcacca

SEQ ID NO: 57 - RNA, 88 nt, Pyrococcus furiosus; identical to SEQ ID NO: 56

SEQ ID NO: 58 - RNA, 88 nt, Pyrococcus horikoshi; identical to SEQ ID NO: 56

SEQ ID NO: 59 - RNA, 88 nt, Pyrococcus abyssi
gcggggguugccgagccuggucaaaggcgcgggauucagggucccgucccguagggguuccgggguucaaauccccgcccccgcacca

SEQ ID NO: 60 - RNA, 88 nt, Pyrococcus furiosus; identical to SEQ ID NO: 59

SEQ ID NO: 61 - RNA, 88 nt, Pyrococcus horikoshi; identical to SEQ ID NO: 59

SEQ ID NO: 62 - RNA, 88 nt, Pyrococcus furiosus
gcggggguugccgagccuggucaaaggcgcgggauuuagggucccgucccguagggguuccgggguucaaauccccgcccccgcacca

SEQ ID NO: 63 - RNA, 88 nt, Pyrococcus horikoshi; identical to SEQ ID NO: 62

SEQ ID NO: 64 - RNA, 88 nt, Pyrococcus abyssi
gcggggguugccgagccuggccaaaggcgcgggauuuagggucccgucccguagggguuccgggguucaaauccccgcccccgcacca

SEQ ID NO: 65 - RNA, 88 nt, Methanococcus jannaschii
gcgggggucgccaagccaggucaaaggcgccagauugagggucuggucccguagggguucgcggguucaaaucccgucccccgcacca

SEQ ID NO: 66 - RNA, 88 nt, Halobacterium sp. NRC-1; identical to SEQ ID NO: 11

SEQ ID NO: 67 - RNA, 87 nt, Archaeoglobus fulgidus
gcggggguugccgagccaggaaaaggcgcagggcuuaagacccugucccgaagggguccgcggguucgaaucccgccccccgcacca

SEQ ID NO: 68 - RNA, 87 nt, Halobacterium sp. NRC-1
gcgcggguagccaagccaggaaacggcguagcgcuuaggacgcuaucccguagggguccgccgguucaaauccggucccgcgcacca

SEQ ID NO: 69 - RNA, 88 nt, Halobacterium sp. NRC-1
gcguggguagccgagcuaggucaaaggcgcagcguugagggcgcuguccuguagagguucgccgguucgaauccggucccacgcacca

SEQ ID NO: 70 - RNA, 88 nt, Halobacterium sp. NRC-1
gcagggauagccaaguuuggccaaaggcgcagcguucagggcgcugucccguagggguucgcagguucaaauccugcucccugcacca

SEQ ID NO: 71 - RNA, 86 nt, Methanobacterium thermoautotrophicum
gcagggguggucgagcggucaaaggcgcuagguugagggccuagugggggagcccuucgcggguucgaaucccguccccugcacca

SEQ ID NO: 72 - RNA, 88 nt, Artificial
  consensus tRNA
gcggggguugccgagccuggccaaaggcgcnggacuuaggauccngucccguagggguucggggguucaaauccccncccccgcacca